(19) United States
(12) Patent Application Publication
(10) Pub. No.: US 2006/0240021 A1
(43) Pub. Date: Oct. 26, 2006

(54) Mammalian genes involved in rapamycin resistance and tumorgenesis: rapr7 genes

(76) Inventors: Limin Li, North Bethesda, MD (US); Bahman Aghdasi, Takoma Park, MD (US)

Correspondence Address: MERCHANT & GOULD PC, P.O. BOX 2903, MINNEAPOLIS, MN, 55402-0903, US

(21) Appl. No.: 10/524,426

(22) PCT Filed: Aug. 18, 2003
(86) PCT No.: PCT/US03/26073

§ 371 Date: Dec. 12, 2005

Related U.S. Application Data
(60) Provisional application No. 60/404,311, filed on Aug. 15, 2002.

(51) Int. Cl. A61K 39/395; C12Q 1/68; C07H 21/04; C12P 21/06; C07K 14/82; C07K 16/30

(52) U.S. Cl. 424/155.1; 435/6; 435/69.1; 435/320.1; 435/325; 530/350; 530/388.8; 536/23.5

(57) ABSTRACT

The invention provides nucleotide sequence of a novel mammalian gene which is involved in rapamycin resistance and tumorgenesis the RapR7 gene, and amino acid sequences of its encoded proteins, and fragments and derivatives and analogs thereof. The present invention also provides methods and compositions for regulating rapamycin resistance and/or tumorgenesis by modulating the expression and/or the activity of RapR7 gene. The invention also provides methods and compositions for treatment of diseases, e.g., cancers, by modulating the expression and/or activity of RapR7 gene. The invention also provides methods and compositions for diagnosing and screening RapR7 mediated rapamycin resistance and/or tumorgenesis in patients. The invention further provides host cells whose RapR7 gene can be reversibly overexpressed, and to methods of using the RapR7 gene in evaluation and screening for drugs which regulate rapamycin resistance and/or tumorgenesis.

This application claims the benefit under 35 U.S.C. § 119(e) of U.S. Provisional Patent Application No. 60/404,311, filed on Aug. 15, 2002, which is incorporated by reference herein in its entirety.

### 1. FIELD OF THE INVENTION

The invention relates to a novel mammalian gene, termed the RapR7 gene, which is involved in rapamycin resistance and tumorgenesis, and to its products and derivatives and analogs thereof. The invention relates to fragments (and derivatives and analogs thereof) of a mammalian RapR7 gene product. The invention also provides methods of production of mammalian RapR7 gene products, and derivatives and analogs thereof. The present invention also relates to methods and compositions for regulating rapamycin resistance and tumorgenesis by modulating the expression of and/or activity of a RapR7 gene. The compositions of the invention include but are not limited to nucleic acids encoding RapR7 gene products and homologues, analogues, and deletions thereof, as well as antisense, ribozyme, triple helix, and double-stranded RNA. Also provided are RapR7 antibody and polypeptide molecules and small organic or inorganic molecules that bind to a RapR7 protein. The invention also relates to methods and compositions for treatment of diseases, e.g., cancers, by modulating the expression and/or activity of RapR7 gene alone or in conjunction with a rapamycin therapy. The invention also relates to methods and compositions for diagnosing and screening RapR7-mediated rapamycin resistance and/or tumorgenesis in patients. The invention further relates to host cells whose RapR7 gene can be reversibly activated/enhanced, and to methods of using RapR7 gene in evaluation and screening for drugs which regulate rapamycin resistance and/or tumorgenesis. The invention also relates to methods for generating genetically modified cells having altered sensitivity to rapamycin by knocking out a gene which mediates rapamycin resistance.

### 2. BACKGROUND OF THE INVENTION

Rapamycin (also called sirolimus) is a lipophilic macrolide which was isolated in 1975 as a fungicide from a strain of Streptomyces hygroscopicus found in a soil sample on Easter Island (see, e.g., Huang et al., 2001, Cancer and Metastasis Rev. 20:69-78; Sehgal et al., 1994, Medicinal Research Review 14:1-22). Total synthesis of rapamycin has been reported (see, e.g., Nicolaou et al., 1993, J. Am. Chem. Soc. 115:4419; Hayward et al., 1993, J. Am. Chem. Soc. 115: 9345). Rapamycin, or 9,10,12,13,14,21,22,23,24,25,26,27,32,33,34,34a-hexadecahydro-9,27-dihydroxy-3-[2-(4-hydroxy-3-methoxycyclohexyl)-1-me thylethyl]-10,21-dimethoxy-6,8,12,14,20,26-hexamethyl-23,27-epoxy-3H-pyrido[2,1-c][1,4]oxaazacyclohentricontine-1,5,11,28,29(4H,6H,31H)-pentone, comprises a 31-membered ring including a pipecolinyl group and pyranose ring, a conjugated triene system and a tri-carbonyl region. It has 15 chiral centers, and thus a large number of possible stereoisomers. Rapamycin targets the protein mTOR (the mammalian target of rapamycin, a homolog of TOR1 and TOR2, targets of rapamycin 1 and 2 in yeast), a serine/threonine kinase belonging to the phosphatidylinositol 3-kinase (PI3K) family of kinases (see, e.g., Huang et al., 2001, Cancer and Metastasis Rev. 20:69-78; Yu et al., 2001, Endocrine-Related Cancer 8:249-258; and Sabers et al., 1995, J. Biol. Chem. 270:815-822). mTOR has been identified as a central integrator of extra- and intracellular signals that initiate translation and transcription required for cell growth and proliferation (see, e.g., Huang et al., 2001, Cancer and Metastasis Rev. 20:69-78). In its action, rapamycin first binds to an intracellular receptor called FKBP-12 (“FK506 Binding Protein 12”). The rapamycin-FKBP-12 complex inhibits mTOR and therefore one or more of its downstream pathways, e.g., 4E-BP1 and p70S6K, to cause G1 cell cycle arrest. Derivatives of rapamycin, e.g., cell cycle inhibitor-779 (CCI-779), which is a rapamycin ester, are also reported to have such effect (see, e.g., Huang et al., 2001, Cancer and Metastasis Rev. 20:69-78; Yu et al., 2001, Endocrine-Related Cancer 8:249-258).

Rapamycin has been approved by the FDA as an immunosuppressant for prevention and treatment of graft rejection in organ transplant recipients and is currently marketed under the trade name “Rapamune®” by Wyeth. As an immunosuppressant, Rapamycin demonstrates a different mechanism of action as compared to traditional immunosuppressants in that rapamycin blocks the immune response by inhibiting the function of mTOR, thereby causing programmed cell death, or apoptosis, in T cells. Other commonly used immunosuppressants, such as cyclosporin and FK-506, work differently by binding to calcineurin, thereby blocking the Ca²⁺-dependent signaling pathway to the nucleus of the T cell. These latter immunosuppressants may have severe side effects because they also inhibit calcineurin activity in non-immune cells. In contrast, rapamycin selectively blocks the proliferation of T cells.

Rapamycin is also under clinical trial as a cancer chemotherapy drug due to its ability to cause cell cycle arrest in the G1 phase and to induce apoptosis (see, e.g., Huang et al., 2001, Cancer and Metastasis Rev. 20:69-78; Yu et al., 2001, Endocrine-Related Cancer 8:249-258; Mills et al., 2001, Proc. Natl. Acad. Sci. USA 98:10031-10033; Neshat et al., 2001, Proc. Natl. Acad. Sci. USA 98:10314-10319; and Podsypanina et al., 2001, Proc. Natl. Acad. Sci. USA 98:10320-10325). Rapamycin has been shown to be able to arrest the growth of a variety of malignant cells, including cells derived from rhabdomyosarcoma, neuroblastoma and glioblastoma, small cell lung cancer, osteoscarcoma, pancreatic cancer, breast and prostate cancer, murine melanoma and leukemia, and B-cell lymphoma

However, many cell lines have been found to exhibit resistance to the growth-inhibitory effect of rapamycin (see, e.g., Huang et al., 2001, Cancer and Metastasis Rev. 20:69-78). Resistance to rapamycin has been reported as a result of mutations in TOR or mTOR. In yeast, strains which have in TOR1 and/or TOR2 mutations that render the encoded proteins lacking the ability for rapamycin-FKBP-12 complex binding have been shown to be resistant to rapamycin completely (Heitman et al., 1991, Science 253:905-909). In mammals, a mutant of mTOR which exhibits reduced affinity for rapamycin-FKBP-12 complex has been reported to cause a high level of resistance to rapamycin (Chen et al., 1995, Proc. Natl. Acad. Sci. USA 92:4947-4951). Resistance to rapamycin has also been reported as a result of mutations in FKBP-12. For example, a resistant phenotype has been shown to be associated with a mutation in a mammalian homolog of FKBP-12 which leads to decreased binding of rapamycin. Mutations in downstream cellular constituents, e.g., p70S6K, that may confer rapamycin resistance have also been reported (see, e.g., Huang et. al., 2001, Cancer and Metastasis Rev. 20:69-78).

In addition to stable rapamycin resistant phenotypes such as those resulting from genetic mutations, acquired rapamycin resistance in cell lines has also been reported (see, e.g., Huang et al., 2001, Cancer and Metastasis Rev. 20:69-78; Dilling et al., 2000, Proceedings of 91^{st} Annual Meeting of the AACR #5110). Such rapamycin resistant cell lines were obtained by growing the cells in continuous and increasing concentrations of rapamycin. The cell lines can be reverted to rapamycin sensitive by growing the cells in the absence of rapamycin. Furthermore, genetic mutations in certain tumor suppressor genes, e.g., p53 and PTEN (“phosphatase and tensin homolog deleted on chromosome ten”), have been reported to result in rapamycin hypersensitivity (see, e.g., Huang et al., 2001, Cancer and Metastasis Rev. 20:69-78; Huang et al., 2001, Cancer Research 61:3373-3381; Yu et al., 2001, Proceedings of 92^{nd} Annual Meeting of the AACR #5110).

Considering the central role of mTOR in cell signaling and rapamycin as a potent anti-cancer drug candidate, additional cellular constituents and/or pathways, both upstream and downstream of mTOR, that confer rapamycin resistance may exist. Such cellular constituents and/or pathways may also play a role in tumorgenesis. However, such upstream or downstream constituents of the pathways are not yet known.

Citation of references hereinabove shall not be construed as an admission that such references are prior art to the present invention.

### 3. SUMMARY OF THE INVENTION

The invention provides nucleotide sequences of a novel mammalian gene, the RapR7 gene, which is involved in rapamycin resistance and tumorgenesis, and amino acid sequences of the encoded proteins, and derivatives and analogs thereof. In one embodiment, the invention provides a purified mammalian RapR7 protein. In a preferred embodiment, the invention provides a RapR7 protein which comprises the amino acid sequence substantially as set forth in SEQ ID NO:4 or 7. In a preferred embodiment, the invention provides a RapR7 protein which comprises the amino acid sequence substantially as set forth in SEQ ID NO:3 or 6. In another preferred embodiment, the invention provides a RapR7 protein which is encoded by a nucleic acid capable of hybridizing to a DNA having a sequence consisting of the coding region of SEQ ID NO:2 or 5.

The invention provides an isolated nucleic acid encoding a mammalian RapR7 protein. In one embodiment, the invention provides a nucleic acid molecule comprising a nucleotide sequence as set forth in SEQ ID NO:2 or 5. In a preferred embodiment, the nucleic acid molecule of the invention is a DNA molecule. The invention also provides an isolated nucleic acid comprising a nucleotide sequence complementary to a nucleotide sequence encoding a mammalian RapR7 protein. The invention also provides an isolated nucleic acid comprising a nucleotide sequence that is hybridizable to a nucleotide sequence encoding a mammalian RapR7 protein.

The invention also provides derivatives and analogs of a protein encoded by a RapR7 gene. In one embodiment, the invention provides a purified derivative or analog of a protein which displays one or more functional activities of a mammalian RapR7 protein. In a preferred embodiment, the invention provides a RapR7 derivative or analog which is capable of binding to an antibody directed against a mammalian RapR7 protein.

The invention also provides fragments of a protein encoded by a RapR7 gene, or a derivative or analog thereof. In one embodiment, the invention provides a purified fragment of a mammalian RapR7 protein. In a preferred embodiment, the invention provides a RapR7 fragment which comprises a PHD domain of a mammalian RapR7 protein. In a specific embodiment, the invention provides a fragment of a human RapR7 protein which comprises amino acids 217-263 or 326-381 of the human RapR7 protein. In another preferred embodiment, the invention provides a RapR7 fragment which comprises a coiled-coil domain of a mammalian RapR7 protein. In a specific embodiment, the invention provides a fragment of a human RapR7 protein which comprises amino acids 163-190 of the human RapR7 protein. In another preferred embodiment, the invention provides a RapR7 fragment which comprises a second peroximal domain of a mammalian RapR7 protein. In a specific embodiment, the invention provides a fragment of a human RapR7 protein which comprises amino acids 514-522 of the human RapR7 protein. The invention also provides a molecule which comprises any of such fragment of a mammalian RapR7 protein.

The invention also provides a protein comprising an amino acid sequence that has at least 60% or at least 90% identity to a domain of a mammalian RapR7 protein, in which the percentage identity is determined over an amino acid sequence of identical size to the domain. The invention also provides a polypeptide comprising a fragment of a mammalian RapR7 protein consisting of at least 6 amino acids fused via a covalent bond to an amino acid sequence of a second peptide which is not comprised in a mammalian RapR7 protein. In a preferred embodiment of the invention, such fragment of the mammalian RapR7 protein is a fragment capable of binding to an anti-RapR7 protein antibody. In another preferred embodiment, the fragment that is capable of binding to an anti-RapR7 protein antibody lacks one or more domains of the RapR7 protein.

The invention also provides an antibody which is capable of binding to a mammalian RapR7 protein. In a preferred embodiment, the antibody of the invention is a monoclonal antibody. In another preferred embodiment, the invention provides a molecule comprising an antibody fragment which is capable of binding to a RapR7 protein.

The invention also provides an isolated nucleic acid comprising a fragment of a mammalian RapR7 gene consisting of at least 8 nucleotides. In a preferred embodiment, the invention provides an isolated nucleic acid comprising a fragment of a mammalian RapR7 gene comprising any one of exons 1-11 of a mammalian RapR7 gene. In another preferred embodiment, the invention provides an isolated nucleic acid comprising a fragment of a mammalian RapR7 gene comprising an intron, or a fragment thereof, of a mammalian RapR7 gene. The invention also provides an isolated nucleic acid comprising a nucleotide sequence encoding a fragment of a mammalian RapR7 protein that displays one or more functional activities of the mammalian RapR7 protein. The invention further provides an isolated nucleic acid comprising a nucleotide sequence encoding any one of the fragments of a RapR7 protein as set forth in the invention.

The invention also provides a recombinant cell containing a nucleic acid comprising a nucleotide sequence encoding a fragment of a mammalian RapR7 protein that displays one or more functional activities of the mammalian RapR7 protein.

The invention also provides methods of production of proteins encoded by a RapR7 gene, and derivatives and analogs thereof. In a preferred embodiment, the invention provides a method of producing a mammalian RapR7 protein comprising: (a) growing a recombinant cell containing a nucleic acid encoding a fragment of a mammalian RapR7 protein that displays one or more functional activities of the mammalian RapR7 protein such that said encoded fragment of said RapR7 protein is expressed by the cell; and (b) recovering the expressed fragment of the mammalian RapR7 protein. The invention also provides the product of the method.

The invention also provides pharmaceutical composition comprising a therapeutically effective amount of a mammalian RapR7 protein or a fragment thereof and a pharmaceutically acceptable carrier. The RapR7 fragment of the invention can be of any size having the desired activity, e.g., 5, 10, 20, 50, 100, or 200 amino acids. The invention also provides a pharmaceutical composition comprising a therapeutically effective amount of an antibody capable of binding to a mammalian RapR7 protein and a pharmaceutically acceptable carrier.

The present invention also provides methods for generating a genetically modified cell having altered sensitivity to rapamycin. In one embodiment, the method comprises introducing randomly into the genome of a host cell of a selected cell type of an organism a DNA construct which comprises (i) a regulated promoter and (ii) a selection marker coding sequence under the control of the regulated promoter; hereinafter, this DNA construct may be referred to as a “knockout construct.” The regulated promoter, when activated, initiates RNA transcription to produce an RNA and genetically modified cells exhibiting the desired phenotype are selected, e.g., if the host cell are rapamycin resistant, the selected cell is rapamycin sensitive, or if the host cell is rapamycin sensitive, the selected cell is rapamycin resistant. In a preferred embodiment, the method further comprises, prior to the step of introducing the knockout DNA construct, introducing into the genome of cells of the selected cell type a DNA construct encoding a transactivator, which comprises (i) a promoter and (ii) a nucleotide sequence encoding the transactivator under the control of the promoter and the genetically modified cell is generated by introducing the knockout DNA construct into a cell comprising a DNA construct encoding a transactivator which can activate the regulated promoter. In a preferred embodiment, the regulated promoter is a tetracycline regulated promoter and the transactivator activates the regulated promoter in the absence of tetracycline. In another preferred embodiment, the regulated promoter is a tetracycline regulated promoter and the transactivator activates the regulated promoter in the presence of tetracycline.

The knockout DNA construct may further comprise a rapid cloning element which comprises a replication origin sequence comprising sequences for initiation of replication and segregation, e.g., an Ori, and a bacterial selection marker, e.g., a chloramphenicol resistance gene. In one embodiment, the method of the invention further comprises activating the regulated promoter and identifying the genetically modified cell by a method comprising identifying a change in rapamycin resistance in the genetically modified cell. In another embodiment, the method further comprises cloning a fragment of genomic sequence by a method comprising: (a) obtaining a nucleotide sequence comprising the rapid cloning element and the fragment of genomic sequence; (b) circularizing the nucleotide sequence to generate a circular plasmid; and (c) transforming a suitable host cell using the circular plasmid. The sequence of the fragment of genomic sequence can be determined by a method comprising sequencing the circular plasmid. The location of the fragment of genomic sequence can be determined by a method comprising comparing the sequences with the genomic sequence of the selected cell type.

In the methods, the host cell can be but is not limited to a human host cell or a murine host cell, whereas the selected cell type can be a rapamycin resistant cell type or a rapamycin sensitive cell type. In a preferred embodiment, the cell type is a murine N2a cell line. In another preferred embodiment, the knockout DNA construct is integrated at a RapR7 locus. The engineered cells having the desired rapamycin phenotype can be used to screen or identify compounds that regulate rapamycin resistance.

A particular embodiment of the invention relates to a method for treating a mammal, e.g., a human, having a cancer which is caused by defective regulation of a RapR7 gene and/or defective activity of a protein encoded by the RapR7 gene. This aspect of the invention is based, in part, on the applicant's discovery that RapR7 is a cellular constituent that regulates rapamycin resistance. In particular, the data presented herein show that overexpression of RapR7 confers resistance to rapamycin. Thus, therapeutic regimens which downregulate expression or activity of RapR7 can be used to potentiate the effects of rapamycin; in particular, the antitumor effects of rapamycin. In one embodiment, the method comprises administering to the mammal a therapeutically sufficient amount of an agent which regulates the expression of the RapR7 gene and/or activity of the protein encoded by the RapR7 gene. In one embodiment, the cancer is caused by an increase of expression of the RapR7 gene, and the method comprises administering an agent which reduces the expression of the RapR7 gene in cells of the cancer. In another embodiment, the cancer is caused by a mutation in the RapR7 gene, and the method comprises administering an agent causes the expression of a normal version of the RapR7 gene in cells of the cancer. In still another embodiment, the method comprises administering an agent which comprises a RapR7 protein or a therapeutically equivalent fragment thereof.

The invention also provides a method for treating a mammal having a cancer, comprising administering to the mammal undergoing a rapamycin therapy a therapeutically sufficient amount of an agent which regulates the expression of a RapR7 gene and/or activity of a protein encoded by the RapR7 gene such that rapamycin resistance is regulated. In a specific embodiment, the invention provides a method for treating a mammal having a cancer, comprising administering to the mammal i) a therapeutically sufficient amount of an agent which regulates the expression of a RapR7 gene and/or activity of a protein encoded by the RapR7 gene such that rapamycin resistance is regulated, and ii) a therapeutically sufficient amount of rapamycin or an analog or derivative of rapamycin. Preferably, the agent reduces the expression of the RapR7 gene in cells of the cancer. In another embodiment, the agent causes the expression of a normal version of the RapR7 gene in cells of the cancer. In still another embodiment, the agent comprises a RapR7 protein or a therapeutically equivalent fragment thereof.

The invention also provides methods for diagnosing in a mammal a cancer which is a result of defective regulation of a RapR7 gene or a predisposition to such a cancer. In one embodiment, the method comprises determining an expression level of the RapR7 gene in cells of the mammal, in which an expression level above a predetermined threshold level indicates that the mammal has or is predisposed of the cancer. In a preferred embodiment, the expression level of the RapR7 gene is determined by a method comprising measuring the expression level of the RapR7 gene using one or more polynucleotide probes, each of which comprises a nucleotide sequence in the RapR7 gene. In one embodiment, the one or more polynucleotide probes comprise at least one polynucleotide probe comprising a nucleotide sequence within one of exons 1-11 of the RapR7 gene. In another embodiment, the one or more polynucleotide probes comprise at least one polynucleotide probe comprising a nucleotide sequence within an intron of the RapR7 gene. In particularly preferred embodiments of the invention, the methods is used to diagnose a cancer in a human. In one embodiment, the one or more polynucleotide probes comprise at least one polynucleotide probe comprising a nucleotide sequence comprised in the nucleotide sequence encoding a WD domain or a transmembrane domain in a RapR7 protein. Preferably, the one or more polynucleotide probes are polynucleotide probes on a microarray.

In another embodiment, the invention provides a method for diagnosing in a mammal a cancer which is a result of defective regulation of a RapR7 gene or a predisposition to such a cancer comprising determining a level of abundance of a protein encoded by the RapR7 gene in cells of the mammal, in which a level of abundance of the protein above a predetermined threshold level indicates that the mammal has or is predisposed of the cancer. In still another embodiment, the invention provides a method for diagnosing the cancer comprising determining a level of activity of a protein encoded by the RapR7 gene in cells of the mammal, in which an activity level above a predetermined threshold level indicates that the mammal has or is predisposed of the cancer. In particularly preferred embodiments of the invention, the methods is used to diagnose a cancer in a human. In one embodiment, the protein is a human RapR7 protein as depicted in SEQ ID NO:6 or 7. In other preferred embodiments of the invention, the methods is used to diagnose a cancer in a mouse. In one embodiment, the protein is a murine RapR7 protein as depicted in SEQ ID NO:3 or 4.

The invention also provides methods for evaluating rapamycin resistance in a cell. In one embodiment, the method comprises determining an expression level of a RapR7 gene in the cell, in which an expression level above a predetermined threshold level indicates that the cell is rapamycin resistant. In a preferred embodiment, the expression level of the RapR7 gene is determined by a method comprising measuring the expression level of the RapR7 gene using one or more polynucleotide probes, each of which comprises a nucleotide sequence in the RapR7 gene. In one embodiment, the one or more polynucleotide probes comprise at least one polynucleotide probe comprising a nucleotide sequence within one of exons 1-11 of the RapR7 gene. In another embodiment, the one or more polynucleotide probes comprise at least one polynucleotide probe comprising a nucleotide sequence within an intron of the RapR7 gene. In particularly preferred embodiments of the invention, the methods is used to evaluate rapamycin resistance in a human cell. In one embodiment, the one or more polynucleotide probes comprise at least one polynucleotide probe comprising a nucleotide sequence comprised in the nucleotide sequence encoding a PHD domain, a coiled-coil domain, a second peroximal domain, a nuclear localization domain, or a low complexity domain in a RapR7 protein. Preferably, the one or more polynucleotide probes are polynucleotide probes on a microarray.

In another embodiment, the invention provides a method for evaluating rapamycin resistance in a cell comprising determining a level of abundance of a protein encoded by a RapR7 gene in the cell, in which a level of abundance of the protein above a predetermined threshold level indicates that the cell is rapamycin resistant. In still another embodiment, the invention provides a method for evaluating rapamycin resistance in a cell comprising determining a level of activity of a protein encoded by the RapR7 gene in cells of the mammal, in which an activity level above a predetermined threshold level indicates that the cell is rapamycin resistant. In particularly preferred embodiments of the invention, the methods is used to evaluating rapamycin resistance in a human cell. In one embodiment, the protein is a human RapR7 protein as depicted in SEQ ID NO:6 or 7. In other preferred embodiments of the invention, the methods is used to evaluating rapamycin resistance in a murine cell. In one embodiment, the protein is a murine RapR7 protein as depicted in SEQ ID NO:3 or 4.

The present invention also provides a method for regulating rapamycin resistance in a cell. In one embodiment, the method comprises contacting the cell with a sufficient amount of an agent which regulates the expression of a RapR7 gene and/or the activity of a protein encoded by the RapR7 gene such that rapamycin resistance is regulated. The invention also provides methods for regulating rapamycin resistance in a mammal, comprising administering to the mammal a therapeutically sufficient amount of an agent which regulates the expression of a RapR7 gene and/or the activity of a protein encoded by the RapR7 gene such that rapamycin resistance is regulated. The invention further provides a method for regulating the growth of a cell, comprising contacting the cell with i) a sufficient amount of an agent which regulates the expression of a RapR7 gene and/or the activity of a protein encoded by the RapR7 gene such that rapamycin resistance is regulated; and ii) a sufficient amount of rapamycin or an analog or derivative of rapamycin. Preferably, the agent reduces the expression of the RapR7 gene in the cell. In another embodiment, the agent causes the expression of a normal version of the RapR7 gene in the cell. In still another embodiment, the agent comprises a RapR7 protein or a therapeutically equivalent fragment thereof.

The invention also provides a method of identifying an agent that is capable of regulating rapamycin resistance via its capability of modulating the expression of a RapR7 gene and/or the activity of a protein encoded by the RapR7 gene. In one embodiment, the method comprises comparing the inhibitory effects of rapamycin on cells expressing the RapR7 gene in the presence of the agent and in the absence of the agent, and identifying the agent as capable of regulating rapamycin resistance if there is a difference in the inhibitory effects of rapamycin. In a specific embodiment, the invention provides a method comprising: (a) contacting a first cell expressing the RapR7 gene with rapamycin in the presence of the agent and measuring a first growth inhibitory effect; (b) contacting a second cell expressing the RapR7 gene with rapamycin in the absence of the agent and measuring a second growth inhibitory effect; and (c) comparing the first and second inhibitory effects as measured in step (a) and (b), and identifying the agent as capable of regulating rapamycin resistance if there is a difference between the first and second inhibitory effects. Preferably, the agent reduces the expression of the RapR7 gene in the cell. In another embodiment, the agent causes the expression of a normal version of the RapR7 gene in the cell. In still another embodiment, the agent comprises a RapR7 protein or a therapeutically equivalent fragment thereof.

The invention further provides methods of producing an antibody that binds specifically to a RapR7 protein. In one embodiment, the method comprises raising the antibody against the RapR7 protein or a polypeptide comprising an fragment of the RapR7 protein. In one embodiment, the protein is a human RapR7 protein as depicted in SEQ ID NO:6 or 7. In other preferred embodiments of the invention, the RapR7 protein used to produce the antibody is a murine RapR7 protein, e.g., the murine RapR7 protein as depicted in SEQ ID NO:3 or 4.

The invention further provides an antibody that binds specifically to a RapR7 protein or a fragment of the RapR7 protein such that binding of the antibody to the RapR7 protein regulates rapamycin resistance. In one embodiment, the antibody binds specifically to a human RapR7 protein. In another embodiment, the antibody binds specifically to a human RapR7 protein. In still another embodiment, the antibody binds specifically to a murine RapR7 protein.

The invention further provides an agent that regulates the expression of a RapR7 gene such that rapamycin resistance is regulated. In one embodiment, the agent comprises a molecule which regulates the expression of the RapR7 gene. In a preferred embodiment, the molecule reduces expression of the RapR7 gene. In another preferred embodiment, the agent causes the expression of a normal version of the RapR7 gene in a cell.

The invention further provides a cell comprising at a RapR7 locus a knockout DNA construct which comprises (i) a regulated promoter and (ii) a selection marker coding sequence under the control of the regulated promoter. In a cell of the invention, activation of the regulated promoter initiates RNA transcription to produce an antisence RNA. In a preferred embodiment, the cell of the invention further comprises a DNA construct which comprises (i) a promoter and (ii) a nucleotide sequence encoding a transactivator which can activate the regulated promoter, in which the nucleotide sequence is under the control of the promoter. The cell of the invention can also comprise a rapid cloning element comprising a replication origin sequence comprising sequences for initiation of replication and segregation, e.g., an Ori, and a bacterial selection marker, e.g., a chloramphenicol resistance gene. Preferably, in the cell of the invention, the regulated promoter is a tetracycline regulated promoter, and the transactivator binds to the regulated promoter in the absence of tetracycline. The cell of the invention can be a rapamycin resistant cell type or a rapamycin sensitive cell type. The cell of the invention can also be a human or a murine cell. In a preferred embodiment, the cell is a murine N2a cell. In another preferred embodiment, the integration site is in the intron between exon 1 and 2 of the RapR7 locus. The invention further provides a kit for screening for agents which regulate rapamycin resistance and/or tumorgenesis, comprising in one or more containers (i) the cell of the invention; (ii) tetracycline or a derivative or analog thereof; and (iii) rapamycin or a derivative or analog thereof.

The invention further provides DNA microarrays for diagnosing rapamycin resistance. The microarray of the invention comprises one or more polynucleotide probes, each of which comprises a nucleotide sequence in a RapR7 gene. In one embodiment, the one or more polynucleotide probes comprise at least one polynucleotide probe comprising a nucleotide sequence within one of exons 1-11 of a RapR7 gene. In another embodiment, the one or more polynucleotide probes comprise at least one polynucleotide probe comprising a nucleotide sequence within an intron of the RapR7 gene. In still another embodiment, the one or more polynucleotide probes comprise at least one polynucleotide probe comprising a nucleotide sequence comprised in the nucleotide sequence encoding a PHD domain, a coiled-coil domain, a second peroximal domain, a nuclear localization domain, or a low complexity domain in a human RapR7 protein.

The invention further provides a kit for diagnosis of rapamycin resistance, comprising in one or more containers one or more polynucleotide probes, wherein each of the polynucleotide probes comprises a nucleotide sequence in a RapR7 gene.

### 4. BRIEF DESCRIPTION OF THE FIGURES

FIG. 1A depicts a nucleotide sequence (SEQ ID NO:1) flanking the 5′ side of the RHKO insertion site. The sequence was obtained from RHKO clone RapR7. FIG. 1B depicts alignment of sequences obtained from two RHKO clones RapR71 and RapR72.

FIGS. 2A-2B depict the cDNA sequence of the murine RapR7 gene (SEQ ID NO:2) and the encoded amino acid sequence of the murine RapR7 protein (SEQ ID NO:3). FIG. 2B also shows a fragment of the murine RapR7 protein (SEQ ID NO:4).

FIGS. 3A-3E depict the cDNA sequences and the translated amino acid sequences and the regulatory sequences of human RapR7. FIGS. 3A-3B: the cRNA sequence (SEQ ID NO:5, FIG. 3A) and the amino acid sequence (SEQ ID NO:6, FIG. 3B) of human RapR7. FIG. 3B also shows a fragment of the human RapR7 protein (SEQ ID NO:7). FIG. 3C depicts the nucleotide sequences of exons 1-11 (SEQ ID NO:8 through SEQ ID NO:18) of the human RapR7 gene. FIG. 3D depicts a regulatory sequence region of human RapR7 (SEQ ID NO:19). FIG. 3E depicts promoter sequences of human RapR7 (SEQ ID NOS:20-23).

FIG. 4A depicts an alignment of the cDNA sequences of the human and murine RapR7 genes. FIG. 4B depicts an alignment of the human and murine RapR7 protein sequences. M: murine; H: human.

FIG. 5A depicts the genomic region of the murine RapR7 gene. FIG. 5B depicts the location of the RHKO insertion site in the murine RapR7 gene. The RHKO vector is inserted in the intron between exon 1 and exon 2 of the murine RapR7 gene.

FIG. 6A depicts the genomic region of the human RapR7 gene. FIG. 6B depicts the chromosomal location of the human RapR7 gene. FIGS. 6C-6D depict the exon structure of the human RapR7 gene. 6C: cDNA sequence and exon structure; 6D: exon information; and 6E: splice site information. FIG. 6F depicts alternative splice variants. FIG. 6G depicts identified PHD domains in the human RapR7 gene. FIG. 6H depicts the structure of a PHD domain. PHD-finger (Pfam Accession number: PF00628) folds into an interleaved type of Zn-finger chelating 2 Zn ions in a similar manner to that of the RING and FYVE domains. The PHD finger [MEDLINE:95216093], [PUB00005675] is a C4HC3 zinc-finger-like motif found in nuclear proteins thought to be involved in chromatin-mediated transcriptional regulation. The PHD finger motif is reminiscent of, but distinct from, the C3HC4 type RING finger. The function of this domain is not yet known, but it is believed to be analogous to the LIM domain, i.e., it is involved in protein-protein interaction and is important for the assembly or activity of multicomponent complexes involved in transcriptional activation or repression. Also similar to the RING finger and the LIM domain, the PHD finger is thought to bind two zinc ions.

FIG. 7 illustrates sensitivity of the N2a cell line to the growth inhibitory effect of rapamycin. The growth of N2a cells is completely inhibited when the cells are treated with rapamycin at a concentration of 1 μM or greater.

FIGS. 8A-8B illustrate reversible inhibition by rapamycin in RapR7 clone (MTT proliferation assay). 8A: Solid bar, measurement when the knockout construct is expressed; shaded bar, measurement when the expression of the knockout construct is suppressed; and open bar, control. 8B: Calculated reversibility R according to Equation 1. FIG. 8C illustrates RapR7 colony after 12 days of infection with an RHKO gene search vector.

FIGS. 8D-8E illustrate markers of G1 arrest in RapR7 clone. Colony 6 is the RapR7 clone of the present invention. Colonies 1, 2, and 8 are clones which do not exhibit rapamycin resistance.

FIGS. 9A-9B depict exemplary knockout or gene search constructs. FIG. 9C depicts the retroviral vector used to introduce the knockout construct. TRE: Tetracycline Response Element. FIG. 9D depicts a construct which expresses the tetracycline-controlled transactivator (tTA) of a Tet-off system.

FIG. 10 depicts exemplary SNPs in human RapR7 gene (NCBI dbSNP Site: www.ncbi.nlm.nih.gov/SNP/index.html).

FIGS. 11A-11C illustrate alternative splicing information and protein analysis data of the human RapR7 gene.

### 5. DETAILED DESCRIPTION OF THE INVENTION

The invention provides nucleotide sequence of a novel mammalian gene which is involved in rapamycin resistance and tumorgenesis, the RapR7 gene, and amino acid sequences of its encoded proteins, and derivatives and analogs thereof. The invention provides derivatives and analogs of a protein encoded by the RapR7 gene. The invention provides fragments (and derivatives and analogs thereof) of a mammalian RapR7 gene product. The invention also provides methods of production of mammalian RapR7 gene products, and derivatives and analogs thereof.

The present invention provides methods for identifying cellular constituents, e.g., genes and proteins, and/or pathways that are involved in rapamycin resistance and/or tumorgenesis. The invention also provides methods for generating genetically modified cells having altered sensitivity to rapamycin by knocking out a gene which mediates rapamycin resistance. The present invention also provides methods and compositions for regulating rapamycin resistance and/or tumorgenesis by modulating such cellular constituents and/or pathways. In specific embodiments of the invention, the present invention provides mammalian RapR7 genes and proteins and derivatives and analogs thereof, fragments (and derivatives and analogs thereof) of a mammalian RapR7 protein, and methods for production of mammalian RapR7 proteins (and derivatives and analogs thereof). The invention also provides methods and compositions for regulating rapamycin resistance and/or tumorgenesis in a cell or organism by targeting RapR7 gene and/or protein. In the methods of the invention, rapamycin resistance and/or tumorgenesis is regulated, e.g., inhibited, reduced or enhanced, by modulating the expression of RapR7 gene and/or the activity, e.g., the interaction of RapR7 gene with other intra- or extra-cellular molecules. The compositions of the invention include but not limited to nucleic acid encoding RapR7 gene and homologues, analogues, and deletions thereof, as well as antisense, ribozyme, triple helix, double-stranded RNA, antibody and polypeptide molecules, and small organic or inorganic molecules. The invention also provides methods and compositions for treatment of diseases, e.g., cancers, by modulating the activity of RapR7 gene in conjunction with a rapamycin therapy. The invention also provides methods and compositions for diagnosing and screening RapR7 mediated rapamycin resistance and/or tumorgenesis in patients. The invention further provides host cells whose RapR7 gene can be reversibly activated or overexpressed, i.e., overexpression or activation can be switched on and off, and methods of using RapR7 gene in evaluation and screening for drugs which modulate rapamycin resistance and/or tumorgenesis, and methods of identifying the functions of RapR7 gene and cellular pathways of RapR7 gene.

The invention, is based, in part, on the identification of the involvement of RapR7 gene in rapamycin resistance and tumorgenesis using the random homozygous knockout (RHKO) method. RHKO clones whose RapR7 gene can be activated or overexpressed reversibly exhibit resistance to rapamycin when RapR7 gene is activated or overexpressed and sensitivity to rapamycin when the gene is not activated or overexpressed.

In the disclosure, resistance to rapamycin is often referred to. It will be apparent to a skilled person in the art that the disclosure is equally applicable to other rapamycin derivatives or analogs, such as, but not limited to, the rapamycin ester CCI-779.

For simplicity of discussion, the invention is described in the subsections below by way of examples for the huamn and murine RapR7 genes. However, the principles may be analogously applied to RapR7 genes of other species.

### 5.1. DNA Encoding RapR7

The present invention provides a novel mammalian gene, the RapR7 gene. RapR7 gene is identified as a gene involved in rapamycin resistance and tumorgenesis. The present invention also provides recombinant mammalian DNA molecules, cloned genes, or degenerate variants thereof, of a RapR7 gene which may involve in rapamycin resistance and tumorgenesis in a cell or organism.

### 5.1.1. THE RapR7 Gene

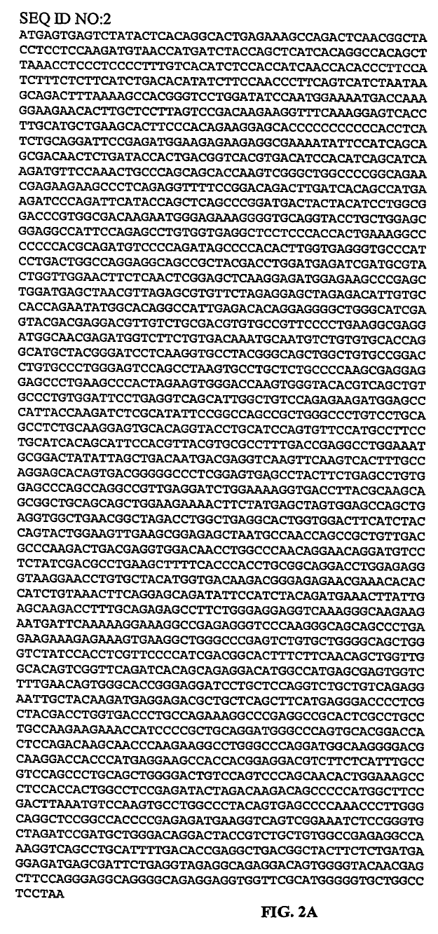

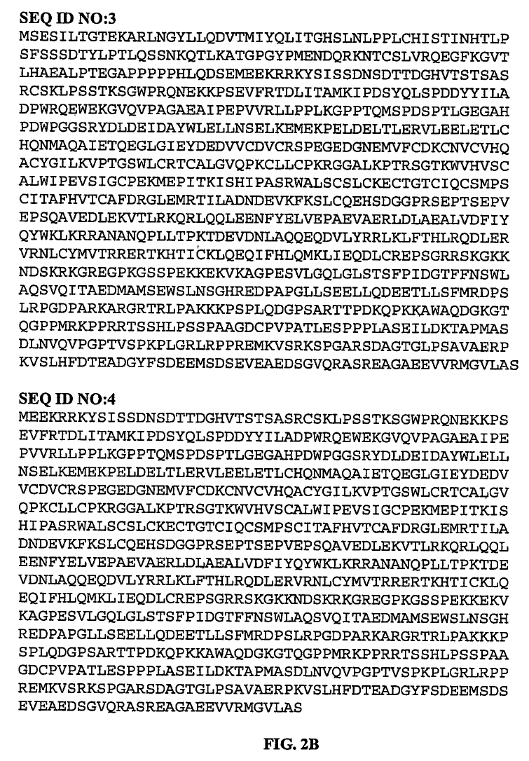

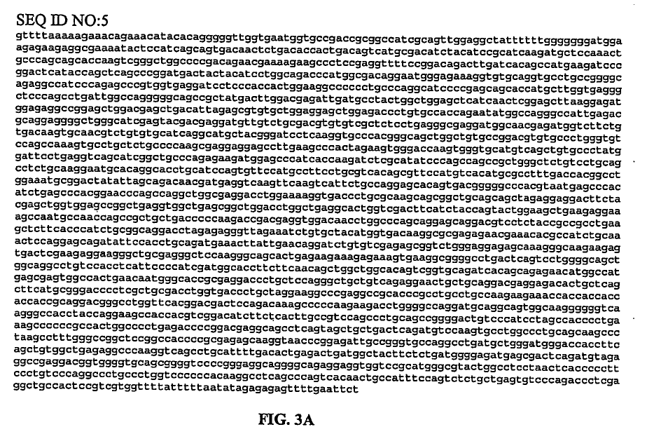

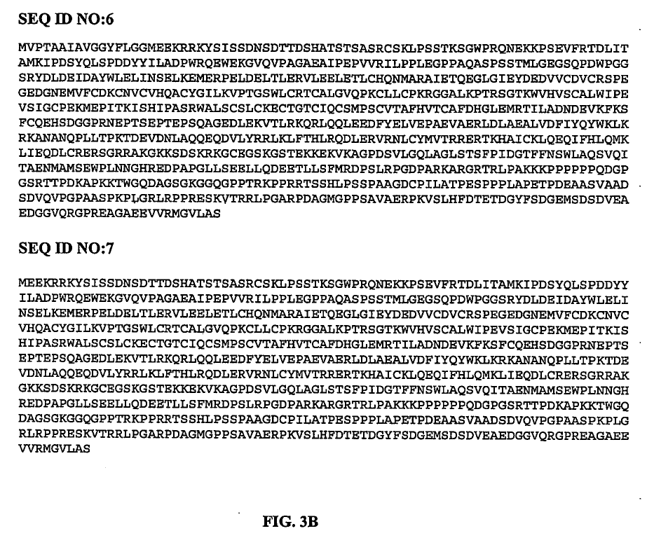

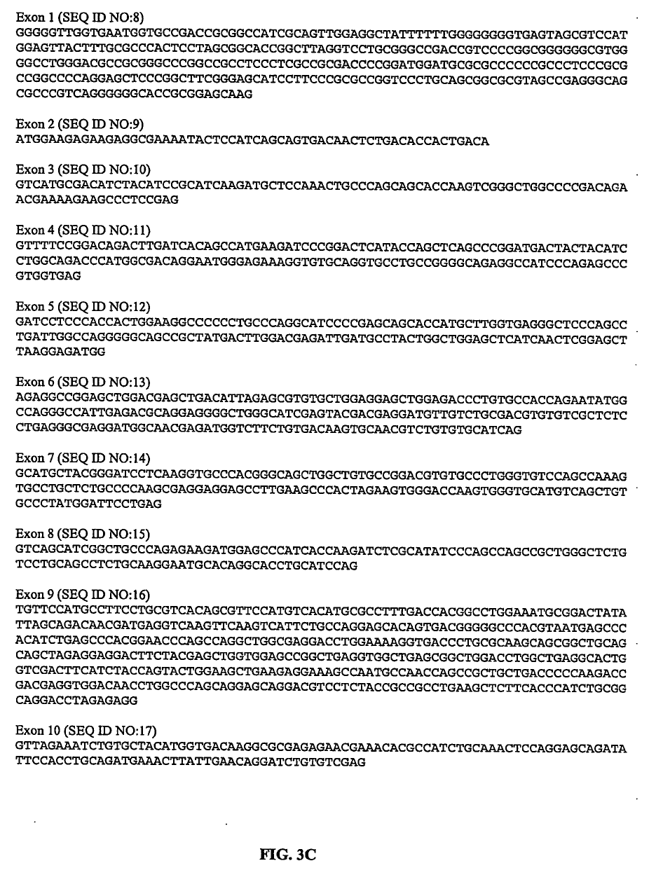

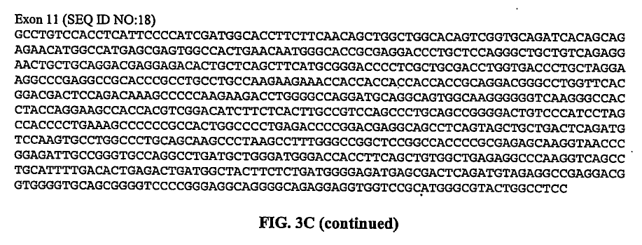

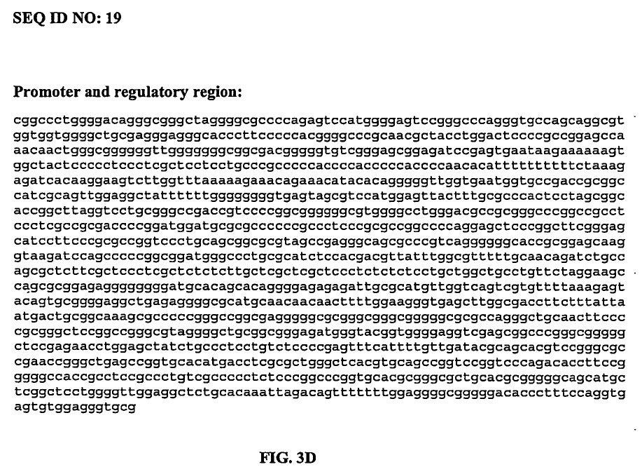

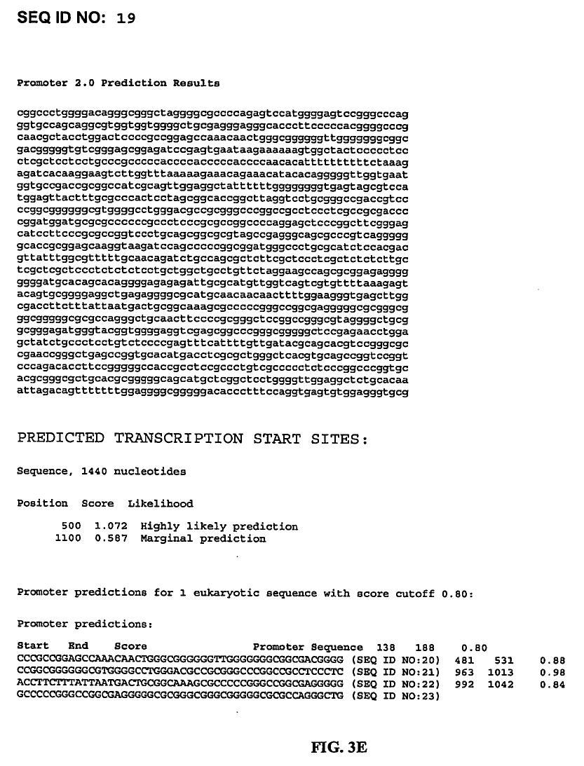

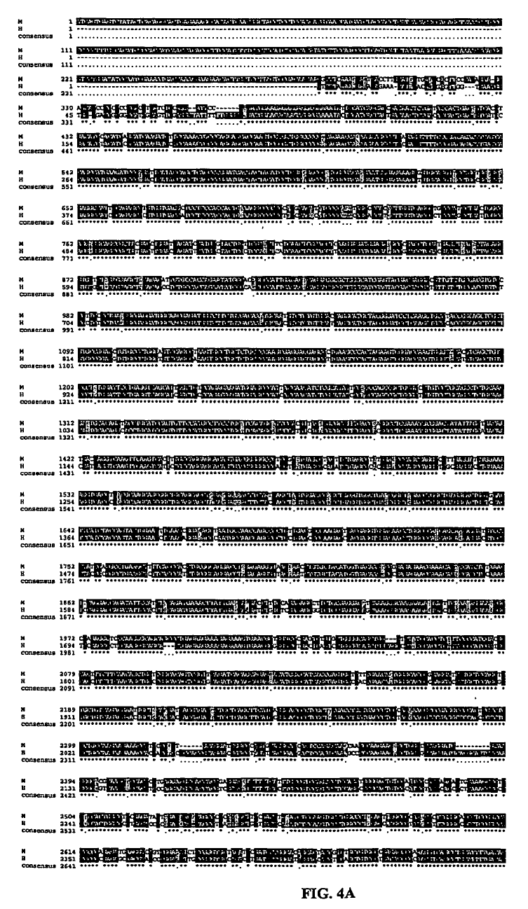

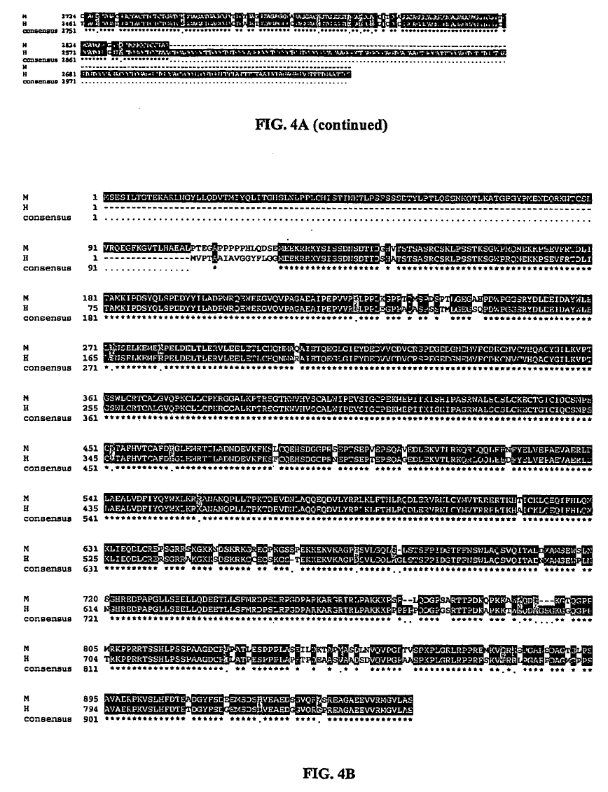

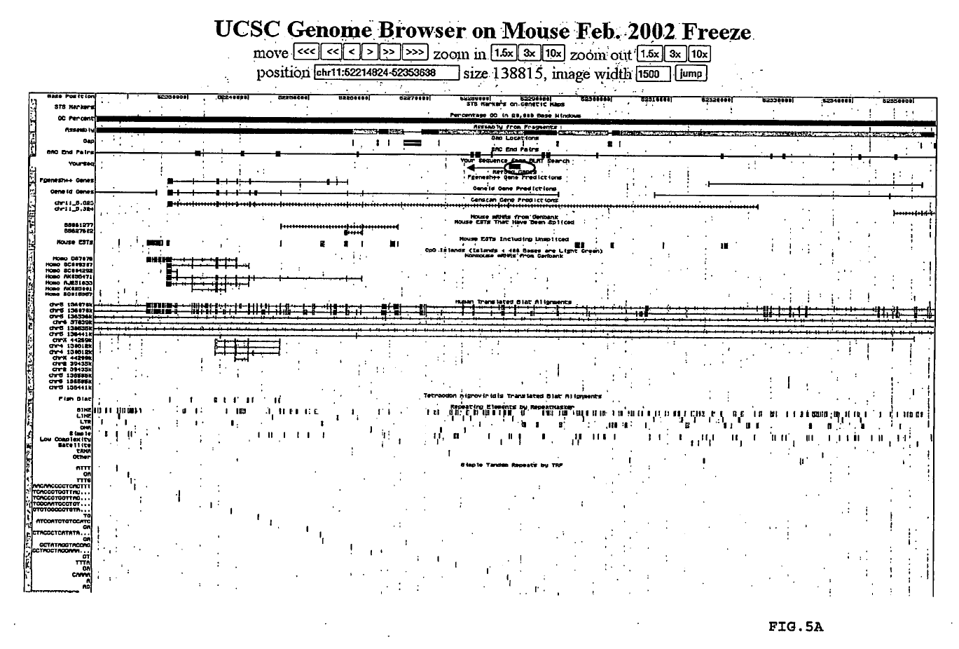

As used herein, “RapR7 gene” includes the genomic portion of DNA which is transcribed by RNA polymerase and encodes one or more RapR7 proteins. The RapR7 gene may include a 5′ untranslated region (“UTR”), introns, exons and a 3′UTR and regulatory sequences. The mRNA sequences of murine and human RapR7 genes and the encoded proteins are shown in FIGS. 2A-2B (SEQ ID NOS:2-3) and FIGS. 3A-3B (SEQ ID NOS:5-6), respectively.

In preferred embodiments, the invention provides (a) a gene comprising the DNA sequence shown in FIGS. 2A and 3A, or comprised in the RHKO clone RapR7 in which a DNA construct is inserted in the intron between exon 1 and 2; (b) any DNA sequence that encodes the amino acid sequence shown in FIGS. 2B and 3B, or encoded by the gene in the RHKO clone RapR7 in which a DNA construct is inserted in the intron between exon 1 and 2; (c) any DNA sequence that hybridizes to the complement of the DNA sequences that encode the amino acid sequence shown in FIGS. 2B and 3B, or encoded by the gene in the RHKO clone RapR7 in which a DNA construct is inserted in the intron between exon 1 and 2, under highly stringent conditions, e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (Ausubel F. M. et al., eds., 1989, Current Protocols in Molecular Biology, Vol. I, Green Publishing Associates, Inc., and John Wiley & sons, Inc., New York, at p. 2.10.3) and encodes a gene product functionally equivalent to a RapR7 gene product encoded by sequences shown in FIGS. 2A and 3A; and/or (d) any DNA sequence that hybridizes to the complement of the DNA sequences that encode the amino acid sequence shown in FIGS. 2B and 3B, or encoded by the gene in the RHKO clone RapR7 in which a DNA construct is inserted in the intron between exon 1 and 2, under less stringent conditions, such as moderately stringent conditions, e.g. washing in 0.2×SSC/0.1% SDS at 42° C. (Ausubel et al., 1989, supra), yet which still encodes a functionally equivalent RapR7 gene product. As used herein, RapR7 gene also includes degenerate variants of DNA sequences SEQ ID NO: 2 or 5.

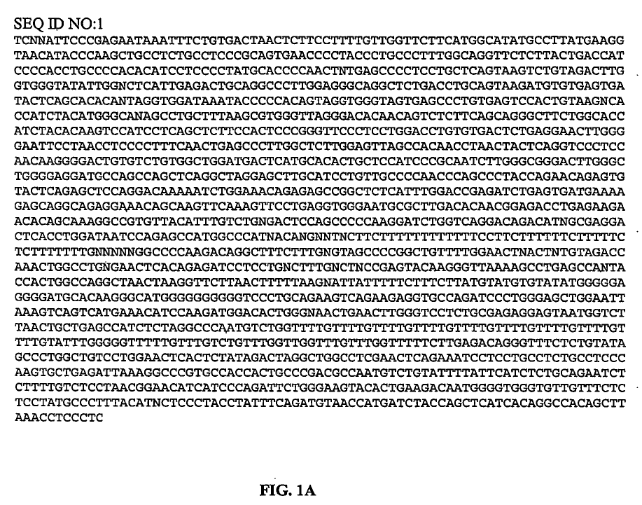

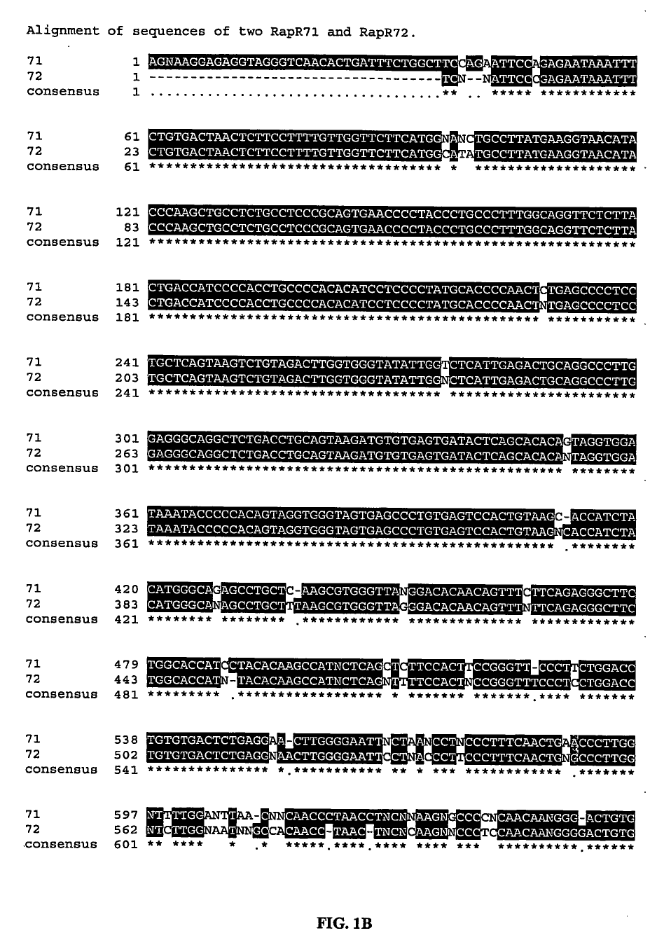

The invention also provides exon and intron sequences comprised in a RapR7 gene. In one embodiment, the invention provides exons 1-11 of the human RapR7 gene (SEQ ID NOS:8-18). In another embodiment, the invention provides the genomic sequence of a RapR7 gene comprised in a 4 kb plasmid carrying a 1.4 kb genomic DNA flanking the RHKO insertion site obtained by digestion of the RapR7 genomic DNA using HindIII. Sequences of two such plasmids (designated as RapR71 and RapR72, respectively) are depicted in FIG. 1B.

The invention also provides regulatory sequences of a RapR7 gene. In one embodiment, the invention provides a regulatory sequence of a human RapR7 gene (SEQ ID NO:19). In another embodiment, the invention provides promoter sequences of human RapR7 gene (SEQ ID NOS:20-23).

The invention also includes portions of a RapR7 gene, e.g., a portion encoding a fragment of a RapR7. In a preferred embodiment, the invention provides a sequence flanking the insertion site of the knockout construct as illustrated in FIG. 1 (SEQ ID NO:1). In another preferred embodiment, the invention provides a fragment of a RapR7 gene comprising the nucleotide region encoding a fragment comprising the RapR7 protein fragment as described by SEQ ID NO:4 or 7. In one embodiment, the invention provides a fragment of a RapR7 gene comprising the nucleotide region encoding a fragment comprising a PHD domain of a RapR7 gene product. In a specific embodiment, the invention provides a fragment of a human RapR7 gene comprising the nucleotide region encoding amino acids 217-263 or 326-381 of the human RapR7 protein, or its murine homolog. In another embodiment, the invention provides a fragment of a RapR7 gene comprising the nucleotide region encoding a fragment comprising a coiled-coil domain of the RapR7 gene product. In a specific embodiment, the invention provides a fragment of a human RapR7 gene comprising the nucleotide region encoding amino acids 163-190 of the human RapR7 protein, or its murine homolog. In another embodiment, the invention provides a fragment of a RapR7 gene comprising the nucleotide region encoding a fragment comprising a second peroximal domain of the RapR7 gene product. In a specific embodiment, the invention provides a fragment of a human RapR7 gene comprising the nucleotide region encoding amino acids 514-522 of the human RapR7 protein, or its murine homolog. In still another embodiment, the invention provides a fragment of a RapR7 gene comprising the nucleotide region encoding a fragment comprising a nuclear localization domain of the RapR7 gene product. In a specific embodiment, the invention provides a fragment of a human RapR7 gene comprising the nucleotide region encoding amino acids 20-23, 548-564, 549-565, 650-656, 661-667, or 663-666 of the human RapR7 protein, or its murine homolog. In another embodiment, the invention provides a fragment of a RapR7 gene comprising the nucleotide region encoding a fragment comprising a low complexity domain of the RapR7 gene product. In a specific embodiment, the invention provides a fragment of a murine RapR7 gene comprising the nucleotide region encoding amino acids 25-44, 177-193, 550-566, 661-671, 690-711, 727-741, or 744-753 of the human RapR7 protein, or its murine homolog. In still another embodiment, the invention provides a portion of a RapR7 gene encoding a fragment of a RapR7 protein which does not comprise a PHD, a coiled-coil, a second peroximal domain, a nuclear localization domain, or a low complexity domain of the RapR7 gene product, or a fragment of such a fragment. The invention also provides any sequence that is at least 30%, 50%, 70%, 90%, or 95% homologous to such fragments of a RapR7 gene.

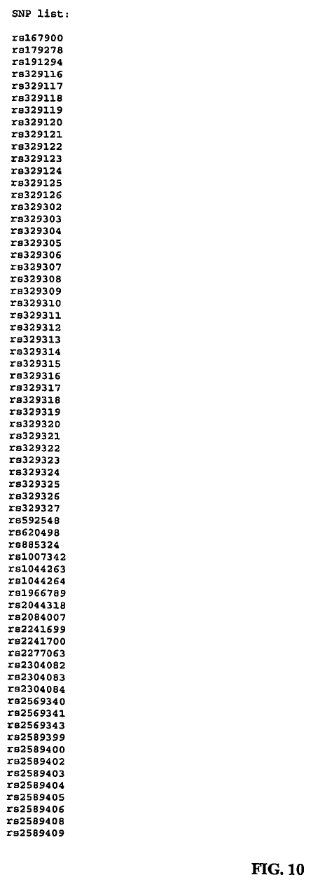

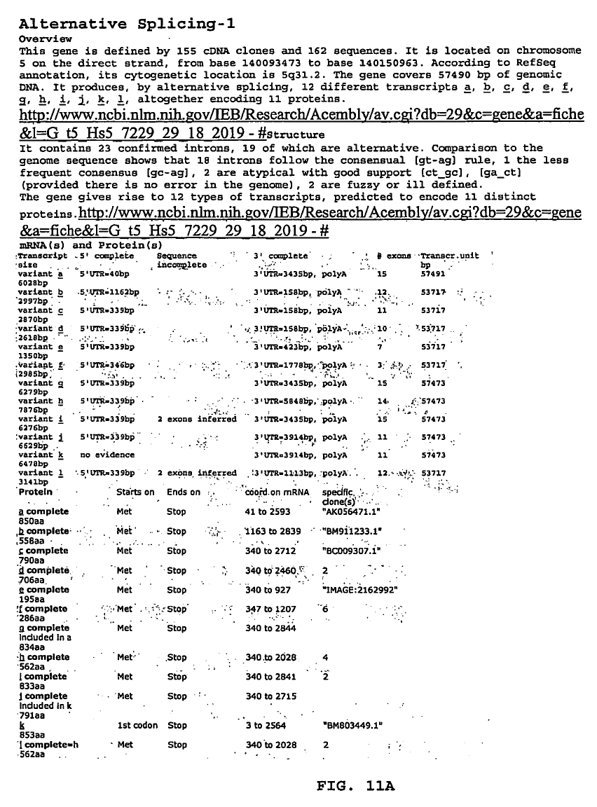

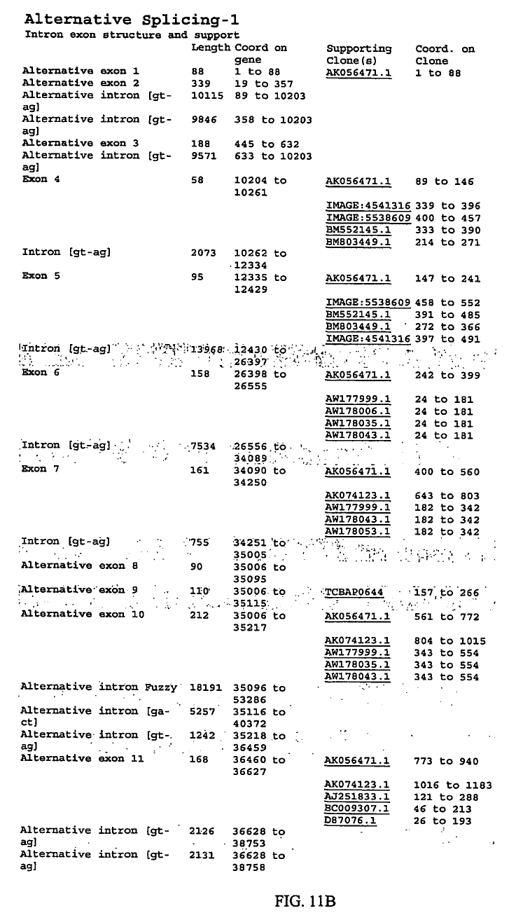

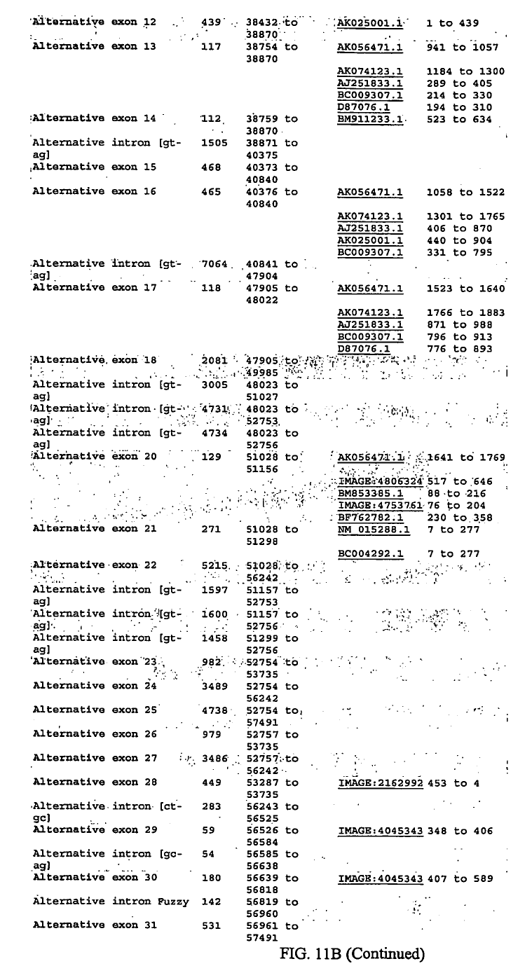

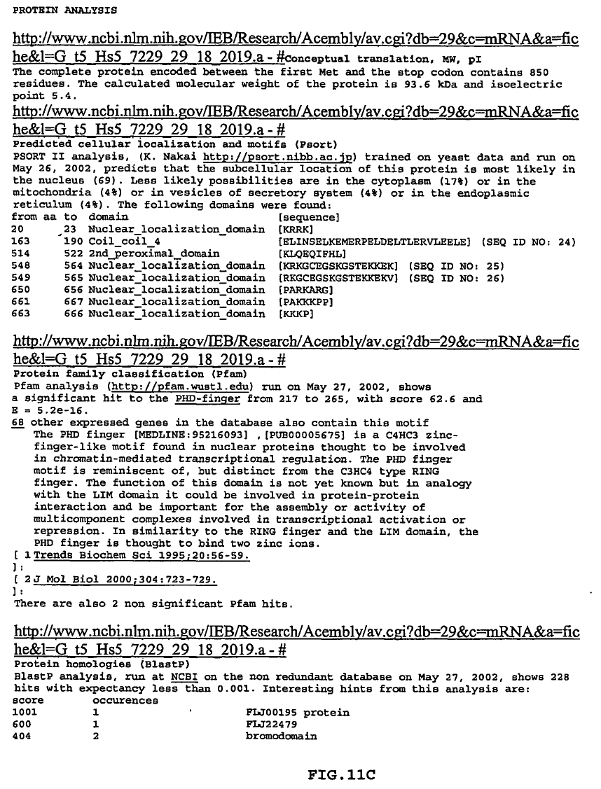

The invention also provides nucleotide sequences comprising mutations in a RapR7 gene which cause a change in the amino acid sequence of the encoded protein. Exemplary SNPs are illustrated in FIG. 10.

The invention also provides nucleotide sequences which are comprised in a RapR7 gene and are at least 20, 25, 40, 60, 80, 100, 500, 1000 bases in length. Such sequences may be useful as probe sequences for monitoring expression of a RapR7 gene. The invention also provides nucleotide sequences which are comprised in a RapR7 gene and are at least 20, 50, 100, 500, 1000, 2000, 5000 bases in length. Such sequences may be useful for production of RapR7 peptides.

The invention also includes nucleic acid molecules, preferably DNA molecules, that hybridize to, and are therefore the complements of, the DNA sequences described in the preceding paragraphs. Such hybridization conditions may be highly stringent or less highly stringent, as described above. In instances wherein the nucleic acid molecules are deoxyoligonucleotides (“oligos”), highly stringent conditions may refer, e.g., to washing in 6×SSC/0.05% sodium pyrophosphate at 37° C. (for 14-base oligos), 48° C. (for 17-base oligos), 55° C. (for 20-base oligos), and 60° C. (for 23-base oligos). These nucleic acid molecules may encode or act as RapR7 gene antisense molecules, useful, for example, in RapR7 gene regulation (for and/or as antisense primers in amplification reactions of RapR7 gene nucleic acid sequences. With respect to RapR7 gene regulation, such techniques can be used to regulate, for example, resistance to rapamycin. Further, such sequences may be used as part of ribozyme and/or triple helix sequences, also useful for RapR7 gene regulation. Still further, such molecules may be used as components of diagnostic methods whereby, for example, the presence of a particular RapR7 allele responsible for causing a RapR7 related disorder, such as rapamycin resistance and/or tumorgenesis, may be detected.

The invention also encompasses (a) DNA vectors that contain any of the foregoing RapR7 coding sequences and/or their complements (i.e., antisense); (b) DNA expression vectors that contain any of the foregoing RapR7 coding sequences operatively associated with a regulatory element that directs the expression of the coding sequences; and (c) genetically engineered host cells that contain any of the foregoing RapR7 coding sequences operatively associated with a regulatory element that directs the expression of the coding sequences in the host cell. As used herein, regulatory elements include but are not limited to inducible and non-inducible promoters, enhancers, operators and other elements known to those skilled in the art that drive and regulate expression. Such regulatory elements include but are not limited to the cytomegalovirus hCMV immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast α-mating factors. The invention includes fragments of any of the DNA sequences disclosed herein. In preferred embodiments, RapR7 coding sequences are obtained by isolating the sequences flanking the insertion site in a RHKO clone.

In addition to the RapR7 gene sequences described above, homologs of such sequences present in other species can be identified and readily isolated, by molecular biological techniques well known in the art. Further, there can exist genes at other genetic loci within the genome that encode proteins which have extensive homology to one or more domains of the RapR7 gene product. These genes can also be identified via similar techniques.

With respect to the cloning of a RapR7 gene homolog in a species (e.g., human), the isolated RapR7 gene sequences disclosed herein may be labeled and used to screen a cDNA library constructed from mRNA obtained from appropriate cells or tissues (e.g., human MDCK cells) derived from the organism (e.g., human) of interest. The hybridization conditions used should be of a lower stringency when the cDNA library is derived from an organism different from the type of organism from which the labeled sequence was derived.

Alternatively, the labeled fragment may be used to screen a genomic library derived from the organism of interest, again, using appropriately stringent conditions. Low stringency conditions are well known to those of skill in the art, and will vary predictably depending on the specific organisms from which the library and the labeled sequences are derived. For guidance regarding such conditions see, for example, Sambrook et al., 1989, Molecular Cloning, A Laboratory Manual, Cold Springs Harbor Press, N.Y.; and Ausubel et al., 1989, Current Protocols in Molecular Biology, Green Publishing Associates and Wiley Interscience, N.Y.

Further, a RapR7 gene homolog may be isolated from, for example, human nucleic acid, by performing PCR using two degenerate oligonucleotide primer pools designed on the basis of amino acid sequences within the RapR7 gene product disclosed herein. The template for the reaction may be cDNA obtained by reverse transcription of mRNA prepared from, for example, human or non-human cell lines or tissue known or suspected to express a RapR7 gene allele.

The PCR product may be subcloned and sequenced to ensure that the amplified sequences represent the sequences of a RapR7 gene nucleic acid sequence. The PCR fragment may then be used to isolate a full length cDNA clone by a variety of methods. For example, the amplified fragment may be labeled and used to screen a bacteriophage cDNA library. Alternatively, the labeled fragment may be used to isolate genomic clones via the screening of a genomic library.

PCR technology may also be utilized to isolate full length cDNA sequences. For example, RNA may be isolated, following standard procedures, from an appropriate cellular or tissue source (i.e., one known, or suspected, to express the RapR7 gene, such as, for example, epithelia). A reverse transcription reaction may be performed on the RNA using an oligonucleotide primer specific for the most 5′ end of the amplified fragment for the priming of first strand synthesis. The resulting RNA/DNA hybrid may then be “tailed” with guanines using a standard terminal transferase reaction, the hybrid may be digested with RNAase H, and second strand synthesis may then be primed with a poly-C primer. Thus, cDNA sequences upstream of the amplified fragment may easily be isolated. For a review of cloning strategies which may be used, see e.g., Sambrook et al., 1989, supra.

RapR7 gene sequences may additionally be used to isolate mutant RapR7 gene alleles. Such mutant alleles may be isolated from individuals either known or proposed to have a genotype which contributes to rapamycin resistance and/or tumorgenesis. Mutant alleles and mutant allele products may then be utilized in the therapeutic and diagnostic systems described below. Additionally, such RapR7 gene sequences can be used to detect RapR7 gene regulatory (e.g., promoter) defects which can affect RapR7 expression and/or activity.

A cDNA of a mutant RapR7 gene may be isolated, for example, by using PCR, a technique which is well known to those of skill in the art. In this case, the first cDNA strand may be synthesized by hybridizing an oligo-dT oligonucleotide to mRNA isolated from tissue known or suspected to be expressed in an individual putatively carrying the mutant RapR7 allele, and by extending the new strand with reverse transcriptase. The second strand of the cDNA is then synthesized using an oligonucleotide that hybridizes specifically to the 5′ end of the normal gene. Using these two primers, the product is then amplified via PCR, cloned into a suitable vector, and subjected to DNA sequence analysis through methods well known to those of skill in the art. By comparing the DNA sequence of the mutant RapR7 allele to that of the normal RapR7 allele, the mutation(s) responsible for the loss or alteration of function of the mutant RapR7 gene product can be ascertained.

Alternatively, a genomic library can be constructed using DNA obtained from an individual suspected of or known to carry the mutant RapR7 allele, or a cDNA library can be constructed using RNA from a tissue known, or suspected, to express the mutant RapR7 allele. The normal RapR7 gene or any suitable fragment thereof may then be labeled and used as a probe to identify the corresponding mutant RapR7 allele in such libraries. Clones containing the mutant RapR7 gene sequences may then be purified and subjected to sequence analysis according to methods well known to those of skill in the art.

Additionally, an expression library can be constructed utilizing cDNA synthesized from, for example, RNA isolated from a tissue known, or suspected, to express a mutant RapR7 allele in an individual suspected of or known to carry such a mutant allele. In this manner, gene products made by the putatively mutant tissue may be expressed and screened using standard antibody screening techniques in conjunction with antibodies raised against the normal RapR7 gene product, as described, below, in Section 5.3. (For screening techniques, see, for example, Harlow, E. and Lane, eds., 1988, “Antibodies: A Laboratory Manual”, Cold Spring Harbor Press, Cold Spring Harbor.) In cases where a RapR7 mutation results in an expressed gene product with altered function (e.g., as a result of a missense or a frameshift mutation), a polyclonal set of anti-RapR7 gene product antibodies are likely to cross-react with the mutant RapR7 gene product. Library clones detected via heir reaction with such labeled antibodies can be purified and subjected to sequence analysis according to methods well known to those of skill in the art.

### 5.1.2. Methods of Identifying a Gene Involved in Rapamycin Resistance and Tumorgenesis

The involvement of a gene in rapamycin resistance and/or tumorgenesis can be identified by introducing randomly into the genome or a suitable cell, e.g., an N2a cell, a DNA construct (i.e., the knockout construct) such that a gene is activated or inactivated, and screening for resultant clones which exhibit phenotypic changes in rapamycin resistance and/or tumorgenesis. Any mammalian cells include but are not limited to N2a, NT2, NT22, CHO, VERO, BHK, HeLa, COS, MDCK, 293, 3T3, WI38 can be used. Preferably, the cell line used is a rapamycin sensitive cell line, and the resultant clones are rapamycin resistant. Alternatively, a rapamycin resistant cell line can be used as the starting cell line, and the resultant clones are rapamycin sensitive.

In a preferred embodiment, a rapamycin resistance and/or tumorgenesis clone contains a gene which is knocked out by the random homozygous knockout (RHKO) process (see, e.g., U.S. Pat. Nos. 5,679,523; 5,807,995; 5,891,668; and 6,248,523; Li et al., 1996, Cell 85:319-329; PCT publication no. WO 03/027260, each of which is incorporated herein by reference in its entirety). In a RHKO clone, multiple alleles of a gene at a random chromosomal locus in the genome of a mammalian cell are inactivated concurrently. In another preferred embodiment, a rapamycin resistance and/or tumorgenesis clone contains an insertion of a suitable construct at a genomic locus such that the expression of a gene at the locus is activated or enhanced. In still another preferred embodiment, a rapamycin resistance and/or tumorgenesis clone contains an insertion of a suitable construct at a genomic locus such that a portion of a gene at the locus is overexpressed.

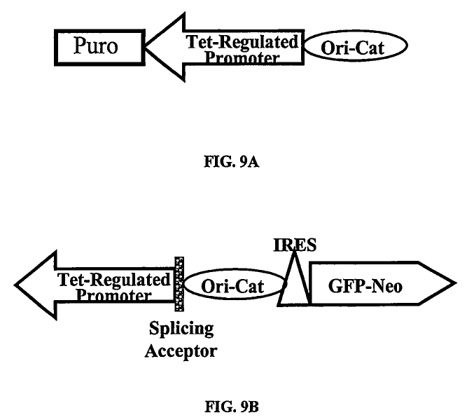

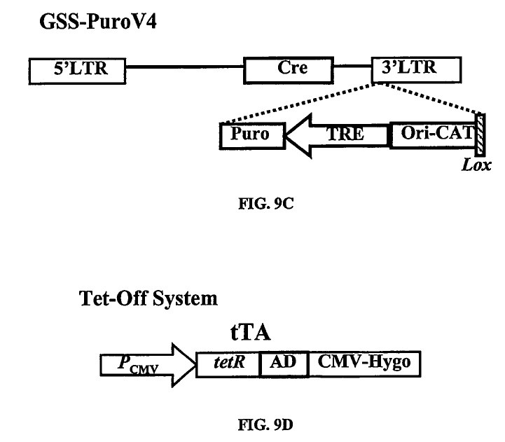

Preferably, a knockout construct (or gene search construct) comprising a selection marker sequence and a regulated promoter responsive to a transactivation factor and controlling the expression of the selection marker sequence is inserted into the genome of a selected cell line using a standard method known in the art, e.g., transefection or retroviral infection. In a preferred embodiment, a retroviral gene search vector comprising the knockout construct, viral genes and regulatory elements, and a Cre/Lox site specific recombination system is used to introduce the knockout construct into the genome of a cell (FIG. 9C). The Cre/Lox system allows the use of the retroviral elements for infection and the subsequent removal of the retroviral elements from chromosomal DNA after vector integration. Such a retroviral gene search vector allows highly efficient retroviral integration in target cells (up to 90%), and at the same time eliminate retroviral interference of selection marker gene activity and RNA transcription. The selection marker gene in the gene search vector can be a fusion gene consisting of a neomycin resistance gene and a bacterial β-galactosidase gene. In a preferred embodiment, the selection marker gene consists of a neomycin resistance gene or a puromycin resistance gene. In another preferred embodiment, the selection marker gene consists of a neomycin resistance gene or a puromycin resistance gene and a gene encoding a fluorescence protein such as a green fluorescence protein, e.g., a GFP-Neo or a GFP-Puro. Incorporation of fluorescence proteins allows direct detection of selection marker gene activity in living cells and direct isolation of positive cells by FACS (Fluorescence Activated Cell Sorter) without having to stain the cells. Preferably, the gene search vector further comprises a gene splicing element and/or an independent translation initiation signal. In one embodiment, a viral internal ribosomal entry site (IRES) is inserted into the selection marker gene to allow efficient translation in all three reading frames. More preferably, another DNA construct which includes a promoter, e.g., an SMV promoter, and a nucleotide sequence encoding the transactivator and under the control of the promoter is also inserted into the genome of the cells to allow production of the transactivator.

Preferably, the regulated promoter provides a rheostat genetic on/off switch. For example, either a Tet-on™ or a Tet-off™ system (Clontech, Palo Alto, Calif., see, e.g., http://www.clontech.com/products/literature/pdf/productlists/tetprodlist.pdf and http://www.clontech.com/products/literaturelpdf/brochures/TetBR.pdf, accessed on May 3, 2002) can be used for this purpose. In a preferred embodiment, a Tet-off™ system (Clontech) is used. In a Tet-off™ system the regulated promoter is a tetracycline regulated promoter which can be activated by a tetracycline regulated transactivator (see, e.g., Gossen et al, 1995, Science 268:1766-1769; Gossen et al., 1992, Proc. Natl. Acad. Sci. USA 89:5547-5551). The transactivator binds to tetracycline regulated promoter in the absence of tetracycline but not in the presence of tetracycline. Therefore, gene expression is kept off in the presence of tetracycline, whereas gene transcription is activated in the absence of tetracycline. Incorporation of a tetracycline regulation system into RHKO allows transcription of RNA, e.g., transcription of antisence RNA, to be turned on or off by removing or adding tetracycline in the cell culture medium, and also allows the rheostat regulation of RNA production by controlling the amount of tetracycline in the culture medium. This enables rapid validation of the antisense RNA effect by adding and removing tetracycline or a derivative or analog thereof, and determination of gene function under rheostat regulation.

The knockout construct can preferably contain a rapid cloning element comprising a bacterial plasmid replication origin, e.g., an Ori, and a bacterial selection marker, e.g., a chloramphenicol resistance gene for rapid and direct isolation of target genes. The chromosomal gene, the selection marker sequence, the plasmid replication origin, and the bacterial selection marker are transcribed as a single fusion messenger RNA, which is converted to double strand cDNA and circularized to a circular plasmid. The resulting circular plasmids are transformed into bacteria and rapidly amplified without additional gene cloning. The chromosomal genes are identified by sequencing the amplified plasmids. Alternatively the genomic DNA can be digested with restriction enzyme (such as Hind III, BamH1), recirculized by self-ligation and are transformed into bacteria and rapidly amplified. The genomic DNA flanking the knockout vector can be rapidly cloned directly by this strategy. Any bacterial plasmid replication origin, such as but not limited to Ori, colEI, pSC101, pUC, or f1 phage ori, can be used. Any bacterial selection markers, such as but not limited to, chloramphenicol, ampicillin, tetracycline, or kanamycin, can be used in the present invention.

In one embodiment, the construct is inserted into the open reading frame region of a genomic locus such that the transcription initiation sequence in the knockout construct is oriented for antisense RNA transcription in the direction away from the selection marker region sequence such that when activated by the transactivation factor, it initiates antisense RNA transcription extending from the knockout construct into the chromosomal locus flanking the knockout construct at its 5′ end. Thus, although only one allele of the gene is knocked out, antisense RNA transcripts inactivate the other allele or alleles. Cells contain the knockout construct, i.e., the RHKO clones, is selected based on the presence of activity of the selection marker. RHKO clones in which a gene involved in rapamycin resistance is inactivated are then subjected to rapamycin treatment, and rapamycin resistance RHKO clones are identified. In a preferred embodiment, the insertion site is in an exon of the gene. In another preferred embodiment, the insertion site is in an intron.

In another embodiment, the construct is inserted in front of an endogenous promoter such that the transcription initiation sequence in the knockout construct is oriented for activation or enhancement of the expression of the gene controlled by the endogenous promoter.

In still another embodiment, the knockout construct is inserted into the open reading frame region of a genomic locus such that the transcription initiation sequence in the knockout construct is oriented for transcription of a portion of the open reading frame of the gene, thereby activating expression or overexpressing the entire or a portion of sequence which encodes the protein. In one embodiment, the construct is inserted before the ATG codon, thereby activating production or overproduction of the entire encoded protein. In another embodiment, the construct is inserted after the ATG codon, thereby activating production or overproduction of a fragment of the encoded protein.

In one embodiment, a rapamycin resistance clone, e.g., a RHKO clone, is identified by treating the obtained clones with a suitable concentration of rapamycin for a suitable period of time. In a preferred embodiment, when an N2a cell line is used to generate the clone, a rapamycin resistance clone is identified by treating the obtained clones with 1 μM of rapamycin for 14 days. RHKO induced rapamycin resistance is further verified by selecting rapamycin resistant clones which exhibit reversible rapamycin resistance. As used herein, reversibility R is defined as a at least two fold reduction of rapamycin's inhibitory effect when the expression of the knockout construct is suppressed, e.g., when the expression of the transactivation factor is suppressed. For example, reversibility R may be defined as

$$R = \frac{\begin{matrix}\% Inhibition by Rapamycin when the \\ gene search construct is on\end{matrix}}{\begin{matrix}\% Inhibition by Rapamycin when the \\ gene search construct is off\end{matrix}} - 2 \qquad ( 1 )$$

In a preferred embodiment, the reversibility of rapamycin resistance is assayed by comparing rapamycin resistance in the presence and absence of the transactivation factor. RHKO clones that is rapamycin resistant in the presence of the transactivation factor and rapamycin sensitive in the absence of the transactivation factor are identified. In another preferred embodiment of the invention, a second construct comprising a marker gene and the transactivation factor that activate the transcription initiation sequence of the knockout construct operably linked to a regulated promoter is also introduced into the genome of the selected cell line. The activation of the knockout construct can then be regulated by activating or suppressing the regulated promoter in the second construct. In another preferred embodiment, the RHKO clones are further assayed using any standard method known in the art, e.g., Southern blotting, such that clones that contain a single copy of the knockout construct can be identified.

Once RHKO clones which exhibit reversible rapamycin resistance are identified, the genomic DNA sequence flanking the integration site of the knockout construct can be obtained and sequenced by any standard method known in the art. Preferably, the flanking genomic sequence obtained and sequenced is at least about 500 bases in length. More preferably, the flanking genomic sequence obtained and sequenced is at least about 1000 bases in length. Still more preferably, the flanking genomic sequence obtained and sequenced is between 500 to 5000 bases in length. Still more preferably, the flanking genomic sequence obtained and sequenced is in the range of 1000 to 3000 bases in length. In one embodiment, the entire open reading frame is obtained and sequenced. In another embodiment, the regulatory sequence is obtained and sequenced. In a preferred embodiment, more than one sequences for a clone may be obtained, and a consensus sequence is determined using any standard method known in the art. Most preferably, the regulatory sequences and the entire open reading frame are obtained and sequenced.

The obtained sequence can then be used as the query sequence to search one or more databases. Any method known in the art can be used for this purpose. The methods can make use of any sequence information available for the organism, including but not limited to, the genomic sequence data, the protein sequence data, mRNA sequence data, and EST data in conjunction with computational sequence analysis tools to identify the coding regions and or regulatory sequences in the genome of the organism. One skilled person in the art will be able to choose one or more methods, e.g., BLAST, and one or more appropriate databases, e.g., Ensembl, GenBank, etc. In a preferred embodiment, the structure of the gene, e.g., exon and/or intron sequences, is determined by comparing the expressed mRNA sequences or cDNAs or ESTs derived therefrom to the genomic sequence of the organism.

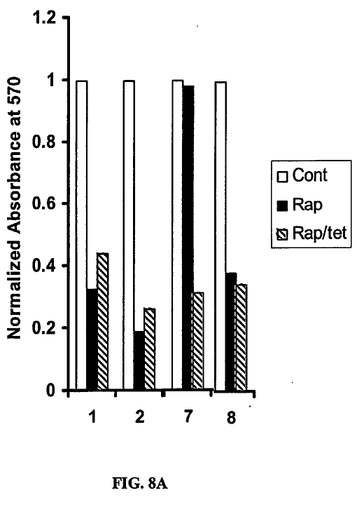

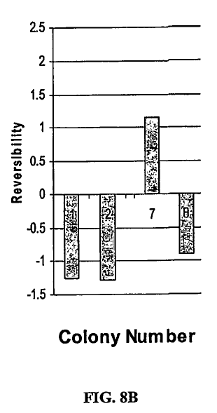

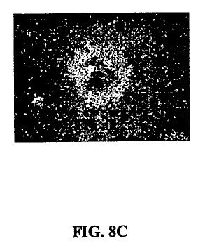

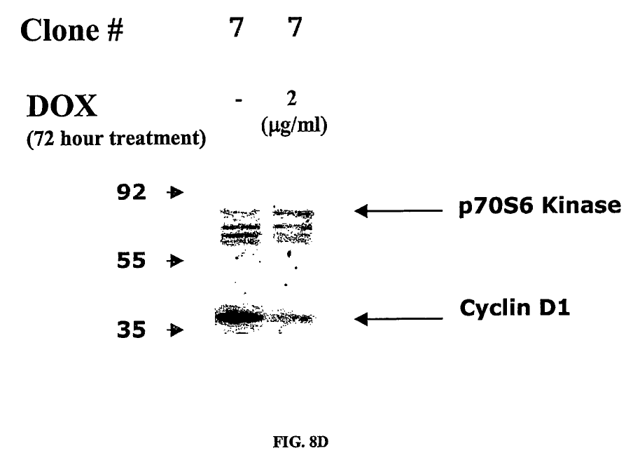

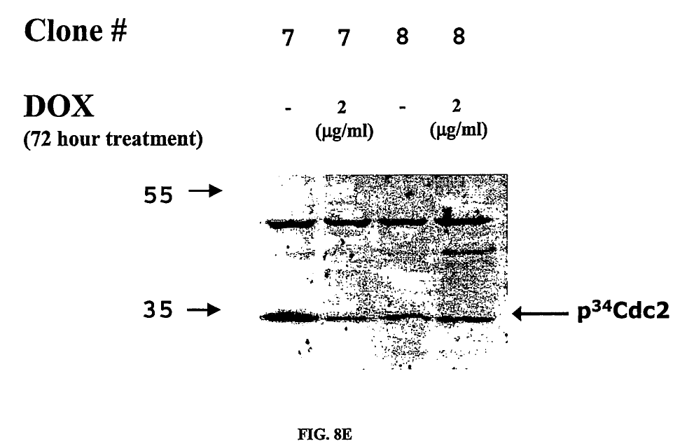

In one embodiment, the invention provides an RHKO clone, RapR7, which is a murine N2a cell containing a knockout construct inserted in the intron between exon 1 and exon 2 of a RapR7 gene and oriented for sense RNA transcription. Thus, when the expression of the knockout construct is activated, e.g., in the presence of a transactivator, the RapR7 gene is overexpressed in the RapR7 clone. FIGS. 8A-8E show that the RapR7 clone exhibits reversible rapamycin resistance and tumorgenesis. FIG. 1A shows a fragment of the genomic sequence flanking the insertion site.

### 5.2. RapR7 Gene Products and Cell Lines that Overexpress RapR7

The present invention provides RapR7 gene products, e.g., proteins or fragments thereof, cell lines that are engineered to express RapR7, as well as transgenic animals that are engineered to express RapR7.

### 5.2.1. RapR7 Gene Products

RapR7 gene products, or peptide fragments thereof, can be prepared for a variety of uses. For example, such gene products, or peptide fragments thereof, can be used for the generation of antibodies, in diagnostic assays, or for the identification of other cellular gene products involved in the regulation of expression and/or activities of RapR7 gene.

The amino acid sequences depicted in FIGS. 2B and 3B represents RapR7 gene products. The RapR7 gene product, sometimes referred to herein as an “RapR7 protein or polypeptide”, may additionally include those gene products encoded by the RapR7 gene sequences described in Section 5.1, above. In one embodiment, the invention provides a 951 amino acid murine RapR7 protein (PI: 5.6; MW: 105.47 kDa). In another embodiment, the invention provides an 850 amino acid human RapR7 protein (PI: 5.4; MW: 93.6 kDa).

In addition, RapR7 gene products may include proteins that represent functionally equivalent gene products. Such an equivalent RapR7 gene product may contain deletions, additions or substitutions of amino acid residues within the amino acid sequence encoded by the RapR7 gene sequences described, above, in Section 5.1, but which result in a silent change, thus producing a functionally equivalent RapR7 gene product. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

“Functionally equivalent”, as utilized herein, refers to a protein capable of exhibiting a substantially similar in vivo activity as the endogenous RapR7 gene products encoded by the RapR7 gene sequences described in Section 5.1, above. The in vivo activity of the RapR7 gene product, as used herein, refers to the ability of the RapR7 gene product, when present in an appropriate cell type, to ameliorate, prevent or delay the appearance of the RapR7 abnormal phenotype.

The invention also includes fragments of a RapR7 protein. In a preferred embodiment, the invention provides a fragment of a RapR7 protein comprising amino acids as described by SEQ ID NO:4 or 7. In one embodiment, the invention provides a fragment of a RapR7 protein comprising amino acids of a fragment comprising a PHD domain of a RapR7 protein. In a specific embodiment, the invention provides a polypeptide comprising amino acids 217-263 or 326-381 of a human RapR7 protein, or its murine homolog. In another embodiment, the invention provides a fragment of a RapR7 protein comprising amino acids of a fragment comprising a coiled-coil domain of a RapR7 protein. In a specific embodiment, the invention provides a polypeptide comprising amino acids 163-190 of the human RapR7 protein, or its murine homolog. In another embodiment, the invention provides a fragment of a RapR7 protein comprising amino acids of a fragment comprising a second peroximal domain of a RapR7 protein. In a specific embodiment, the invention provides a polypeptide comprising amino acids 514-522 of the human RapR7 protein, or its murine homolog. In another embodiment, the invention provides a fragment of a RapR7 protein comprising amino acids of a fragment comprising a nuclear localization domain of a RapR7 protein. In a specific embodiment, the invention provides a polypeptide comprising amino acids 20-23, 548-564, 549-565, 650-656, 661-667, or 663-666 of the human RapR7 protein, or its murine homolog. In another embodiment, the invention provides a fragment of a RapR7 protein comprising a low complexity domain of a RapR7 protein. In a specific embodiment, the invention provides a polypeptide comprising amino acids 9-28, 161-177 or 534-550 of a human RapR7 protein, or its murine homolog. In still another embodiment, the invention provides a polypeptide which comprises a fragment of a RapR7 protein but which does not comprises a PHD, a coiled-coil, a second peroximal domain, a nuclear localization domain, or a low complexity domain of a RapR7 protein. The invention also provides any sequence that is at least 30%, 50%, 70%, 90%, or 95% homologous such fragments of a RapR7 protein. The invention also provides peptides which are comprised in a RapR7 gene product and are at least 5, 10, 20, 50, 100 amino acids in length.

The RapR7 gene products or peptide fragments thereof, may be produced by recombinant DNA technology using techniques well known in the art. Thus, methods for preparing the RapR7 gene polypeptides and peptides of the invention by expressing nucleic acid containing RapR7 gene sequences are described herein. Methods which are well known to those skilled in the art can be used to construct expression vectors containing RapR7 gene product coding sequences and appropriate transcriptional and translational control signals. These methods include, for example, in vitro recombinant DNA techniques, synthetic techniques, and in vivo genetic recombination. See, for example, the techniques described in Sambrook et al., 1989, supra, and Ausubel et al., 1989, supra. Alternatively, RNA capable of encoding RapR7 gene product sequences may be chemically synthesized using, for example, synthesizers. See, for example, the techniques described in “Oligonucleotide Synthesis”, 1984, Gait, M. J. ed., IRL Press, Oxford, which is incorporated herein by reference in its entirety.

### 5.2.2. Cell Lines that Express RapR7

A variety of host-expression vector systems may be utilized to express the RapR7 gene coding sequences of the invention. Such host-expression systems represent vehicles by which the coding sequences of interest may be produced and subsequently purified, but also represent cells which may, when transformed or transfected with the appropriate nucleotide coding sequences, exhibit the RapR7 gene product of the invention in situ. These include but are not limited to microorganisms such as bacteria (e.g., E. coli, B. subtilis) transformed with recombinant bacteriophage DNA, plasmid DNA or cosmid DNA expression vectors containing RapR7 gene product coding sequences; yeast (e.g., Saccharomyces, Pichia) transformed with recombinant yeast expression vectors containing the RapR7 gene product coding sequences; insect cell systems infected with recombinant virus expression vectors (e.g., baculovirus) containing the RapR7 gene product coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing RapR7 gene product coding sequences; or mammalian cell systems (e.g., COS, CHO, BHK, 293, 3T3, N2a) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the RapR7 gene product being expressed. For example, when a large quantity of such a protein is to be produced, for the generation of pharmaceutical compositions of RapR7 protein or for raising antibodies to RapR7 protein, for example, vectors which direct the expression of high levels of fusion protein products that are readily purified may be desirable. Such vectors include, but are not limited, to the E. coli expression vector pUR278 (Ruther et al., 1983, EMBO J. 2:1791), in which the RapR7 gene product coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 264:5503-5509); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The RapR7 gene coding sequence may be cloned individually into non-essential regions (for example the polyhedrin gene) of the virus and placed under control of an AcNPV promoter (for example the polyhedrin promoter). Successful insertion of RapR7 gene coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed. (E.g., see Smith et al., 1983, J. Virol. 46: 584; Smith, U.S. Pat. No. 4,215,051).

In mammalian host cells, a number of viral-based expression systems may be utilized. In cases where an adenovirus is used as an expression vector, the RapR7 gene coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing RapR7 gene product in infected hosts. (E.g., See Logan & Shenk, 1984, Proc. Natl. Acad. Sci. USA 81:3655-3659). Specific initiation signals may also be required for efficient translation of inserted RapR7 gene product coding sequences. These signals include the ATG initiation codon and adjacent sequences. In cases where an entire RapR7 gene, including its own initiation codon and adjacent sequences, is inserted into the appropriate expression vector, no additional translational control signals may be needed. However, in cases where only a portion of the RapR7 gene coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:516-544). In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, WI38.

For long-term, high-yield production of recombinant proteins, stable expression is preferred. For example, cell lines which stably express the RapR7 gene product may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the RapR7 gene product. Such engineered cell lines may be particularly useful in screening and evaluation of compounds that affect the endogenous activity of the RapR7 gene product.

In another embodiment, the expression characteristics of an endogenous gene (e.g., a RapR7 gene) within a cell, cell line or microorganism may be modified by inserting a DNA regulatory element heterologous to the endogenous gene of interest into the genome of a cell, stable cell line or cloned microorganism such that the inserted regulatory element is operatively linked with the endogenous gene (e.g., a RapR7 gene) and controls, modulates, activates, or inhibits the endogenous gene. For example, endogenous RapR7 genes which are normally “transcriptionally silent”, i.e., a RapR7 gene which is normally not expressed, or is expressed only at very low levels in a cell line or microorganism, may be activated by inserting a regulatory element which is capable of promoting the expression of the gene product in that cell line or microorganism. Alternatively, transcriptionally silent, endogenous RapR7 genes may be activated by insertion of a promiscuous regulatory element that works across cell types.

A heterologous regulatory element may be inserted into a stable cell line or cloned microorganism, such that it is operatively linked with and activates or inhibits expression of endogenous RapR7 genes, using techniques, such as targeted homologous recombination, which are well known to those of skill in the art, and described e.g., in Chappel, U.S. Pat. No. 5,272,071; PCT Publication No. WO 91/06667 published May 16, 1991; Skoultchi, U.S. Pat. No. 5,981,214; and Treco et al U.S. Pat. No. 5,968,502 and PCT Publication No. WO 94/12650 published Jun. 9, 1994. Alternatively, non-targeted, e.g. non-homologous recombination techniques may be used which are well-known to those of skill in the art and described, e.g., in PCT Publication No. WO 99/15650 published Apr. 1, 1999.

RapR7 gene activation (or inactivation) may also be accomplished using designer transcription factors using techniques well known in the art. Briefly, a designer zinc finger protein transcription factor (ZFP-TF) is made which is specific for a regulatory region of the RapR7 gene to be activated or inactivated. A construct encoding this designer ZFP-TF is then provided to a host cell in which the RapR7 gene is to be controlled. The construct directs the expression of the designer ZFP-TF protein, which in turn specifically modulates the expression of the endogenous RapR7 gene. The following references relate to various aspects of this approach in further detail: Wang & Pabo, 1999, Proc. Natl. Acad. Sci. USA 96, 9568; Berg, 1997, Nature Biotechnol. 15, 323; Greisman & Pabo, 1997, Science 275, 657; Berg & Shi, 1996, Science 271, 1081; Rebar & Pabo, 1994, Science 263, 671; Rhodes & Klug, 1993, Scientific American 269, 56; Pavletich & Pabo, 1991, Science 252, 809; Liu et al., 2001, J. Biol. Chem. 276, 11323; Zhang et al., 2000, J. Biol. Chem. 275, 33850; Beerli et al., 2000, Proc. Natl. Acad. Sci. USA 97, 1495; Kang et al., 2000, J. Biol. Chem. 275, 8742; Beerli et al., 1998, Proc. Natl. Acad. Sci. USA 95, 14628; Kim & Pabo, 1998, Proc. Natl. Acad. Sci. USA 95, 2812; Choo et al., 1997, J. Mol. Biol. 273, 525; Kim & Pabo, 1997, J. Biol. Chem. 272, 29795; Liu et al, 1997, Proc. Natl. Acad. Sci. USA 94, 5525; Kim et al, 1997, Proc. Natl. Acad. Sci. USA 94, 3616; Kikyo et al., 2000, Science 289, 2360; Robertson & Wolffe, 2000, Nature Reviews 1, 11; and Gregory, 2001, Curr. Opin. Genet. Devt. 11, 142.

A number of selection systems may be used, including but not limited to the herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy, et al., 1980, Cell 22:817) genes can be employed in tk⁻, hgprt⁻ or aprt⁻ cells, respectively. Also, antimetabolite resistance can be used as the basis of selection for the following genes: dhfr, which confers resistance to methotrexate (Wigler, et al., 1980, Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150:1); and hygro, which confers resistance to hygromycin (Santerre, et al., 1984, Gene 30:147).

Alternatively, any fusion protein may be readily purified by utilizing an antibody specific for the fusion protein being expressed. For example, a system described by Janknecht et al. allows for the ready purification of non-denatured fusion proteins expressed in human cell lines (Janknecht, et al., 1991, Proc. Natl. Acad. Sci. USA 88: 8972-8976). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺-nitriloacetic acid-agarose columns and histidine-tagged proteins are selectively eluted with imidazole-containing buffers.

### 5.2.3. Cell Lines that Overexpress RapR7 Reversibly

The invention also provides cell lines in which overexpression of RapR7 gene can be switched on or off, as produced by the method described in Section 5.1.2. Such cell lines are useful, e.g., for identifying RapR7 related cellular pathways and/or for screening for agents that modulate the expression of RapR7 gene and/or the interactions of RapR7 gene with other molecules.

### 5.2.4. Transgenic Animals that Express RapR7

The RapR7 gene products can also be expressed in transgenic animals. Animals of any species, including, but not limited to, mice, rats, rabbits, guinea pigs, pigs, micro-pigs, goats, and non-human primates, e.g., baboons, monkeys, and chimpanzees may be used to generate RapR7 transgenic animals.

Any technique known in the art may be used to introduce the RapR7 gene transgene into animals to produce the founder lines of transgenic animals. Such techniques include, but are not limited to pronuclear microinjection (Hoppe, P. C. and Wagner, T. E., 1989, U.S. Pat. No. 4,873,191); retrovirus mediated gene transfer into germ lines (Van der Putten et al, 1985, Proc. Natl. Acad. Sci., USA 82:6148-6152); gene targeting in embryonic stem cells (Thompson et al., 1989, Cell 56:313-321); electroporation of embryos (Lo, 1983, Mol Cell. Biol. 3:1803-1814); and sperm-mediated gene transfer (Lavitrano et al., 1989, Cell 57:717-723); etc. For a review of such techniques, see Gordon, 1989, Transgenic Animals, Intl. Rev. Cytol. 115:171-229, which is incorporated by reference herein in its entirety.

The present invention provides for transgenic animals that carry the RapR7 transgene in all their cells, as well as animals which carry the transgene in some, but not all their cells, i.e., mosaic animals. The transgene may be integrated as a single transgene or in concatamers, e.g., head-to-head tandems or head-to-tail tandems. The transgene may also be selectively introduced into and activated in a particular cell type by following, for example, the teaching of Lasko et al. (Lasko, M. et al., 1992, Proc. Natl. Acad. Sci. USA 89: 6232-6236). The regulatory sequences required for such a cell-type specific activation will depend upon the particular cell type of interest, and will be apparent to those of skill in the art. When it is desired that the RapR7 transgene be integrated into the chromosomal site of the endogenous RapR7 gene, gene targeting is preferred. Briefly, when such a technique is to be utilized, vectors containing some nucleotide sequences homologous to the endogenous RapR7 gene are designed for the purpose of integrating, via homologous recombination with chromosomal sequences, into and disrupting the function of the nucleotide sequence of the endogenous RapR7 gene. The transgene may also be selectively introduced into a particular cell type, thus inactivating the endogenous RapR7 gene in only that cell type, by following, for example, the teaching of Gu et al. (Gu, et al., 1994, Science 265: 103-106). The regulatory sequences required for such a cell-type specific inactivation will depend upon the particular cell type of interest, and will be apparent to those of skill in the art.

Once transgenic animals have been generated, the expression of the recombinant RapR7 gene may be assayed utilizing standard techniques. Initial screening may be accomplished by Southern blot analysis or PCR techniques to analyze animal tissues to assay whether integration of the transgene has taken place. The level of mRNA expression of the transgene in the tissues of the transgenic animals may also be assessed using techniques which include but are not limited to Northern blot analysis of tissue samples obtained from the animal, in situ hybridization analysis, and RT-PCR. Samples of RapR7 gene-expressing tissue, may also be evaluated immunocytochemically using antibodies specific for the RapR7 transgene product.

### 5.3. Antibodies to RapR7 Gene Products

The present invention provides antibodies that bind to RapR7 gene products, e.g., a human RapR7 protein or a fragment thereof. Such antibodies may include, but are not limited to, polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above. Such antibodies may be used, for example, in the detection of a RapR7 gene product in an biological sample and may, therefore, be utilized as part of a diagnostic or prognostic technique whereby patients may be tested for abnormal levels of RapR7 gene products, and/or for the presence of abnormal forms of the such gene products. Such antibodies may also be utilized in conjunction with, for example, compound screening schemes, as described, below, in Section 5.4.2, for the evaluation of the effect of test compounds on RapR7 gene product levels and/or activity. Additionally, such antibodies can be used in conjunction with the gene therapy techniques described, below, in Section 5.4.3, to, for example, evaluate the normal and/or engineered RapR7-expressing cells prior to their introduction into the patient.

Anti-RapR7 gene product antibodies may also be used for the inhibition of abnormal RapR7 gene product activity. Anti-RapR7 gene product antibodies may additionally be used for the inhibition of abnormal RapR7 gene product activity. Thus, such antibodies may, therefore, be utilized as part of treatment methods of a disease resulting from defective regulation of RapR7 gene expression and/or abnormal RapR7 gene product activity.

### 5.3.1. Methods of Screening for Antibodies Directed to RapR7 Protein and Domains/Fragments of RapR7 Protein

The present invention provides methods for screening for antibodies that bind to RapR7 protein. The methods involve screening for antibodies using an appropriate polypeptides of a RapR7 protein. Any fragment of the RapR7 protein, e.g., those described in Section 5.2., can be used to raise the antibody of the invention.

Screening for desired antibody can be accomplished by techniques known in the art. In one embodiment, antibodies which recognize a specific domain of a RapR7, generated hybridomas are assayed for a product which binds to a RapR7 fragment containing such domain.

In another embodiment, an antibody directed against RapR7 protein or a fragment/polypeptide of a RapR7 protein can be identified and isolated by screening a recombinant combinatorial immunoglobulin library (e.g., an antibody phage display library) with the RapR7 protein or a fragment/polypeptide of a RapR7 protein. Kits for generating and screening phage display libraries are commercially available (e.g. Pharmacia Recombinant Phage Antibody System, Catalog No. 27-9400-01; and the Stratagene antigen SurfZAP™ Phage Display Kit, Catalog No. 240612). Additionally, examples of methods and reagents particularly amenable for use in generating and screening antibody display library can be found in, for example, U.S. Pat. Nos. 5,223,409 and 5,514,548; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734. A phage display library permits selection of desired antibody or antibodies from a very large repertoire of specificities. An additional advantage of a phage display library is that the nucleic acids encoding the selected antibodies can be obtained conveniently, thereby facilitating subsequent construction of expression vectors.

For selection of an antibody that specifically binds a particular isoform of RapR7 but which does not specifically bind other isoforms of RapR7, by any of the above mentioned methods of this section, one can select on the basis of positive binding to the desired isoform of RapR7 and a lack of binding to the other isoforms. In a preferred embodiment, the sequence of a RapR7 fragment used for the selection of antibodies is a sequence not comprised by other isoforms of RapR7 whose activities are to be preserved.

In one embodiment, the invention provides an antibody that binds the 951 amino acid murine RapR7 protein (SEQ ID NO:3). In another embodiment, the invention provides an antibody that binds the 850 amino acid human RapR7 protein (SEQ ID NO:6). In one embodiment, the invention provides an antibody that binds a fragment of a RapR7 protein comprising amino acids as described by SEQ ID NO:4 or 7. In another embodiment, the invention provides an antibody that binds a polypeptide comprising a PHD domain, a coiled-coil domain, a second peroximal domain, a nuclear localization domain, or a low complexity domain of a human or murine RapR7 protein. In specific embodiments, the invention provides an antibody that binds a polypeptide comprising amino acids 217-263, 326-381, 163-190, 514-522, 20-23, 548-564, 549-565, 650-656, 661-667, 663-666, 9-28, 161-177, or 534-550 of a human RapR7 protein, or a polypeptide comprising the corresponding murine homologs. In still another embodiment, the invention provides an antibody that binds a polypeptide which comprises a fragment of a RapR7 protein but which does not comprises a PHD, a coiled-coil, a second peroximal domain, a nuclear localization domain, or a low complexity domain of a RapR7 protein. The invention also provides an antibody that binds any sequence that is at least 30%, 50%, 70%, 90%, or 95% homologous to such fragments of a RapR7 protein.

### 5.3.2. Methods of Production of Antibodies

Described herein are methods for the production of antibodies capable of specifically recognizing a sequence of a RapR7 gene or one or more RapR7 gene product epitopes or epitopes of conserved variants or peptide fragments of the RapR7 gene products.

For the production of antibodies against a RapR7 gene product, various host animals may be immunized by injection with a RapR7 gene product, or a portion thereof. Such host animals may include but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as a RapR7 gene product, or an antigenic functional derivative thereof. For the production of polyclonal antibodies, host animals such as those described above, may be immunized by injection with RapR7 gene product supplemented with adjuvants as also described above.

Monoclonal antibodies, which are homogeneous populations of antibodies to a particular antigen, such as a RapR7 gene product, or an antigenic functional derivative thereof, may be obtained by any technique which provides for the production of antibody molecules by continuous cell lines in culture. These include, but are not limited to, the hybridoma technique of Kohler and Milstein, (1975, Nature 256:495-497; and U.S. Pat. No. 4,376,110), the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison et al., 1984, Proc. Natl. Acad. Sci., 81:6851-6855; Neuberger et al., 1984, Nature, 312:604-608; Takeda et al., 1985, Nature, 314:452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region.

Alternatively, techniques described for the production of single chain antibodies (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546) can be adapted to produce single chain antibodies against RapR7 gene products. Single chain antibodies are formed by linking the heavy and light chain fragments of the Fv region via an amino acid bridge, resulting in a single chain polypeptide.

Antibody fragments which recognize specific epitopes may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., 1989, Science, 246:1275-1281) to allow rapid and easy identification of monoclonal Fab fragments with the desired specificity.

### 5.4. Uses of RapR7 Gene, Gene Products, Cell Lines and Antibodies

The invention provides methods and compositions for utilizing the RapR7 gene, product and antibodies for identifying proteins or other molecules that interact with RapR7 gene or protein. The invention also provides methods and compositions for utilizing the RapR7 gene, product and antibodies for screening for agents that RapR7 expression or modulating interaction of RapR7 gene or protein with other proteins or molecules. The invention further provides methods and compositions for utilizing the RapR7 gene, product and antibodies for screening for agents that are useful in regulating rapamycin resistance and/or tumorgenesis in a cell or organism. The invention also provides methods and compositions for utilizing RapR7 gene, product and antibodies for diagnosing RapR7 mediated rapamycin resistance and/or tumorgenesis, and for treatment of diseases in conjunction with a rapamycin therapy.

### 5.4.1. Methods of Determining Proteins or Other Molecules that Interact with RapR7 Gene or Gene Product

Any method suitable for detecting protein-protein interactions may be employed for identifying RapR7 protein-cellular protein interactions. The interaction between RapR7 gene and other cellular molecules, e.g., interaction between RapR7 and its regulators may also be determined using methods known in the art.

Among the traditional methods which may be employed are co-immunoprecipitation, crosslinking and co-purification through gradients or chromatographic columns. Utilizing procedures such as these allows for the identification of cellular proteins which interact with RapR7 gene products. Once isolated, such an cellular protein can be identified and can, in turn, be used, in conjunction with standard techniques, to identify proteins it interacts with. For example, at least a portion of the amino acid sequence of the cellular protein which interacts with the RapR7 gene product can be ascertained using techniques well known to those of skill in the art, such as via the Edman degradation technique (see, e.g., Creighton, 1983, “Proteins: Structures and Molecular Principles”, W.H. Freeman & Co., N.Y., pp. 34-49). The amino acid sequence obtained may be used as a guide for the generation of oligonucleotide mixtures that can be used to screen for gene sequences encoding such cellular proteins. Screening made be accomplished, for example, by standard hybridization or PCR techniques. Techniques for the generation of oligonucleotide mixtures and the screening are well-known. (See, e.g., Ausubel, supra., and PCR Protocols: A Guide to Methods and Applications, 1990, Innis, M. et al., eds. Academic Press, Inc., New York).

Additionally, methods may be employed which result in the simultaneous identification of genes which encode the cellular protein interacting with the RapR7 protein. These methods include, for example, probing expression libraries with labeled RapR7 protein, using RapR7 protein in a manner similar to the well known technique of antibody probing of λgt11 libraries.

One method which detects protein interactions in vivo, the two-hybrid system, is described in detail for illustration only and not by way of limitation. One version of this system has been described (Chien et al., 1991, Proc. Natl. Acad. Sci. USA, 88:9578-9582) and is commercially available from Clontech (Palo Alto, Calif.).

Briefly, utilizing such a system, plasmids are constructed that encode two hybrid proteins: one consists of the DNA-binding domain of a transcription activator protein fused to the RapR7 gene product and the other consists of the transcription activator protein's activation domain fused to an unknown protein that is encoded by a cDNA which has been recombined into this plasmid as part of a cDNA library. The DNA-binding domain fusion plasmid and the cDNA library are transformed into a strain of the yeast Saccharomyces cerevisiae that contains a reporter gene (e.g., HBS or lacz) whose regulatory region contains the transcription activator's binding site. Either hybrid protein alone cannot activate transcription of the reporter gene: the DNA-binding domain hybrid cannot because it does not provide activation function and the activation domain hybrid cannot because it cannot localize to the activator's binding sites. Interaction of the two hybrid proteins reconstitutes the functional activator protein and results in expression of the reporter gene, which is detected by an assay for the reporter gene product.

The two-hybrid system or related methodology may be used to screen activation domain libraries for proteins that interact with the “bait” gene product. By way of example, and not by way of limitation, RapR7 gene products may be used as the bait gene product. Total genomic or cDNA sequences are fused to the DNA encoding an activation domain. This library and a plasmid encoding a hybrid of a bait RapR7 gene product fused to the DNA-binding domain are cotransformed into a yeast reporter strain, and the resulting transformants are screened for those that express the reporter gene. For example, and not by way of limitation, a bait RapR7 gene sequence, such as the coding sequence of a RapR7 gene can be cloned into a vector such that it is translationally fused to the DNA encoding the DNA-binding domain of the GALA protein. These colonies are purified and the library plasmids responsible for reporter gene expression are isolated. DNA sequencing is then used to identify the proteins encoded by the library plasmids.

A cDNA library of the cell line from which proteins that interact with bait RapR7 gene product are to be detected can be made using methods routinely practiced in the art. According to the particular system described herein, for example, the cDNA fragments can be inserted into a vector such that they are translationally fused to the transcriptional activation domain of GAL4. This library can be co-transformed along with the bait RapR7 gene-GAL4 fusion plasmid into a yeast strain which contains a lacZ gene driven by a promoter which contains GAL4 activation sequence. A cDNA encoded protein, fused to GALA transcriptional activation domain, that interacts with bait RapR7 gene product will reconstitute an active GAL4 protein and thereby drive expression of the HIS3 gene. Colonies which express HIS3 can be detected by their growth on petri dishes containing semi-solid agar based media lacking histidine. The cDNA can then be purified from these strains, and used to produce and isolate the bait RapR7 gene-interacting protein using techniques routinely practiced in the art.

The interaction between a RapR7 gene and its regulators may be determined by a standard method known in the art.

In one embodiment, the invention provides Cyclin D1 and cdc2 as a molecule involved in the RapR7 related rapamyin regulatory pathway (see FIGS. 8D-8E). Elevated expression of cyclin D1 and/or cdc2 have been shown in various cancers (see, e.g., Shintani et al., 2002, Oral Oncol. 38:235-43). Cdc2 protein kinase (also termed p34 protein kinase) which is activated by forming a complex with cyclin B and is required for the G2/M transition, i.e., the transition from G2 phase to mitosis, of the cell cycle (see, e.g., Smits et al., 2001, Biochim Biophys Acta 1519:1-12; Draetta et al., 1988, Cell 54:17-26; Lee et al., 1987, Nature 327:31-35; Pines et al., 1989, Cell 58:833-846). Reduction in cdc2 expression and/or activity has been shown to lead to G2/M arrest. An analysis of the murine cdc2 gene has shown that cdc2 gene, while lacking a TATA box in its promoter region, utilizes multiple transcriptional start sites, including transcription factor binding sites for PEA3, CREB, C/EBP, E box factor, YY1, ATF-like, Spl, and E2F (Jun et al., 1998, Mol. Cells 8:731-40). Therefore the expression of cdc2 gene may be controlled by a variety of different factors. For example, it has been reported that a checkpoint protein Chfr delays entry into mitosis via negatively regulating phosphorylation of cdc2 (Kang et al., 2002, J. Cell Biol. 156:249-60). It has also been reported that LATS1, a mammalian tumor suppressor gene, inhibits cell proliferation by reducing cdc2 kinase activity and causing G2/M blockade (Xia et al., 2002, Oncogene 21:1233-41). p53 has also been shown to negatively regulate cdc2 gene expression via binding to an inverted CCAAT sequence in the presence of the transcription factor NF-Y (Yun et al., 1999, J. Biol. Chem. 274:29677-82). Other cellular proteins that regulate cdc2 gene expression include the upstream stimulatory factors, a subset of Helix-Loop-Helix family of transcription factors, which binds to the CAGGTGGC sequence contained in an E-box (North et al., 1999, Oncogen 18:1945-55). Extracellular factors may also affect cdc2 expression and/or activity and lead to G2/M arrest. For example, it has been reported that the inhibition of phosphorylation of cdc2 by a reovirus leads to inhibition of cellular proliferation by inducing G2/M cell cycle arrect (Poggioli et al., 2001, J Virol 75(16):7429-34). It has also been reported that the anti-cancer activity of a synthetic quinoxaline phenoxypropionic acid derivative, 2-[4-(7-chloro-2-quinoxalinyloxy)phenoxy]propionic acid, is achieved by inducing G2/M arrest via inactivation of cdc2 kinase activity (Ding et al., 2001, Clin Cancer Res 7:3336-42).

Cyclin D1 is one of G1 cyclins. Suppression of cyclin D1 was shown to inhibit cell cycle at G0/G1. It is also reported that the all-trans-retinoic acid triggered G1 arrest is at least partly through proteasome-dependent degradation of cyclin D1 (Dragnev et al., 2001, Annals of the New York Academy of Sciences 952:13-22).

### 5.4.2. Methods of Screening for Agents

The invention provides methods for screening for agents that regulate RapR7 expression or modulate interaction of RapR7 with other proteins or molecules.

### 5.4.2.1. Screening Assays

The following assays are designed to identify compounds that bind to RapR7 gene or gene products, bind to other cellular proteins that interact with a RapR7 gene product, bind to cellular constituents, e.g., proteins, that are affected by a RapR7 gene product, or bind to compounds that interfere with the interaction of the RapR7 gene or gene product with other cellular proteins and to compounds which modulate the activity of RapR7 gene (i.e., modulate the level of RapR7 gene expression and/or modulate the level of RapR7 gene product activity). Assays may additionally be utilized which identify compounds which bind to RapR7 gene regulatory sequences (e.g., promoter sequences), see e.g., Platt, K. A., 1994, J. Biol. Chem. 269:28558-28562, which is incorporated herein by reference in its entirety, which may modulate the level of RapR7 gene expression. Compounds may include, but are not limited to, small organic molecules which are able to affect expression of the RapR7 gene or some other gene involved in the rapamycin resistance regulatory pathways, or other cellular proteins. For example, the invention provides Cyclin D1 and cdc2 as molecules involved in the RapR7 related rapamyin regulatory pathway (see FIGS. 8D-8E). Methods for the identification of such cellular proteins are described, above, in Section 5.4.1. Such cellular proteins may be involved in the control and/or regulation of rapamycin resistance and/or tumorgenesis. Further, among these compounds are compounds which affect the level of RapR7 gene expression and/or RapR7 gene product activity and which can be used in the regulation of rapamycin resistance and/or tumorgenesis.

Compounds may include, but are not limited to, peptides such as, for example, soluble peptides, including but not limited to, Ig-tailed fusion peptides, and members of random peptide libraries; (see, e.g., Lam, K. S. et al., 1991, Nature 354:82-84; Houghten, R. et al., 1991, Nature 354:84-86), and combinatorial chemistry-derived molecular library made of D- and/or L-configuration amino acids, phosphopeptides (including, but not limited to members of random or partially degenerate, directed phosphopeptide libraries; see, e.g., Songyang, Z. et al., 1993, Cell 72:767-778), antibodies (including, but not limited to, polyclonal, monoclonal, humanized, anti-idiotypic, chimeric or single chain antibodies, and FAb, F(ab′)₂ and FAb expression library fragments, and epitope-binding fragments thereof), and small organic or inorganic molecules.

Compounds identified via assays such as those described herein may be useful, for example, in regulating the biological function of the RapR7 gene product, and for ameliorating rapamycin resistance and/or inhibiting the growth of cancer cells. Assays for testing the effectiveness of compounds are discussed, below, in Section 5.4.2.2.

In vitro systems may be designed to identify compounds capable of binding the RapR7 gene products of the invention. Compounds identified may be useful, for example, in modulating the activity of wild type and/or mutant RapR7 gene products, may be useful in elaborating the biological function of the RapR7 gene product, may be utilized in screens for identifying compounds that disrupt normal RapR7 gene product interactions, or may in themselves disrupt such interactions.

The principle of the assays used to identify compounds that bind to the RapR7 gene product involves preparing a reaction mixture of the RapR7 gene product and the test compound under conditions and for a time sufficient to allow the two components to interact and bind, thus forming a complex which can be removed and/or detected in the reaction mixture. These assays can be conducted in a variety of ways. For example, one method to conduct such an assay would involve anchoring RapR7 gene product or the test substance onto a solid phase and detecting RapR7 gene product/test compound complexes anchored on the solid phase at the end of the reaction. In one embodiment of such a method, the RapR7 gene product may be anchored onto a solid surface, and the test compound, which is not anchored, may be labeled, either directly or indirectly.

In practice, microtiter plates may conveniently be utilized as the solid phase. The anchored component may be immobilized by non-covalent or covalent attachments. Non-covalent attachment may be accomplished by simply coating the solid surface with a solution of the protein and drying. Alternatively, an immobilized antibody, preferably a monoclonal antibody, specific for the protein to be immobilized may be used to anchor the protein to the solid surface. The surfaces may be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the previously nonimmobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for RapR7 gene product or the test compound to anchor any complexes formed in solution, and a labeled antibody specific for the other component of the possible complex to detect anchored complexes.

The RapR7 gene or gene products of the invention may, in vivo, interact with one or more intracellular or extracellular molecules, such as proteins. Such molecules may include, but are not limited to, nucleic acid molecules and those proteins identified via methods such as those described, above, in Section 5.4.1. For purposes of this discussion, such molecules are referred to herein as “binding partners”. Compounds that disrupt RapR7 gene product binding may be useful in regulating the activity of the RapR7 gene product, especially mutant RapR7 gene products. Compounds that disrupt RapR7 gene binding may be useful in regulating the expression of the RapR7 gene, such as by regulating the binding of a regulator of RapR7 gene. Such compounds may include, but are not limited to molecules such as peptides, and the like, as described, for example, in Section 5.4.2.1. above, which would be capable of gaining access to the RapR7 gene product.

The basic principle of the assay systems used to identify compounds that interfere with the interaction between the RapR7 gene product and its intracellular or extracellular binding partner or partners involves preparing a reaction mixture containing the RapR7 gene product, and the binding partner under conditions and for a time sufficient to allow the two to interact and bind, thus forming a complex. In order to test a compound for inhibitory activity, the reaction mixture is prepared in the presence and absence of the test compound. The test compound may be initially included in the reaction mixture, or may be added at a time subsequent to the addition of RapR7 gene product and its binding partner. Control reaction mixtures are incubated without the test compound or with a placebo. The formation of any complexes between the RapR7 gene protein and the binding partner is then detected. The formation of a complex in the control reaction, but not in the reaction mixture containing the test compound, indicates that the compound interferes with the interaction of the RapR7 gene protein and the interactive binding partner. Additionally, complex formation within reaction mixtures containing the test compound and normal RapR7 gene protein may also be compared to complex formation within reaction mixtures containing the test compound and a mutant RapR7 gene protein. This comparison may be important in those cases wherein it is desirable to identify compounds that disrupt interactions of mutant but not normal RapR7 gene proteins.

The assay for compounds that interfere with the interaction of the RapR7 gene products and binding partners can be conducted in a heterogeneous or homogeneous format. Heterogeneous assays involve anchoring either the RapR7 gene product or the binding partner onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the RapR7 gene products and the binding partners, e.g., by competition, can be identified by conducting the reaction in the presence of the test substance; i.e., by adding the test substance to the reaction mixture prior to or simultaneously with the RapR7 gene protein and interactive binding partner. Alternatively, test compounds that disrupt preformed complexes, e.g. compounds with higher binding constants that displace one of the components from the complex, can be tested by adding the test compound to the reaction mixture after complexes have been formed. The various formats are described briefly below.

In a heterogeneous assay system, either the RapR7 gene product or the interactive binding partner, is anchored onto a solid surface, while the non-anchored species is labeled, either directly or indirectly. In practice, microtiter plates are conveniently utilized. The anchored species may be immobilized by non-covalent or covalent attachments. Non-covalent attachment may be accomplished simply by coating the solid surface with a solution of the RapR7 gene product or binding partner and drying. Alternatively, an immobilized antibody specific for the species to be anchored may be used to anchor the species to the solid surface. The surfaces may be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, may be directly labeled or indirectly labeled with a labeled anti-Ig antibody). Depending upon the order of addition of reaction components, test compounds which inhibit complex formation or which disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds which inhibit complex or which disrupt preformed complexes can be identified.

In an alternate embodiment of the invention, a homogeneous assay can be used. In this approach, a preformed complex of the RapR7 gene protein and the interactive binding partner is prepared in which either the RapR7 gene product or its binding partners is labeled, but the signal generated by the label is quenched due to complex formation (see, e.g., U.S. Pat. No. 4,109,496 by Rubenstein which utilizes this approach for immunoassays). The addition of a test substance that competes with and displaces one of the species from the preformed complex will result in the generation of a signal above background. In this way, test substances which disrupt RapR7 gene protein/binding partner interaction can be identified.

In a particular embodiment, the RapR7 gene product can be prepared for immobilization using recombinant DNA techniques described in Section 5.2. above. For example, the RapR7 coding region can be fused to a glutathione-S-transferase (GST) gene using a fusion vector, such as pGEX-5X-1, in such a manner that its binding activity is maintained in the resulting fusion protein. The interactive binding partner can be purified and used to raise a monoclonal antibody, using methods routinely practiced in the art and described above, in Section 5.3. This antibody can be labeled with the radioactive isotope ¹²⁵I, for example, by methods routinely practiced in the art. In a heterogeneous assay, e.g., the GST-RapR7 fusion protein can be anchored to glutathione-agarose beads. The interactive binding partner can then be added in the presence or absence of the test compound in a manner that allows interaction and binding to occur. At the end of the reaction period, unbound material can be washed away, and the labeled monoclonal antibody can be added to the system and allowed to bind to the complexed components. The interaction between the RapR7 gene protein and the interactive binding partner can be detected by measuring the amount of radioactivity that remains associated with the glutathione-agarose beads. A successful inhibition of the interaction by the test compound will result in a decrease in measured radioactivity.

Alternatively, the GST-RapR7 gene fusion protein and the interactive binding partner can be mixed together in liquid in the absence of the solid glutathione-agarose beads. The test compound can be added either during or after the species are allowed to interact. This mixture can then be added to the glutathione-agarose beads and unbound material is washed away. Again the extent of inhibition of the RapR7 gene product/binding partner interaction can be detected by adding the labeled antibody and measuring the radioactivity associated with the beads.

In another embodiment of the invention, these same techniques can be employed using peptide fragments that correspond to the binding domains of the RapR7 protein and/or the interactive binding partner (in cases where the binding partner is a protein), in place of one or both of the full length proteins. Any number of methods routinely practiced in the art can be used to identify and isolate the binding sites. These methods include, but are not limited to, mutagenesis of the gene encoding one of the proteins and screening for disruption of binding in a co-immunoprecipitation assay. Compensating mutations in the gene encoding the second species in the complex can then be selected. Sequence analysis of the genes encoding the respective proteins will reveal the mutations that correspond to the region of the protein involved in interactive binding. Alternatively, one protein can be anchored to a solid surface using methods described in this Section above, and allowed to interact with and bind to its labeled binding partner, which has been treated with a proteolytic enzyme, such as trypsin. After washing, a short, labeled peptide comprising the binding domain may remain associated with the solid material, which can be isolated and identified by amino acid sequencing. Also, once the gene coding for the binding partner is obtained, short gene segments can be engineered to express peptide fragments of the protein, which can then be tested for binding activity and purified or synthesized.

For example, and not by way of limitation, a RapR7 gene product can be anchored to a solid material as described, above, in this Section by making a GST-RapR7 fusion protein and allowing it to bind to glutathione agarose beads. The interactive binding partner can be labeled with a radioactive isotope, such as ³⁵S, and cleaved with a proteolytic enzyme such as trypsin. Cleavage products can then be added to the anchored GST-RapR7 fusion protein and allowed to bind. After washing away unbound peptides, labeled bound material, representing the binding partner binding domain, can be eluted, purified, and analyzed for amino acid sequence by well-known methods. Peptides so identified can be produced synthetically or fused to appropriate facilitative proteins using recombinant DNA technology.

### 5.4.2.2. Screening Compounds that Regulate Rapamycin Resistance and/or Tumorgenesis

Any agents that regulate the expression of RapR7 gene and/or the interaction of RapR7 protein with its binding partners, e.g., compounds that are identified in Section 5.4.2.1., antibodies to RapR7 protein, and so on, can be further screened for its ability to regulate rapamycin resistance and/or tumorgenesis in cells. Any suitable proliferation or growth inhibition assays known in the art can be used for this purpose. In one embodiment, a candidate agent and rapamycin are applied to a cells of a cell line, such as but not limited to, a rapamycin resistance cell line, and a change in growth inhibitory effect is determined. Preferably, changes in growth inhibitory effect are determined using different concentrations of the candidate agent in conjunction with different concentrations of rapamycin such that one or more combinations of concentrations of the candidate agent and rapamycin which cause 50% inhibition, i.e., the IC₅₀, are determined.

In a preferred embodiment, an MTT proliferation assay (see, e.g., van de Loosdrechet, et al., 1994, J. Immunol. Methods 174: 311-320; Ohno et al., 1991, J. Immunol. Methods 145:199-203; Ferrari et al., 1990, J. Immunol. Methods 131: 165-172; Alley et al., 1988, Cancer Res. 48: 589-601; Carmichael et al., 1987, Cancer Res. 47:936-942; Gerlier et al., 1986, J. Immunol. Methods 65:55-63; Mosmann, 1983, J. Immunological Methods 65:55-63) is used to screen for a candidate agent in conjunction with rapamycin to inhibit the growth of rapamycin resistant cells. The cells are treated with chosen concentrations of the candidate agent and rapamycin for 4 to 72 hours. The cells are then incubated with a suitable amount of 3-(4,5-dimethylthiazol-2-yl)-2,5-diphenyltetrazolium bromide (MTT) for 1-8 hours such that viable cells convert MTT into an intracellular deposit of insoluble formazan. After removing the excess MTT contained in the supernatant, a suitable MTT solvent, e.g., a DMSO solution, is added to dissolved the formazan. The concentration of MTT, which is proportional to the number of viable cells, is then measured by determining the optical density at 570 μm. A plurality of different concentrations of the candidate agent can be assayed to allow the determination of the concentrations of the candidate agent and rapamycin which causes 50% inhibition.

### 5.4.2.3. Compounds Identified

The compounds identified in the screen include compounds that demonstrate the ability to selectively modulate the expression of RapR7 and regulate rapamycin resistance and/or tumorgenesis. These compounds include but are not limited to nucleic acid encoding RapR7 and homologues, analogues, and deletions thereof, as well as antisense, ribozyme, triple helix, dsRNAs, antibody, and polypeptide molecules and small organic or inorganic molecules.

The compounds identified in the screen also include compounds that modulate interaction of RapR7 with other proteins or molecules. In one embodiment, the compounds identified in the screen are compounds that modulate the interaction of a RapR7 protein with its interaction partner. In another embodiment, the compounds identified in the screen are compounds that modulate the interaction of a RapR7 protein with proteins or molecules that bind a PHD, a coiled-coil, a second peroximal domain, a nuclear localization domain, or a low complexity domain of the RapR7 protein. In another embodiment, the compounds identified in the screen are compounds that modulate the interaction of RapR7 gene with a transcription regulator.

### 5.4.3. Diagnostics

A variety of methods can be employed for the diagnostic and prognostic evaluation of rapamycin resistance and/or tumorgenesis resulting from defective regulation of RapR7, and for the identification of subjects having a predisposition to rapamycin resistance and/or tumorgenesis.

The invention provides methods for diagnosing in a mammal a cancer which is a result of defective regulation of a RapR7 gene or a predisposition to such a cancer. In one embodiment, the method comprises determining an expression level of the RapR7 gene in cells of the mammal, in which an expression level above a predetermined threshold level indicates that the mammal has or is predisposed of the cancer. Preferably, the predetermined threshold level is at least 2-fold, 4-fold, 8-fold, or 10-fold of the normal expression level of the RapR7 gene. In another embodiment, the invention provides a method for diagnosing in a mammal a cancer which is a result of defective regulation of a RapR7 gene or a predisposition to such a cancer comprising determining a level of abundance of a protein encoded by the RapR7 gene in cells of the mammal, in which a level of abundance of the protein above a predetermined threshold level indicates that the mammal has or is predisposed of the cancer. In still another embodiment, the invention provides a method for diagnosing the cancer comprising determining a level of activity of a protein encoded by the RapR7 gene in cells of the mammal, in which an activity level above a predetermined threshold level indicates that the mammal has or is predisposed of the cancer. As used herein, activities of a RapR7 protein include but not limited to its binding properties, e.g., binding specificity to a binding partner. Preferably, the predetermined threshold level of abundance or activity is at least 2-fold, 4-fold, 8-fold, or 10-fold of the normal level of abundance or activity of the RapR7 protein.

The invention also provides methods for evaluating rapamycin resistance in a cell. In one embodiment, the method comprises determining an expression level of a RapR7 gene in the cell, in which an expression level above a predetermined threshold level indicates that the cell is rapamycin resistant. Preferably, the predetermined threshold level is at least 2-fold, 4-fold, 8-fold, or 10-fold of the normal expression level of the RapR7 gene. In another embodiment, the invention provides a method for evaluating rapamycin resistance in a cell comprising determining a level of abundance of a protein encoded by a RapR7 gene in the cell, in which a level of abundance of the protein above a predetermined threshold level indicates that the cell is rapamycin resistant. In still another embodiment, the invention provides a method for evaluating rapamycin resistance in a cell comprising determining a level of activity of a protein encoded by the RapR7 gene in cells of the mammal, in which an activity level above a predetermined threshold level indicates that the cell is rapamycin resistant. Preferably, the predetermined threshold level of abundance or activity is at least 2-fold, 4-fold, 8-fold, or 10-fold of the normal level of abundance or activity of the RapR7 protein.

Such methods may, for example, utilize reagents such as the RapR7 gene nucleotide sequences described in Sections 5.1, and antibodies directed against RapR7 gene products, including peptide fragments thereof, as described, above, in Section 5.3. Specifically, such reagents may be used, for example, for: (1) the detection of the presence of RapR7 gene mutations, or the detection of either over- or under-expression of RapR7 gene mRNA relative to the normal expression level; and (2) the detection of either an over- or an under-abundance of RapR7 gene product relative to the normal RapR7 protein level.

The methods described herein may be performed, for example, by utilizing pre-packaged diagnostic kits comprising at least one specific RapR7 gene nucleic acid or anti-RapR7 gene antibody reagent described herein, which may be conveniently used, e.g., in clinical settings, to diagnose patients exhibiting RapR7 related disorder or abnormalities.

For the detection of RapR7 mutations, any nucleated cell can be used as a starting source for genomic nucleic acid. For the detection of RapR7 gene expression or RapR7 gene products, any cell type or tissue in which the RapR7 gene is expressed, such as, for example, hypothalamus cells, may be utilized.

Nucleic acid-based detection techniques are described, below, in Section 5.4.3.1. Peptide detection techniques are described, below, in Section 5.4.3.2.

### 5.4.3.1. Detection of Expression of RapR7 Gene

The expression of RapR7 gene in cells or tissues, e.g., the cellular level of RapR7 transcripts and/or the presence or absence of mutations, can be detected by utilizing a number of techniques. Nucleic acid from any nucleated cell can be used as the starting point for such assay techniques, and may be isolated according to standard nucleic acid preparation procedures which are well known to those of skill in the art. For example, the expression level of the RapR7 gene can determined by measuring the expression level of the RapR7 gene using one or more polynucleotide probes, each of which comprises a nucleotide sequence in the RapR7 gene. In one embodiment, the one or more polynucleotide probes comprise at least one polynucleotide probe comprising a nucleotide sequence within one of exons 1-11 of the RapR7 gene. In another embodiment, the one or more polynucleotide probes comprise at least one polynucleotide probe comprising a nucleotide sequence within an intron of the RapR7 gene. In particularly preferred embodiments of the invention, the methods is used to diagnose the cancer in a human.

DNA may be used in hybridization or amplification assays of biological samples to detect abnormalities involving RapR7 gene structure, including point mutations, insertions, deletions and chromosomal rearrangements. Such assays may include, but are not limited to, Southern analyses, single stranded conformational polymorphism analyses (SSCP), DNA microarray analyses, and PCR analyses.

Such diagnostic methods for the detection of RapR7 gene-specific mutations can involve for example, contacting and incubating nucleic acids including recombinant DNA molecules, cloned genes or degenerate variants thereof, obtained from a sample, e.g., derived from a patient sample or other appropriate cellular source, with one or more labeled nucleic acid reagents including recombinant DNA molecules, cloned genes or degenerate variants thereof, as described in Section 5.1, under conditions favorable for the specific annealing of these reagents to their complementary sequences within the RapR7 gene. Preferably, the lengths of these nucleic acid reagents are at least 15 to 30 nucleotides. After incubation, all non-annealed nucleic acids are removed from the nucleic acid:RapR7 molecule hybrid. The presence of nucleic acids which have hybridized, if any such molecules exist, is then detected. Using such a detection scheme, the nucleic acid from the cell type or tissue of interest can be immobilized, for example, to a solid support such as a membrane, or a plastic surface such as that on a microtiter plate or polystyrene beads. In this case, after incubation, non-annealed, labeled nucleic acid reagents of the type described in Section 5.1 are easily removed. Detection of the remaining, annealed, labeled RapR7 nucleic acid reagents is accomplished using standard techniques well-known to those in the art. The RapR7 gene sequences to which the nucleic acid reagents have annealed can be compared to the annealing pattern expected from a normal RapR7 gene sequence in order to determine whether a RapR7 gene mutation is present.

Alternative diagnostic methods for the detection of RapR7 gene specific nucleic acid molecules, in patient samples or other appropriate cell sources, may involve their amplification, e.g., by PCR (the experimental embodiment set forth in Mullis, K. B., 1987, U.S. Pat. No. 4,683,202), followed by the detection of the amplified molecules using techniques well known to those of skill in the art. The resulting amplified sequences can be compared to those which would be expected if the nucleic acid being amplified contained only normal copies of the RapR7 gene in order to determine whether a RapR7 gene mutation exists.

Among the RapR7 nucleic acid sequences which are preferred for such hybridization and/or PCR analyses are those which will detect the presence of the RapR7 gene splice site mutation.

Additionally, well-known genotyping techniques can be performed to identify individuals carrying RapR7 gene mutations. Such techniques include, for example, the use of restriction fragment length polymorphisms (RFLPs), which involve sequence variations in one of the recognition sites for the specific restriction enzyme used.

Additionally, improved methods for analyzing DNA polymorphisms which can be utilized for the identification of RapR7 gene mutations have been described which capitalize on the presence of variable numbers of short, tandemly repeated DNA sequences between the restriction enzyme sites. For example, Weber (U.S. Pat. No. 5,075,217, which is incorporated herein by reference in its entirety) describes a DNA marker based on length polymorphisms in blocks of (dC-dA)n-(dG-dT)n short tandem repeats. The average separation of (dC-dA)n-(dG-dT)n blocks is estimated to be 30,000-60,000 bp. Markers which are so closely spaced exhibit a high frequency co-inheritance, and are extremely useful in the identification of genetic mutations, such as, for example, mutations within the RapR7 gene, and the diagnosis of diseases and disorders related to RapR7 mutations.

Also, Caskey et al. (U.S. Pat. No. 5,364,759, which is incorporated herein by reference in its entirety) describe a DNA profiling assay for detecting short tri and tetra nucleotide repeat sequences. The process includes extracting the DNA of interest, such as the RapR7 gene, amplifying the extracted DNA, and labelling the repeat sequences to form a genotypic map of the individual's DNA.

The level of RapR7 gene expression can also be assayed. For example, RNA from a cell type or tissue known, or suspected, to express the RapR7 gene, such as MDCK cells or from a cell line which exhibits rapamycin resistance, may be isolated and tested utilizing hybridization or PCR techniques such as are described, above. The isolated cells can be derived from cell culture or from a patient. The analysis of cells taken from culture may be a necessary step in the assessment of cells to be used as part of a cell-based gene therapy technique or, alternatively, to test the effect of compounds on the expression of the RapR7 gene. Such analyses may reveal both quantitative and qualitative aspects of the expression pattern of the RapR7 gene, including activation or inactivation of RapR7 gene expression.

In one embodiment of such a detection scheme, a cDNA molecule is synthesized from an RNA molecule of interest (e.g., by reverse transcription of the RNA molecule into cDNA). A sequence within the cDNA is then used as the template for a nucleic acid amplification reaction, such as a PCR amplification reaction, or the like. The nucleic acid reagents used as synthesis initiation reagents (e.g., primers) in the reverse transcription and nucleic acid amplification steps of this method are chosen from among the RapR7 gene nucleic acid reagents described in Section 5.1. The preferred lengths of such nucleic acid reagents are at least 9-30 nucleotides. For detection of the amplified product, the nucleic acid amplification may be performed using radioactively or non-radioactively labeled nucleotides. Alternatively, enough amplified product may be made such that the product may be visualized by utilizing any suitable nucleic acid staining method, e.g., by standard ethidium bromide staining.

Additionally, it is possible to perform such RapR7 gene expression assays “in situ”, i.e., directly upon tissue sections (fixed and/or frozen) of patient tissue obtained from biopsies or resections, such that no nucleic acid purification is necessary. Nucleic acid reagents such as those described in Section 5.1 may be used as probes and/or primers for such in situ procedures (see, for example, Nuovo, G. J., 1992, “PCR In Situ Hybridization: Protocols And Applications”, Raven Press, NY).

Alternatively, if a sufficient quantity of the appropriate cells can be obtained, standard Northern analysis can be performed to determine the level of mRNA expression of the RapR7 gene.

The expression of RapR7 gene in cells or tissues, e.g., the cellular level of RapR7 transcripts and/or the presence or absence of mutations, can also be evaluated using DNA microarray technologies. In such technologies, one or more polynucleotide probes each comprising a sequence of the RapR7 gene are used to monitor the expression of the RapR7 gene. The present invention therefore provides DNA microarrays comprising polynucleotide probes comprising sequences of the RapR7 gene.

Any formats of DNA microarray technologies can be used in conjunction with the present invention. In one embodiment, spotted cDNA arrays are prepared by depositing PCR products of cDNA fragments, e.g., full length cDNAs, ESTs, etc., of the RapR7 gene onto a suitable surface (see, e.g., DeRisi et al., 1996, Nature Genetics 14:457-460; Shalon et al., 1996, Genome Res. 6:689-645; Schena et al., 1995, Proc. Natl. Acad. Sci. U.S.A. 93:10539-11286; and Duggan et al., Nature Genetics Supplement 21:10-14). In another embodiment, high-density oligonucleotide arrays containing oligonucleotides complementary to sequences of RapR7 gene are synthesized in situ on the surface by photolithographic techniques (see, e.g., Fodor et al., 1991, Science 251:767-773; Pease et al., 1994, Proc. Natl. Acad. Sci. U.S.A. 91:5022-5026; Lockhart et al., 1996, Nature Biotechnology 14:1675; McGall et al., 1996, Proc. Natl. Acad. Sci. U.S.A. 93:13555-13560; U.S. Pat. Nos. 5,578,832; 5,556,752; 5,510,270; 5,858,659; and 6,040,138). This format of microarray technology is particular useful for detection of single nucleotide polymorphisms (SNPs) (see, e.g., Hacia et al., 1999, Nat Genet. 22:164-7; Wang et al., 1998, Science 280:1077-82). In yet another embodiment, high-density oligonucleotide arrays containing oligonucleotides complementary to sequences of RapR7 gene are synthesized in situ on the surface by inkjet technologies (see, e.g., Blanchard, International Patent Publication WO 98/41531, published Sep. 24, 1998, Blanchard et al., 1996, Biosensors and Bioelectronics 11:687-690; Blanchard, 1998, in Synthetic DNA Arrays in Genetic Engineering, Vol. 20, J. K. Setlow, Ed., Plenum Press, New York at pages 111-123). In still another embodiment, DNA microarrays that allow electronic stringency control can be used in conjunction with polynucleotide probes comprising sequences of the RapR7 gene (see, e.g., U.S. Pat. No. 5,849,486).

### 5.4.3.2. Detection OF RapR7 Gene Products

Antibodies directed against wild type or mutant RapR7 gene products or conserved variants or peptide fragments thereof, which are discussed, above, in Section 5.3, may also be used as diagnostics and prognostics of rapamycin resistance and/or tumorgenesis, as described herein. Such diagnostic methods, may be used to detect abnormalities in the level of RapR7 gene expression, or abnormalities in the structure and/or temporal, tissue, cellular, or subcellular location of RapR7 gene product. In exemplary embodiments of the invention, the protein detected is a human RapR7 protein as depicted in SEQ ID NO:6 or 7, or a murine RapR7 protein as depicted in SEQ ID NO:3 or 4.

Because evidence disclosed herein indicates that the RapR7 gene product is an intracellular gene product, the antibodies and immunoassay methods described below have important in vitro applications in assessing the efficacy of treatments for disorders resulting from defective regulation of RapR7 gene such as infectious diseases, immunodeficiencies, autoimmune diseases, inflammatory diseases, and proliferative diseases. Antibodies, or fragments of antibodies, such as those described below, may be used to screen potentially therapeutic compounds in vitro to determine their effects on RapR7 gene expression and RapR7 peptide production. The compounds which have beneficial effects on disorders related to defective regulation of RapR7 can be identified, and a therapeutically effective dose determined.

In vitro immunoassays may also be used, for example, to assess the efficacy of cell-based gene therapy for disorders related to defective regulation of RapR7. Antibodies directed against RapR7 peptides may be used in vitro to determine the level of RapR7 gene expression achieved in cells genetically engineered to produce RapR7 peptides. Given that evidence disclosed herein indicates that the RapR7 gene product is an intracellular gene product, such an assessment is, preferably, done using cell lysates or extracts. Such analysis will allow for a determination of the number of transformed cells necessary to achieve therapeutic efficacy in vivo, as well as optimization of the gene replacement protocol.

The tissue or cell type to be analyzed will generally include those which are known, or suspected, to express the RapR7 gene, such as, for example, hypothalamic cells. The protein isolation methods employed herein may, for example, be such as those described in Harlow and Lane (Harlow, E. and Lane, D., 1988, “Antibodies: A Laboratory Manual”, Cold Spring Harbor Laboratory Press, Cold Spring Harbor, N.Y.), which is incorporated herein by reference in its entirety. The isolated cells can be derived from cell culture or from a patient. The analysis of cell taken from culture may be a necessary step in the assessment of cells to be used as part of a cell-based gene therapy technique or, alternatively, to test the effect of compounds on the expression of the RapR7 gene.

Preferred diagnostic methods for the detection of RapR7 gene products or conserved variants or peptide fragments thereof, may involve, for example, immunoassays wherein the RapR7 gene products or conserved variants or peptide fragments are detected by their interaction with an anti-RapR7 gene product-specific antibody.

For example, antibodies, or fragments of antibodies, such as those described, above, in Section 5.3, useful in the present invention may be used to quantitatively or qualitatively detect the presence of RapR7 gene products or conserved variants or peptide fragments thereof. This can be accomplished, for example, by immunofluorescence techniques employing a fluorescently labeled antibody (see below, this Section) coupled with light microscopic, flow cytometric, or fluorimetric detection. Such techniques are especially preferred if such RapR7 gene products are expressed on the cell surface.

The antibodies (or fragments thereof) useful in the present invention may, additionally, be employed histologically, as in immunofluorescence or immunoelectron microscopy, for in situ detection of RapR7 gene products or conserved variants or peptide fragments thereof. In situ detection may be accomplished by removing a histological specimen from a patient, and applying thereto a labeled antibody of the present invention. The antibody (or fragment) is preferably applied by overlaying the labeled antibody (or fragment) onto a biological sample. Through the use of such a procedure, it is possible to determine not only the presence of the RapR7 gene product, or conserved variants or peptide fragments, but also its distribution in the examined tissue. Using the present invention, those of ordinary skill will readily perceive that any of a wide variety of histological methods (such as staining procedures) can be modified in order to achieve such in situ detection.

Immunoassays for RapR7 gene products or conserved variants or peptide fragments thereof will typically comprise incubating a sample, such as a biological fluid, a tissue extract, freshly harvested cells, or lysates of cells which have been incubated in cell culture, in the presence of a detectably labeled antibody capable of identifying RapR7 gene products or conserved variants or peptide fragments thereof, and detecting the bound antibody by any of a number of techniques well-known in the art.

The biological sample may be brought in contact with and immobilized onto a solid phase support or carrier such as nitrocellulose, or other solid support which is capable of immobilizing cells, cell particles or soluble proteins. The support may then be washed with suitable buffers followed by treatment with the detectably labeled RapR7 protein specific antibody. The solid phase support may then be washed with the buffer a second time to remove unbound antibody. The amount of bound label on solid support may then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tub, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

The binding activity of a given lot of anti-RapR7 gene product antibody may be determined according to well known methods. Those skilled in the art will be able to determine operative and optimal assay conditions for each determination by employing routine experimentation.

One of the ways in which the RapR7 gene peptide-specific antibody can be detectably labeled is by linking the same to an enzyme and use in an enzyme immunoassay (EIA) (Voller, A., “The Enzyme Linked Immunosorbent Assay (ELISA)”, 1978, Diagnostic Horizons 2:1-7, Microbiological Associates Quarterly Publication, Walkersville, Md.); Voller, A. et al., 1978, J. Clin. Pathol. 31:507-520; Butler, J. E., 1981, Meth. Enzymol. 73:482-523; Maggio, E. (ed.), 1980, Enzyme Immunoassay, CRC Press, Boca Raton, Fla.; Ishikawa, E. et al., (eds.), 1981, Enzyme Immunoassay, Kgaku Shoin, Tokyo). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by calorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect RapR7 gene peptides through the use of a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

### 5.4.4. Methods of Regulating Expression of RapR7 Gene

A variety of therapeutic approaches may be used in accordance with the invention to modulate expression of the RapR7 gene in vivo. For example, antisense DNA molecules may be engineered and used to block translation of RapR7 mRNA in vivo. Alternatively, ribozyme molecules may be designed to cleave and destroy the RapR7 mRNAs in vivo. In another alternative, oligonucleotides designed to hybridize to the 5′ region of the RapR7 gene (including the region upstream of the coding sequence) and form triple helix structures may be used to block or reduce transcription of the RapR7 gene. Oligonucleotides can also be designed to hybridize and form triple helix structures with the binding site of a negative regulator so as to block the binding of the negative regulator and to enhance the transcription of the RapR7 gene. In yet another alternative, nucleic acid encoding the full length wild-type RapR7 message may be introduced in vivo into cells which otherwise would be unable to produce the wild-type RapR7 gene product in sufficient quantities or at all. In yet another embodiment, a heterologous regulatory element may be inserted before the coding sequence of a RapR7 gene, such that it is operatively linked with and activates expression of the endogenous RapR7 gene.

In a preferred embodiment, the antisense, ribozyme, and triple helix nucleotides are designed to inhibit the translation or transcription of one or more of RapR7 isoforms with minimal effects on the expression of other genes that may share one or more sequence motif with a RapR7. To accomplish this, the oligonucleotides used should be designed on the basis of relevant sequences unique to RapR7.

For example, and not by way of limitation, the oligonucleotides should not fall within those region where the nucleotide sequence of RapR7 is most homologous to that of other genes, e.g., a PHD domain. Instead, it is preferred that the oligonucleotides fall within the portion of the sequence of RapR7 that does not encode a PHD domain. In the case of antisense molecules, it is preferred that the sequence be chosen from the list above. It is also preferred that the sequence be at least 18 nucleotides in length in order to achieve sufficiently strong annealing to the target mRNA sequence to prevent translation of the sequence. Izant et al., 1984, Cell, 36:1007-1015; Rosenberg et al., 1985, Nature, 313:703-706.

In the case of the “hammerhead” type of ribozymes, it is also preferred that the target sequences of the ribozymes be chosen from the list above. Ribozymes are RNA molecules which possess highly specific endoribonuclease activity. Hammerhead ribozymes comprise a hybridizing region which is complementary in nucleotide sequence to at least part of the target RNA, and a catalytic region which is adapted to cleave the target RNA. The hybridizing region contains nine (9) or more nucleotides. Therefore, the hammerhead ribozymes of the present invention have a hybridizing region which is complementary to the sequences listed above and is at least nine nucleotides in length. The construction and production of such ribozymes is well known in the art and is described more fully in Haseloff et al., 1988, Nature, 334:585-591.

The ribozymes of the present invention also include RNA endoribonucleases (hereinafter “Cech-type ribozymes”) such as the one which occurs naturally in Tetrahymena Thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. WO 88/04300 by University Patents Inc.; Been et al., 1986, Cell, 47:207-216). The Cech endoribonucleases have an eight base pair active site which hybridizes to a target RNA sequence whereafter cleavage of the target RNA takes place.

In the case of oligonucleotides that hybridize to and form triple helix structures at the 5′ terminus of the RapR7 gene and can be used to block transcription, it is preferred that they be complementary to those sequences in the 5′ terminus of RapR7 which are not present in other RapR7 related genes. It is also preferred that the sequences not include those regions of the RapR7 promoter which are even slightly homologous to that of other RapR7 related genes. The foregoing compounds can be administered by a variety of methods which are known in the art including, but not limited to the use of liposomes as a delivery vehicle. Naked DNA or RNA molecules may also be used where they are in a form which is resistant to degradation such as by modification of the ends, by the formation of circular molecules, or by the use of alternate bonds including phosphothionate and thiophosphoryl modified bonds. In addition, the delivery of nucleic acid may be by facilitated transport where the nucleic acid molecules are conjugated to poly-lysine or transferrin. Nucleic acid may also be transported into cells by any of the various viral carriers, including but not limited to, retrovirus, vaccinia, AAV, and adenovirus.

Alternatively, a recombinant nucleic acid molecule which encodes, or is, such antisense, ribozyme, triple helix, or RapR7 molecule can be constructed. This nucleic acid molecule may be either RNA or DNA. If the nucleic acid encodes an RNA, it is preferred that the sequence be operatively attached to a regulatory element so that sufficient copies of the desired RNA product are produced. The regulatory element may permit either constitutive or regulated transcription of the sequence. In vivo, that is, within the cells or cells of an organism, a transfer vector such as a bacterial plasmid or viral RNA or DNA, encoding one or more of the RNAs, may be transfected into cells e.g. (Llewellyn et al., 1987, J. Mol. Biol., 195:115-123; Hanahan et al. 1983, J. Mol. Biol., 166:557-580). Once inside the cell, the transfer vector may replicate, and be transcribed by cellular polymerases to produce the RNA or it may be integrated into the genome of the host cell. Alternatively, a transfer vector containing sequences encoding one or more of the RNAs may be transfected into cells or introduced into cells by way of micromanipulation techniques such as microinjection, such that the transfer vector or a part thereof becomes integrated into the genome of the host cell.

RNA interference (RNAi) can also be used to block expression of RapR7 (Guo et al., 1995, Cell 81:611-620; Fire et al., 1998, Nature 391:806-811; Grant, 1999, Cell 96:303-306; Tabara et al., 1999, Cell 99:123-132; Zamore et al., 2000, Cell 101:25-33; Bass, 2000, Cell 101:235-238; Petcherski et al., 2000, Nature 405:364-368; Elbashir et al., Nature 411:494-498; Paddison et al., Proc. Natl. Acad. Sci. USA 99:1443-1448). In one embodiment, double-stranded RNA molecules of 21-23 nucleotides which hybridize to a homologous region of mRNAs transcribed from the RapR7 gene are used to degrade the mRNAs, thereby “silence” the expression of the RapR7 gene. Preferably, the dsRNAs have a hybridizing region, e.g., a 19-nucleotide double-stranded region, which is complementary to a sequence of the coding sequence of the RapR7 gene. Any siRNA targeting an appropriate coding sequence of a RapR7 gene, e.g., a human RapR7 gene, can be used in the invention. As an exemplary embodiment, 21-nucleotide double-stranded siRNAs targeting the coding regions of RapR7 gene are designed according to standard selection rules (see, e.g., Elbashir et al., 2002, Methods 26:199-213, which is incorporated herein by reference in its entirety).

Any standard method for introducing nucleic acids into cells can be used. In one embodiment, gene silencing is induced by presenting the cell with the siRNA targeting the RapR7 gene (see, e.g., Elbashir et al., 2001, Nature 411, 494-498; Elbashir et al., 2001, Genes Dev. 15, 188-200, all of which are incorporated by reference herein in their entirety). The siRNAs can be chemically synthesized, or derived from cleavage of double-stranded RNA by recombinant Dicer. Another method to introduce a double stranded DNA (dsRNA) for silencing of the RapR7 gene is shRNA, for short hairpin RNA (see, e.g., Paddison et al., 2002, Genes Dev. 16, 948-958; Brummelkamp et al., 2002, Science 296, 550-553; Sui, G. et al. 2002, Proc. Natl. Acad. Sci. USA 99, 5515-5520, all of which are incorporated by reference herein in their entirety). In this method, an siRNA targeting RapR7 gene is expressed from a plasmid (or virus) as an inverted repeat with an intervening loop sequence to form a hairpin structure. The resulting RNA transcript containing the hairpin is subsequently processed by Dicer to produce siRNAs for silencing. Plasmid-based shRNAs can be expressed stably in cells, allowing long-term gene silencing in cells both in vitro and in vivo (see, McCaffrey et al. 2002, Nature 418, 38-39; Xia et al., 2002, Nat. Biotech. 20, 1006-1010; Lewis et al., 2002, Nat. Genetics 32, 107-108; Rubinson et al., 2003, Nat. Genetics 33, 401-406; Tiscornia et al., 2003, Proc. Natl. Acad. Sci. USA 100, 1844-1848, all of which are incorporated by reference herein in their entirety). SiRNAs targeting the RapR7 gene can also be delivered to an organ or tissue in a mammal, such a human, in vivo (see, e.g., Song et al. 2003, Nat. Medicine 9, 347-351; Sorensen et al., 2003, J. Mol. Biol. 327, 761-766; Lewis et al., 2002, Nat. Genetics 32, 107-108, all of which are incorporated by reference herein in their entirety). In this method, a solution of siRNA is injected intravenously into the mammal. The siRNA can then reach an organ or tissue of interest and effectively reduce the expression of the target gene in the organ or tissue of the mammal.

The expression of RapR7 genes can also be activated or enhanced. In one embodiment, a heterologous regulatory element may be inserted before the coding sequence of a RapR7 gene, such that it is operatively linked with and activates expression of the endogenous RapR7 gene, using techniques, such as targeted homologous recombination, which are well known to those of skill in the art, and described e.g., in Chappel, U.S. Pat. No. 5,272,071; PCT publication No. WO 91/06667, published May 16, 1991; Skoultchi U.S. Pat. No. 5,981,214; Treco et al U.S. Pat. No. 5,968,502 and PCT publication No. WO 94/12650, published Jun. 9, 1994. Alternatively, non-targeted e.g., non-homologous recombination techniques which are well-known to those of skill in the art and described, e.g., in PCT publication No. WO 99/15650, published Apr. 1, 1999, may be used.

In another embodiment, the expression of a RapR7 gene is enhanced by blocking the binding of a negative regulator (i.e., a repressor). Any agent that binds to such site and inhibit the binding of a regulator molecule, including but not limited to peptides or nucleic acid molecules, can be used for this purpose.

### 5.4.5. Gene Therapy Based on RapR7 Gene

A variety of gene therapy approaches may be used in accordance with the invention to modulate expression of the RapR7 gene in vivo. In yet another alternative, nucleic acid encoding the full length wild-type RapR7 message may be introduced in vivo into cells which otherwise would be unable to produce the wild-type RapR7 gene product in sufficient quantities or at all.

In a specific embodiment, nucleic acids comprising a sequence encoding a RapR7 or functional derivative thereof, are administered to promote a RapR7 function, by way of gene therapy. Gene therapy refers to therapy performed by the administration of a nucleic acid to a subject. In this embodiment of the invention, the nucleic acid produces its encoded protein that mediates a therapeutic effect by promoting a RapR7 function.

Any of the methods for gene therapy available in the art can be used according to the present invention. Exemplary methods are described below.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215). Methods commonly known in the art of recombinant DNA technology which can be used are described in Ausubel et al. (eds.), 1993, Current Protocols in Molecular Biology, John Wiley & Sons, New York; and Kriegler, 1990, Gene Transfer and Expression, A Laboratory Manual, Stockton Press, New York.

In a preferred aspect, the therapeutic comprises a RapR7 nucleic acid that is part of an expression vector that expresses a RapR7 or fragment or chimeric protein thereof in a suitable host. In particular, such a nucleic acid has a promoter operably linked to the RapR7 coding region, said promoter being inducible or constitutive, and, optionally, tissue-specific. In another particular embodiment, a nucleic acid molecule is used in which the RapR7 coding sequences and any other desired sequences are flanked by regions that promote homologous recombination at a desired site in the genome, thus providing for intrachromosomal expression of the RapR7 nucleic acid (see e.g., Koller and Smithies, 1989, Proc. Natl. Acad. Sci. U.S.A. 86:8932-8935; Zijlstra et al., 1989, Nature 342:435-438).

Delivery of the nucleic acid into a patient may be either direct, in which case the patient is directly exposed to the nucleic acid or nucleic acid-carrying vector, or indirect, in which case, cells are first transformed with the nucleic acid in vitro, then transplanted into the patient. These two approaches are known, respectively, as in vivo or ex vivo gene therapy.

In a specific embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering it in linkage to a peptide which is known to enter the nucleus, by administering it in linkage to a ligand subject to receptor-mediated endocytosis (see e.g., Wu and Wu, 1987, J. Biol. Chem. 262:4429-4432) (which can be used to target cell types specifically expressing the receptors), etc. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992 (Wu et al.); WO 92/22635 dated Dec. 23, 1992 (Wilson et al.); WO92/20316 dated Nov. 26, 1992 (Findeis et al.); WO93/14188 dated Jul. 22, 1993 (Clarke et al.), WO 93/20221 dated Oct. 14, 1993 (Young)). Alternatively, the nucleic acid can be introduced intracellularly and incorporated within host cell DNA for expression, by homologous recombination (Koller and Smithies, 1989, Proc. Natl. Acad. Sci. U.S.A. 86:8932-8935; Zijlstra et al., 1989, Nature 342:435-438).

In a specific embodiment, a viral vector that contains the RapR7 nucleic acid is used. For example, a retroviral vector can be used (see Miller et al., 1993, Meth. Enzymol. 217:581-599). These retroviral vectors have been modified to delete retroviral sequences that are not necessary for packaging of the viral genome and integration into host cell DNA.

The RapR7 nucleic acid to be used in gene therapy is cloned into the vector, which facilitates delivery of the gene into a patient. More detail about retroviral vectors can be found in Boesen et al., 1994, Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. Genet. and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson (1993, Current Opinion in Genetics and Development 3:499-503) present a review of adenovirus-based gene therapy. Bout et al. (1994, Human Gene Therapy 5:3-10) demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; and Mastrangeli et al., 1993, J. Clin. Invest. 91:225-234.

Adeno-associated virus (AAV) has also been proposed for use in gene therapy (Walsh et al., 1993, Proc. Soc. Exp. Biol. Med. 204:289-300.

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. In a preferred embodiment, epithelial cells are injected, e.g., subcutaneously. In another embodiment, recombinant skin cells may be applied as a skin graft onto the patient. Recombinant blood cells (e.g., hematopoietic stem or progenitor cells) are preferably administered intravenously. The amount of cells envisioned for use depends on the desired effect, patient state, etc., and can be determined by one skilled person in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, a RapR7 nucleic acid is introduced into the cells such that it is expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention. Such stem cells include but are not limited to hematopoietic stem cells (HSC), stem cells of epithelial tissues such as the skin and the lining of the gut, embryonic heart muscle cells, liver stem cells (PCT Publication WO 94/08598, dated Apr. 28, 1994), and neural stem cells (Stemple and Anderson, 1992, Cell 71:973-985).

Epithelial stem cells (ESCs) or keratinocytes can be obtained from tissues such as the skin and the lining of the gut by known procedures (Rheinwald, 1980, Meth. Cell Bio. 21A:229). In stratified epithelial tissue such as the skin, renewal occurs by mitosis of stem cells within the germinal layer, the layer closest to the basal lamina. Stem cells within the lining of the gut provide for a rapid renewal rate of this tissue. ESCs or keratinocytes obtained from the skin or lining of the gut of a patient or donor can be grown in tissue culture (Rheinwald, 1980, Meth. Cell Bio. 21A:229; Pittelkow and Scott, 1986, Mayo Clinic Proc. 61:771). If the ESCs are provided by a donor, a method for suppression of host versus graft reactivity (e.g., irradiation, drug or antibody administration to promote moderate immunosuppression) can also be used.

With respect to hematopoietic stem cells (HSC), any technique which provides for the isolation, propagation, and maintenance in vitro of HSC can be used in this embodiment of the invention. Techniques by which this may be accomplished include (a) the isolation and establishment of HSC cultures from bone marrow cells isolated from the future host, or a donor, or (b) the use of previously established long-term HSC cultures, which may be allogeneic or xenogeneic. Non-autologous HSC are used preferably in conjunction with a method of suppressing transplantation immune reactions of the future host/patient. In a particular embodiment of the present invention, human bone marrow cells can be obtained from the posterior iliac crest by needle aspiration (see e.g., Kodo et al., 1984, J. Clin. Invest. 73:1377-1384). In a preferred embodiment of the present invention, the HSCs can be made highly enriched or in substantially pure form. This enrichment can be accomplished before, during, or after long-term culturing, and can be done by any techniques known in the art. Long-term cultures of bone marrow cells can be established and maintained by using, for example, modified Dexter cell culture techniques (Dexter et al., 1977, J. Cell Physiol. 91:335) or Witlock-Witte culture techniques (Witlock and Witte, 1982, Proc. Natl. Acad. Sci. U.S.A. 79:3608-3612).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

Additional methods that can be adapted for use to deliver a nucleic acid encoding a RapR7 or functional derivative thereof are described below.

### 5.4.6. Methods of Regulating Activity of RapR7 Protein and/or RapR7 Pathways

The activity of RapR7 protein can be regulated by modulating the interaction of RapR7 protein with its binding partners. In one embodiment, agents, e.g., antibodies, small organic or inorganic molecules, can be used to inhibit binding of a RapR7 binding partner such that rapamycin resistance and/or tumorgenesis is regulated. In another embodiment, agents, e.g., antibodies, small organic or inorganic molecules, can be used to inhibit the activity of a protein in a RapR7 protein regulatory pathway, including but not limited to cyclin D1 or cdc2, such that rapamycin resistance and/or tumorgenesis is regulated.

### 5.4.7. Cancer Therapy by Targeting RapR7 Gene, Gene Product, and/or Other Related Cellular Molecules

The methods and/or compositions described above for modulating RapR7 expression and/or activity may be used to treat patients who have a cancer as a result of defective regulation of a RapR7 gene. The methods and/or compositions may also be used in conjunction with rapamycin for treatment of a patient having a cancer which exhibits RapR7 mediated rapamycin resistance and/or tumorgenesis. Such therapies may be used to treat cancers, including but not limted to, rhabdomyosarcoma, neuroblastoma and glioblastoma, small cell lung cancer, osteoscarcoma, pancreatic cancer, breast and prostate cancer, murine melanoma and leukemia, and B-cell lymphoma.

In preferred embodiments, the methods and/or compositions of the invention are used in conjunction with rapamycin for treatment of a patient having a cancer which exhibits RapR7 mediated rapamycin resistance and/or tumorgenesis. In such embodiments, the expression and/or activity of RapR7 are modulated to confer cancer cells sensitivity to rapamycin, thereby conferring or enhancing the efficacy of rapamycin therapy.

In a combination therapy, one or more compositions of the present invention can be administered before, at the same time of, or after the administration of rapamycin. In one embodiment, the compositions of the invention are administered before the administration rapamycin. The time intervals between the administration of the compositions of the invention and rapamycin can be determined by routine experiments that are familiar to one skilled person in the art. In one embodiment, rapamycin is given after the RapR7 protein level reaches a desirable threshold. The level of RapR7 protein can be determined by using any techniques described supra.

In another embodiment, the compositions of the invention are administered at the same time with rapamycin.

In still another embodiment, one or more of the compositions of the invention are also administered after the administration of rapamycin. Such administration can be beneficial especially when rapamycin has a longer half life than that of the one or more of the compositions of the invention used in the treatment.

It will be apparent to one skilled person in the art that any combination of different timing of the administration of the compositions of the invention and rapamycin can be used. For example, when rapamycin has a longer half life than that of the composition of the invention, it is preferable to administer the compositions of the invention before and after the administration of the rapamycin.

The frequency or intervals of administration of the compositions of the invention depends on the desired RapR7 level, which can be determined by any of the techniques described supra. The administration frequency of the compositions of the invention can be increased or decreased when the RapR7 protein level changes either higher or lower from the desired level.

The effects or benefits of administration of the compositions of the invention alone or in conjunction with rapamysin can be evaluated by any methods known in the art, e.g., by methods that are based on measuring the survival rate, side effects, dosage requirement of rapamycin, or any combinations thereof. If the administration of the compositions of the invention achieves any one or more of the benefits in a patient, such as increasing the survival rate, decreasing side effects, lowing the dosage requirement for rapamycin, the compositions of the invention are said to have augmented the rapamycin therapy, and the method is said to have efficacy.

### 5.5. Pharmaceutical Formulations and Routes of Administration

The compounds that are determined to affect RapR7 gene expression or gene product activity can be administered to a patient at therapeutically effective doses to treat or ameliorate disorders related to defective regulation of RapR7. A therapeutically effective dose refers to that amount of the compound sufficient to result in amelioration of rapamycin resistance and/or inhibition of the growth of cancer cells.

### 5.5.1. Effective Dose

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### 5.5.2. Formulations and Use

Pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g. almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

### 5.5.3. Routes of Administration

Suitable routes of administration may, for example, include oral, rectal, transmucosal, transdermal, or intestinal administration; parenteral delivery, including intramuscular, subcutaneous, intramedullary injections, as well as intrathecal, direct intraventricular, intravenous, intraperitoneal, intranasal, or intraocular injections.

Alternately, one may administer the compound in a local rather than systemic manner, for example, via injection of the compound directly into an affected area, often in a depot or sustained release formulation.

Furthermore, one may administer the drug in a targeted drug delivery system, for example, in a liposome coated with an antibody specific for affected cells. The liposomes will be targeted to and taken up selectively by the cells.

### 5.5.4. Packaging

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labelled for treatment of an indicated condition. Suitable conditions indicated on the label may include treatment of a disease such as one characterized by insufficient, aberrant, or excessive RapR7 activity.

### 6. EXAMPLE

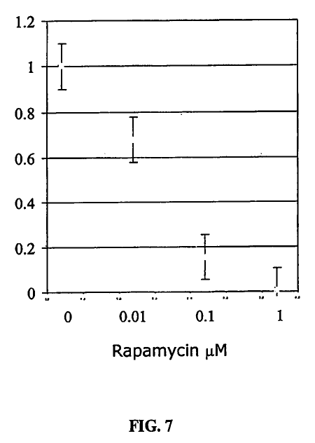

A mouse neuroblastoma cell line N2a cells (ATCC) were used for treatment with rapamycin. N2a cells are sensitive to rapamycin mediated cell growth inhibition at 10 nM (See, FIG. 7, MTT assay, a proliferation assay) and were used as target cells for the random homozygous knockout (RHKO) procedure to identify the genes and genetic pathways whose inactivation result in N2a cells resistant to rapamycin mediated cell growth inhibition.

N2a cells were transfected with a vector carrying a transactivor (FIG. 9D). The vector was modified from the expression regulator vector of the Tet-off™ system (Clontech, Palo Alto, Calif.). Several cell clones were generated. One of these clones (clone 44) shown strong transactivator activity was used as target cells for the infection of a retroviral vector (FIG. 9C) carrying a gene search construct as depicted in FIGS. 9A and 9C. The infected N2a cells were selected with puromycin within 4 days for cells undergone RHKO. Since the expression of puromycin is dependent on transactivator and can be suppressed by addition of doxcycline in the culture medium, selection of RHKO clones was carried out in culture medium without doxcycline. One week later, a RHKO library of more than 100,000 puromycin resistant cells was generated. The cells were treated with 1 μM of rapamycin (at this concentration all N2a cells were either killed or growth-inhibited by rapamycin). Fourteen days later, 8 rapamycin resistant colonies were isolated and expanded into cell lines.

To confirm RHKO-dependent rapamycin resistance of the cell lines, cells were assayed for their resistance to rapamycin in the present and in the absence of doxcycline. RHKO dependent rapamycin resistance should be reverted to rapamycin sensitive in the presence of doxcycline. Clone 7 (RapR7 clone, see FIGS. 8A-8B) showed reversibility in the presence of doxccycline, indicating that random homozygous knockout of specific genes in the three clones resulted in the cellular resistant to rapamycin mediated growth inhibition. FIGS. 8A-8B illustrate reversible inhibition by rapamycin in RapR7 clone (MTT proliferation assay). 8A: Solid bar, measurement when the knockout construct is expressed; shaded bar, measurement when the expression of the knockout construct is suppressed; and open bar, control. 8B: Calculated reversibility R according to Equation 1. FIG. 8C illustrates a RapR7 colony after 12 days of infection with an RHKO gene search vector.

FIGS. 8D-8E illustrate effects of expression of RapR7 gene on markers of G1 arrest in RapR7 clone. FIGS. 8D-8E show that inactivation of RapR7 gene increases the cellular level of Cyclin D1 (FIG. 8D) and cdc2 (FIG. 8E) but does not affect the cellular level of p70S6 (FIG. 8D). CDC2 protein kinase (also termed p34 protein kinase) which is activated by forming a complex with cyclin B and is required for the G2/M transition, i.e., the transition from G2 phase to mitosis, of the cell cycle. Reduction in CDC2 expression and/or activity has been shown to lead to G2/M arrest. These results indicate that RapR7 gene plays a role in cell tumorgenesis.

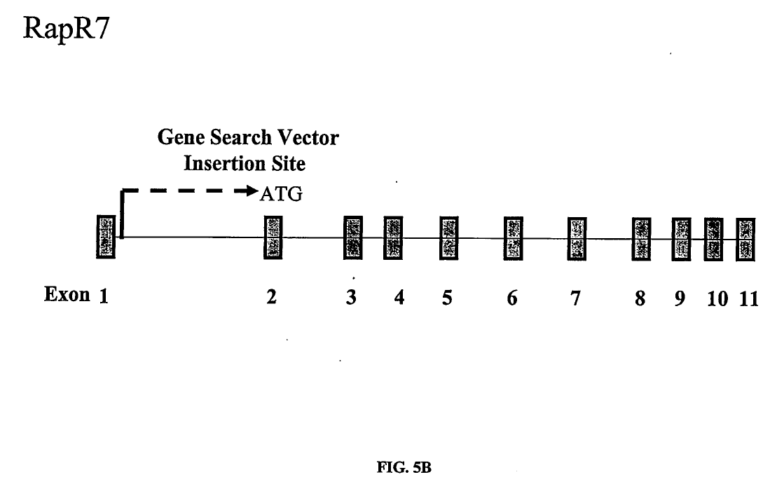

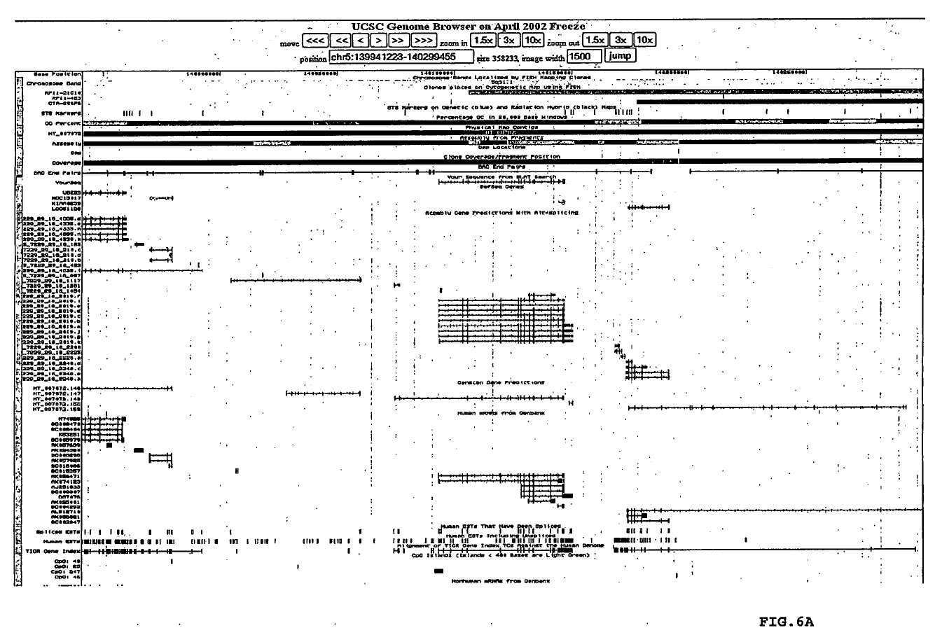

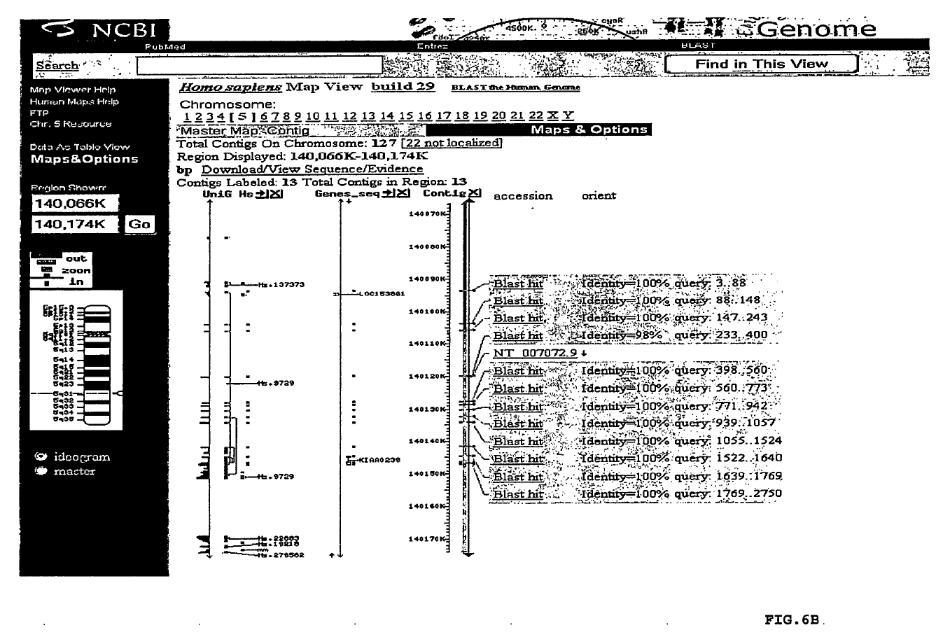

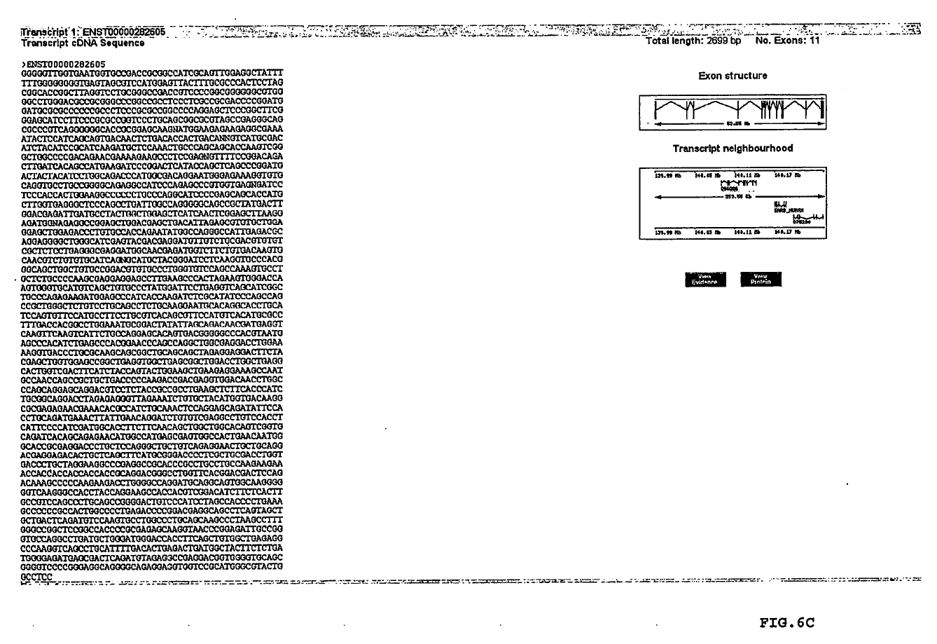

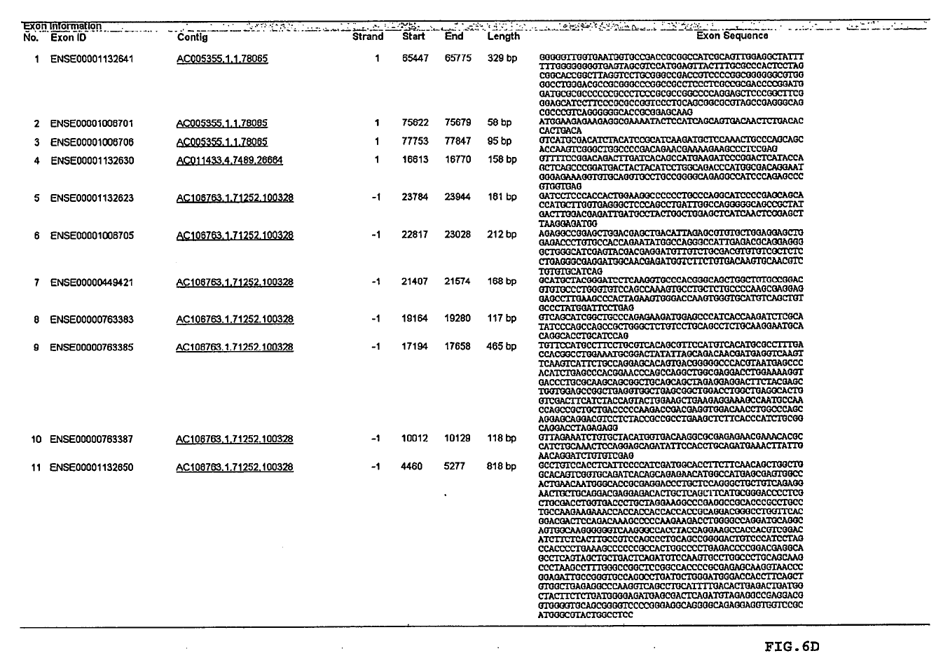

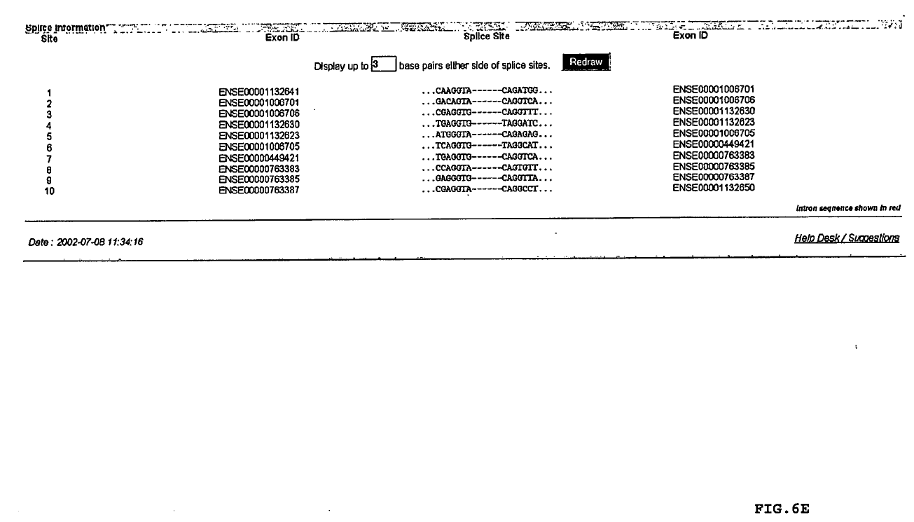

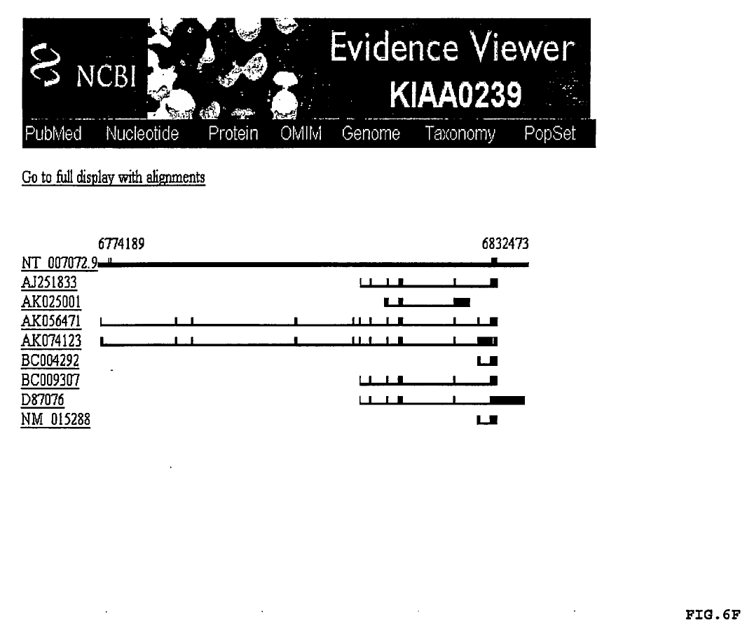

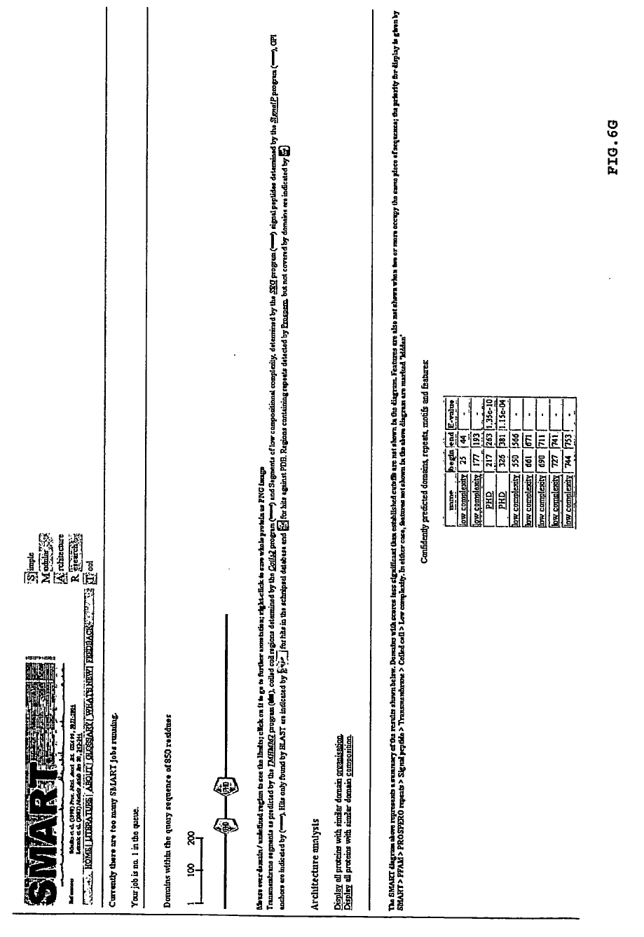

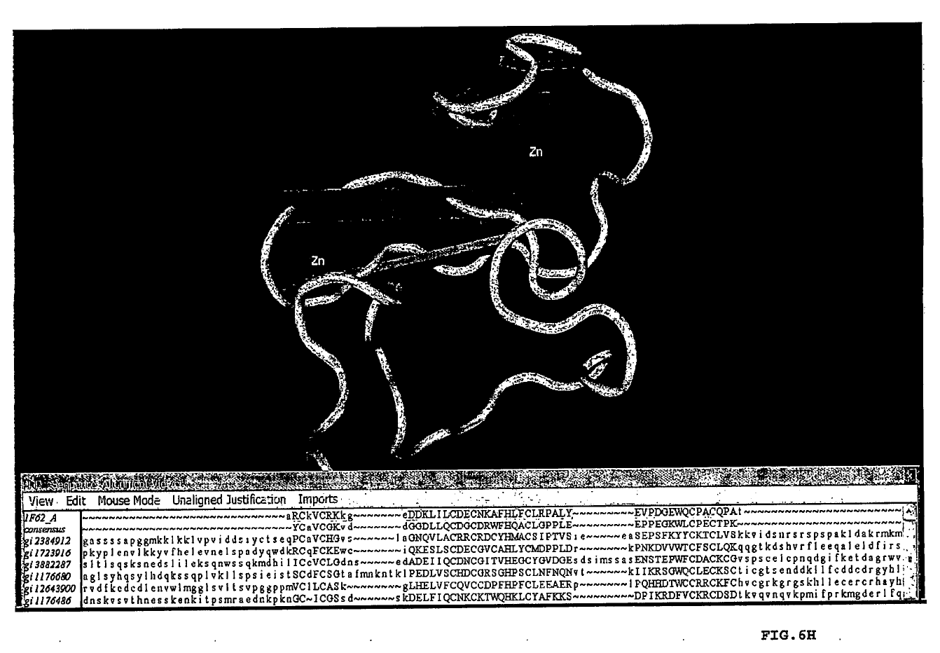

Southern blotting analysis of RapR7 showed that the clone contains a single copy integration of the gene search vector, indicating only one gene has been inactivated by RHKO in each clone. The DNA sequences of the gene search vector were used to clone the genomic DNA fragment flanking the integrated gene search construct. HindIII was used to digest the genomic DNA. A 4 kb plasmid carrying a 1.5 kb genomic DNA flanking the RHKO insertion site was obtained. Two plasmids (designated as RapR71 and RapR72, respectively) were isolated and sequenced using an oligo primer from gene search vector. FIG. 1A depicts a nucleotide sequence (SEQ ID NO:1) obtained from sequencing one of the plasmid. DNA sequences of the flanking genomic DNA fragments were obtained and used to search databases to identify the genes. FIG. 1B depicts an alignment of the sequences obtained from the two plasmids. FIG. 5D depict the genomic location of the RHKO insertion site. The RHKO vector is inserted in the intron between exon 1 and exon 2 of the murine RapR7 gene. FIG. 2A depict the nucleotide sequences of the transcript cDNA (SEQ ID NO:2) and the protein (SEQ ID NO:3).

### 7. REFERENCES CITED

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to e limited only by the terms of the appended claims along with the full scope of equivalents to which such claims are entitled.

What is claimed is:

1. A purified mammalian RapR7 protein.

2. The protein of claim 1 which comprises the amino acid sequence substantially as set forth in SEQ ID NO:4 or 7.

3. A purified protein encoded by a nucleic acid capable of hybridizing to a DNA comprising a sequence consisting of the coding region of SEQ ID NO:2 or 5.

4. A purified derivative or analog of the protein of claim 1, which displays one or more functional activities of a mammalian RapR7 protein.

5. The derivative or analog of claim 4 which is capable of binding to an antibody directed against a mammalian RapR7 protein.

6. A purified fragment of a mammalian RapR7 protein.

7. The fragment of claim 6, wherein said fragment comprises a PHD domain of a mammalian RapR7 protein.

8. The fragment of claim 7, wherein said mammalian RapR7 protein is a human RapR7 protein, and wherein said fragment comprises amino acids 217-263 or 326-381 of said RapR7 protein.

9. The fragment of claim 6, wherein said fragment comprises a coiled-coil domain of a mammalian RapR7 protein.

10. The fragment of claim 9, wherein said mammalian RapR7 protein is a human RapR7 protein, and wherein said fragment comprises amino acids 163-190 of said RapR7 protein.

11. The fragment of claim 6, wherein said fragment comprises a second peroximal domain of a mammalian RapR7 protein.

12. The fragment of claim 11, wherein said mammalian RapR7 protein is a human RapR7 protein, and wherein said fragment comprises amino acids 514-522 of said RapR7 protein.

13. A molecule comprising the fragment of any one of claims 6-12.

14. A protein comprising an amino acid sequence that has at least 60% identity to a domain of a mammaliann RapR7 protein, in which the percentage identity is determined over an amino acid sequence of identical size to the domain.

15. A protein comprising an amino acid sequence that has at least 90% identity to a domain of a mammalian RapR7 protein, in which the percentage identity is determined over an amino acid sequence of identical size to the domain.

16. A polypeptide comprising a fragment of a mammalian RapR7 protein consisting of at least 6 amino acids fused via a covalent bond to an amino acid sequence of a second peptide, wherein said second peptide is not comprised in a mammalian RapR7 protein.

17. The polypeptide of claim 16, wherein the fragment of the mammalian RapR7 protein is a fragment capable of binding to an anti-RapR7 protein antibody.

18. The polypeptide of claim 17, wherein the fragment capable of binding to an anti-RapR7 protein antibody further lacks one or more domains of the RapR7 protein.

19. An antibody which is capable of binding to a mammalian RapR7 protein.

20. The antibody of claim 19 which is a monoclonal antibody.

21. A molecule comprising a fragment of the antibody of claim 19, wherein said fragment is capable of binding to a RapR7 protein.

22. An isolated nucleic acid comprising a nucleotide sequence encoding a mammalian RapR7 protein as set forth in SEQ ID NO:2 or 5.

23. The nucleic acid of claim 22 which is DNA.

24. An isolated nucleic acid comprising a nucleotide sequence complementary to the nucleotide sequence of claim 22.

25. An isolated nucleic acid hybridizable to the nucleic acid of claim 22.

26. An isolated nucleic acid comprising a fragment of a mammalian RapR7 gene consisting of at least 8 nucleotides.

27. An isolated nucleic acid comprising a fragment of a mammalian RapR7 gene comprising any one of exons 1-11 of a mammalian RapR7 gene.

28. An isolated nucleic acid comprising a fragment of a mammalian RapR7 gene comprising an intron, or a fragment thereof, of a mammalian RapR7 gene.

29. An isolated nucleic acid comprising a nucleotide sequence encoding a fragment of a mammalian RapR7 protein that displays one or more functional activities of the mammalian RapR7 protein.

30. An isolated nucleic acid comprising a nucleotide sequence encoding any one of the fragments of claims 6-12.

31. A recombinant cell containing the nucleic acid of claim 29.

32. A method of producing a mammalian RapR7 protein comprising:
(a) growing a recombinant cell containing the nucleic acid of claim 29 such that the encoded fragment of said mammalian RapR7 protein is expressed by the cell; and
(b) recovering said expressed fragment of said mammalian RapR7 protein.

33. The product of the process of claim 32.

34. A pharmaceutical composition comprising a therapeutically effective amount of a mammalian RapR7 protein and a pharmaceutically acceptable carrier.

35. A pharmaceutical composition comprising a therapeutically effective amount of an antibody capable of binding to a mammalian RapR7 protein and a pharmaceutically acceptable carrier.

36. A method for generating a genetically modified cell having altered sensitivity to rapamycin, said method comprising introducing into the genome of a cell of a selected cell type of an organism a knockout DNA construct, said knockout DNA construct comprising (i) a regulated promoter and (ii) a selection marker coding sequence under the control of said regulated promoter, wherein said regulated promoter, when activated, initiates RNA transcription to produce an RNA; wherein, when said regulated promoter is activated, said genetically modified cell is rapamycin resistant if cells of said selected cell type is rapamycin sensitive or is rapamycin sensitive if cells of said selected cell type is rapamycin resistant.

37. The method of claim 36, wherein said knockout DNA construct further comprises a rapid cloning element comprising a replication origin sequence comprising sequences for initiation of replication and segregation and a bacterial selection marker.

38. The method of claim 37, wherein said replication origin sequence is an Ori and said bacterial selection marker is a chloramphenicol resistance gene.

39. The method of claim 36, wherein said method further comprising activating said regulated promoter and identifying said genetically modified cell by a method comprising identifying a change in rapamycin resistance in said genetically modified cell.

40. The method of claim 37, further comprising cloning a fragment of genomic sequence by a method comprising: (a) obtaining a nucleotide sequence comprising said rapid cloning element and said fragment of genomic sequence; (b) circularizing said nucleotide sequence to generate a circular plasmid; and (c) transforming a suitable host cell using said circular plasmid.

41. The method of claim 39, further comprising determining the sequence of said fragment of genomic sequence by a method comprising sequencing said circular plasmid.

42. The method claim 40, further comprising determining the location of said fragment of genomic sequence in said genome of said cell by a method comprising comparing said sequences with the genomic sequence of said selected cell type.

43. The method of claim 40, wherein said method further comprising, prior to said step of introducing said knockout DNA construct, introducing into the genome of cells of said selected cell type a DNA construct encoding a transactivator, said DNA construct comprising (i) a promoter and (ii) a nucleotide sequence encoding a transactivator, said nucleotide sequence being under the control of said promoter, wherein said regulated promoter is activated by said transactivator, and wherein said genetically modified cell is generated by introducing said knockout DNA construct into a cell comprising said DNA construct encoding said transactivator.

44. The method of claim 43, wherein said regulated promoter is a tetracycline regulated promoter, and wherein said transactivator activates said regulated promoter in the absence of tetracycline.

45. The method of claim 44, wherein said knockout DNA construct further comprises a rapid cloning element comprising a replication origin sequence comprising sequences for initiation of replication and segregation and a bacterial selection marker.

46. The method of claim 45, wherein said replication origin sequence is an Ori and said bacterial selection marker is a chloramphenicol resistance gene.

47. The method of claim 46, wherein said method further comprising identifying said genetically modified cell by a method comprising identifying a change in rapamycin resistance in said genetically modified cell.

48. The method of claim 46, further comprising cloning a fragment of genomic sequence by a method comprising: (a) obtaining a nucleotide sequence comprising said rapid cloning element and said fragment of genomic sequence; (b) circularizing said nucleotide sequence to generate a circular plasmid; and (c) transforming a suitable host cell using said circular plasmid.

49. The method of claim 48, further comprising determining the sequence of said fragment of genomic sequence by a method comprising sequencing said circular plasmid.

50. The method of claim 49, further comprising determining the location of said fragment of genomic sequence in said genome of said cell by a method comprising comparing said sequences with the genomic sequence of said selected cell type.

51. The method of any one of claims 36-50, wherein said selected cell type is a rapamycin sensitive cell type.

52. The method of claim 51, wherein said organism is a human.

53. The method of claim 51, wherein said organism is a mouse.

54. The method of claim 53, wherein said selected cell type is the murine neuroblastoma N2a cell line.

55. The method of claim 51, wherein said knockout DNA construct is integrated at a location in a RapR7 gene.

56. The method of claim 55, wherein said knockout DNA construct is integrated before the coding region of said RapR7 gene such that the expression of said RapR7 gene is activated or enhanced.

57. The method of any one of claims 36-50, wherein said selected cell type is a rapamycin resistant cell type.

58. The method of claim 57, wherein said organism is a human.

59. The method of claim 57, wherein said organism is a mouse.

60. A method for treating a mammal having a cancer, said cancer being caused by defective regulation of a RapR7 gene and/or defective activity of a protein encoded by said RapR7 gene, said method comprising administering to said mammal a therapeutically sufficient amount of an agent, said agent regulating the expression of said RapR7 gene and/or activity of said protein encoded by said RapR7 gene.

61. The method of claim 60, wherein said cancer is caused by an increase of expression of said RapR7 gene, and wherein said agent reduces the expression of said RapR7 gene in cells of said cancer.

62. The method of claim 60, wherein said cancer is caused by a mutation in said RapR7 gene, and wherein said agent causes the expression of a normal version of said RapR7 gene in cells of said cancer.

63. A method for treating a mammal having a cancer, comprising administering to said mammal a therapeutically sufficient amount of an agent, said agent regulating the expression of a RapR7 gene and/or activity of a protein encoded by said RapR7 gene such that rapamycin resistance is regulated, wherein said mammal is subject to a therapy comprising administrating to said mammal a therapeutically sufficient amount of rapamycin or an analog or derivative of rapamycin.

64. A method for treating a mammal having a cancer, comprising administering to said mammal i) a therapeutically sufficient amount of an agent, said agent regulating the expression of a RapR7 gene and/or activity of a protein encoded by said RapR7 gene such that rapamycin resistance is regulated, and ii) a therapeutically sufficient amount of rapamycin or an analog or derivative of rapamycin.

65. The method of claim 63 or 64, wherein said agent reduces the expression of said RapR7 gene in cells of said cancer.

66. The method of claim 63 or 64, wherein said agent causes the expression of a normal version of said RapR7 gene in cells of said cancer.

67. The method of claim 63 or 64, wherein said agent comprises a RapR7 protein or a therapeutically equivalent fragment thereof.

68. A method for diagnosing a cancer or a predisposition to said cancer in a mammal, said cancer being a result of defective regulation of a RapR7 gene, said method comprising determining an expression level of said RapR7 gene in cells of said mammal, wherein said expression level below a predetermined threshold level indicates that said mammal has or is predisposed of said cancer.

69. The method of claim 68, wherein said expression level of said RapR7 gene is determined by a method comprising measuring the expression level of said RapR7 gene using one or more polynucleotide probes, each of said one or more polynucleotide probes comprising a nucleotide sequence in said RapR7 gene.

70. The method of claim 69, wherein said one or more polynucleotide probes comprise at least one polynucleotide probe comprising a nucleotide sequence within one of exons 1-11 of said RapR7 gene.

71. The method of claim 69, wherein said one or more polynucleotide probes comprise at least one polynucleotide probe comprising a nucleotide sequence of said RapR7 gene which is not comprised in a nucleotide sequence that encodes a PHD domain, a coiled-coil domain, a second peroximal domain, a nuclear localization domain or a low complexity domain of the encoded RapR7 protein.

72. The method of claim 69, wherein said one or more polynucleotide probes comprise at least one polynucleotide probe comprising a nucleotide sequence within an intron of said RapR7 gene.

73. The method of claim 69, wherein said one or more polynucleotide probes comprise at least one polynucleotide probe comprising a nucleotide sequence comprised in the nucleotide sequence encoding a PHD domain, a coiled-coil domain, a second peroximal domain, a nuclear localization domain or a low complexity domain in the encoded RapR7 protein.

74. The method of any one of claims 68-73, wherein said one or more polynucleotide probes are polynucleotide probes on a microarray.

75. A method for diagnosing a cancer or a predisposition to said cancer in a mammal, said cancer being a result of defective regulation of a RapR7 gene, said method comprising determining a level of abundance of a protein encoded by said RapR7 gene in cells of said mammal, wherein said level of abundance of said protein above a predetermined threshold level indicates that said mammal has or is predisposed of said cancer.

76. A method for diagnosing a cancer or a predisposition to said cancer in a mammal, said cancer being a result of defective regulation of a RapR7 gene, said method comprising determining a level of activity of a protein encoded by said RapR7 gene in cells of said mammal, wherein said activity level above a predetermined threshold level indicates that said mammal has or is predisposed of said cancer.

77. The method of claim 75 or 76, wherein said mammal is a human.

78. The method of claim 77, wherein said protein is a human RapR7 protein as depicted in SEQ ID NO:6 or 7.

79. The method of claim 75 or 76, wherein said mammal is a mouse.

80. The method of claim 77, wherein said protein is murine RapR7 protein as depicted in SEQ ID NO:3 or 4.

81. A method for evaluating rapamycin resistance in a cell, said method comprising determining an expression level of a RapR7 gene in said cell, wherein said expression level above a predetermined threshold level indicates that said cell is rapamycin resistant.

82. The method of claim 81, wherein said expression level of said RapR7 gene is determined by a method comprising measuring the expression level of said RapR7 gene sing one or more polynucleotide probes, each of said one or more polynucleotide probes comprising a nucleotide sequence in said RapR7 gene.

83. The method of claim 82, wherein said one or more polynucleotide probes comprise at least one polynucleotide probe comprising a nucleotide sequence within one of exons 1-11 of said RapR7 gene.

84. The method of claim 82, wherein said one or more polynucleotide probes comprise at least one polynucleotide probe comprising a nucleotide sequence of said RapR7 gene which is not comprised in a nucleotide sequence that encodes a PHD domain, a coiled-coil domain, a second peroximal domain, a nuclear localization domain or a low complexity domain of the encoded RapR7 protein.

85. The method of claim 83, wherein said one or more polynucleotide probes comprise at least one polynucleotide probe comprising a nucleotide sequence within an intron of said RapR7 gene.

86. The method of any one of claims 81-85, wherein said one or more polynucleotide probes are polynucleotide probes on a microarray.

87. The method of claim 86, wherein said one or more polynucleotide probes comprise at least one polynucleotide probe comprising a nucleotide sequence comprised in the nucleotide sequence encoding a PHD domain, a coiled-coil domain, a second peroximal domain, a nuclear localization domain or a low complexity domain in a RapR7 protein.

88. A method for evaluating rapamycin resistance in a cell, said method comprising determining a level of abundance of a protein encoded by a RapR7 gene in said cell, wherein said level of abundance of said protein above a predetermined threshold level indicates that said cell is rapamycin resistant.

89. A method for evaluating rapamycin resistance in a cell, said method comprising determining a level of activity of a protein encoded by a RapR7 gene in said cell, wherein said activity level above a predetermined threshold level indicates that said cell is rapamycin resistant.

90. The method of claim 88 or 89, wherein said cell is a human cell.

91. The method of claim 90, wherein said protein is a human RapR7 protein as depicted in SEQ ID NO:6 or 7.

92. The method of claims 88 or 89, wherein said cell is a murine cell.

93. The method of claim 92, wherein said protein is murine RapR7 protein as depicted in SEQ ID NO:3 or 4.

94. A method for regulating rapamycin resistance in a cell, comprising contacting said cell with a sufficient amount of an agent such that rapamycin resistance is regulated, said agent regulating the expression of a RapR7 gene and/or the activity of a protein encoded by said RapR7 gene.

95. A method for regulating rapamycin resistance in a mammal, comprising administering to said mammal a therapeutically sufficient amount of an agent such that rapamycin resistance is regulated, said agent regulating the expression of a RapR7 gene and/or the activity of a protein encoded by said RapR7 gene.

96. A method for regulating growth of a cell, comprising contacting said cell with i) a sufficient amount of an agent such that rapamycin resistance is regulated, said agent regulating the expression of a RapR7 gene and/or the activity of a protein encoded by said RapR7 gene; and ii) a sufficient amount of rapamycin or an analog or derivative of rapamycin.

97. The method of claims 94, 95 or 96, wherein said agent reduces the expression of said RapR7 gene in said cell.

98. The method of claims 94, 95 or 96, wherein said agent causes the expression of a normal version of said RapR7 gene in said cell.

99. The method of claims 94, 95 or 96, wherein said agent comprises a RapR7 protein or a therapeutically equivalent fragment thereof.

100. A method of identifying an agent that is capable of regulating rapamycin resistance, wherein said agent is capable of modulating the expression of a RapR7 gene and/or the activity of a protein encoded by said RapR7 gene, said method comprising comparing inhibitory effect of rapamycin on cells expressing said RapR7 gene in the presence of said agent with inhibitory effect of rapamycin on cells expressing said RapR7 gene in the absence of said agent, wherein a difference in said inhibitory effect of rapamycin identifies said agent as capable of regulating rapamycin resistance.

101. A method of identifying an agent that is capable of regulating rapamycin resistance, wherein said agent is capable of modulating the expression of a RapR7 gene and/or activity of a protein encoded by said RapR7 gene, said method comprising:
(a) contacting a first cell expressing said RapR7 gene with rapamycin in the presence of said agent and measuring a first growth inhibitory effect;
(b) contacting a second cell expressing said RapR7 gene with rapamycin in the absence of said agent and measuring a second growth inhibitory effect; and
(c) comparing said first and second inhibitory effects measured in said step (a) and (b),
wherein a difference between said first and second inhibitory effects identifies said agent as capable of regulating rapamycin resistance.

102. The method of claims 100 or 101, wherein said agent comprises a molecule which reduces expression of said RapR7 gene.

103. The method of claims 100 or 101, wherein said agent causes the expression of a normal version of said RapR7 gene in a cell.

104. A method of producing an antibody that binds specifically to a RapR7 protein, comprising raising said antibody against said RapR7 protein or a polypeptide comprising an fragment of said RapR7 protein.

105. The method of claim 104, wherein said RapR7 protein is a human RapR7 protein.

106. The method of claim 104, wherein said RapR7 protein is a murine RapR7 protein.

107. The method of claim 104, wherein said fragment of said RapR7 protein comprises a PHD domain, a coiled-coil domain, a second peroximal domain, a nuclear localization domain or a low complexity domain of said RapR7 protein.

108. The method of claim 107, wherein said fragment comprises amino acids 217-263 or 326-381 of said RapR7 protein, or a fragment thereof.

109. An antibody that binds specifically to a RapR7 protein or a fragment of said RapR7 protein such that binding of said antibody to said RapR7 protein regulates rapamycin resistance.

110. The antibody of claim 109, wherein said RapR7 protein is a human RapR7 protein.

111. The antibody of claim 109, wherein said RapR7 protein is a murine RapR7 protein.

112. The antibody of claim 109, wherein said fragment of said RapR7 protein comprises a PHD domain, a coiled-coil domain, a second peroximal domain, a nuclear localization domain or a low complexity domain of said RapR7 protein.

113. The method of claim 109, wherein said fragment comprises amino acids 163-190 or 514-522 of said RapR7 protein, or a fragment thereof.

114. An agent that regulates the expression of a RapR7 gene such that rapamycin resistance is regulated.

115. The agent of claim 114, wherein said agent comprises a molecule which regulates expression of said RapR7 gene.

116. The agent of claim 115, wherein said molecule reduces expression of said RapR7 gene.

117. The agent of claim 114, wherein said agent blocks or reduces the binding of a regulator to said RapR7 gene.

118. The agent of claim 117, wherein said regulator is an activator of said RapR7 gene.

119. The agent of claim 114, wherein said agent causes the expression of a normal version of said RapR7 gene in a cell.

120. A cell comprising a knockout DNA construct at a RapR7 locus, said knockout DNA construct comprising (i) a regulated promoter and (ii) a selection marker coding sequence under the control of said regulated promoter, wherein said regulated promoter, when activated, initiates RNA transcription of said RapR7 gene.

121. The cell of claim 120, further comprising a DNA construct encoding a transactivator, said DNA construct comprising (i) a promoter and (ii) a nucleotide sequence encoding said transactivator, said nucleotide sequence being under the control of said promoter, wherein said transactivator activates said regulated promoter.

122. The cell of claim 120, wherein said knockout DNA construct further comprises a rapid cloning element comprising a replication origin sequence comprising sequences for initiation of replication and segregation and a bacterial selection marker.

123. The cell of claim 122, wherein said replication origin sequence is an Ori and said bacterial selection marker is a chloramphenicol resistance gene.

124. The cell of claim 123, wherein said regulated promoter is a tetracycline regulated promoter, and wherein said transactivator activates said regulated promoter in the absence of tetracycline.

125. The cell of any one of claims 120-124, wherein said cell is a rapamycin sensitive cell.

126. The cell of claim 125, wherein said cell is a human cell.

127. The cell of claim 125, wherein said cell is a murine cell.

128. The cell of claim 127, wherein said cell is a murine neuroblastoma N2a cell.

129. The cell of claim 127, wherein said integration site is in the intron between exon 1 and exon 2 of said RapR7 locus.

130. The cell of any one of claims 120-124, wherein said cell is a rapamycin resistant cell.

131. A microarray for diagnosing rapamycin resistance, said microarray comprising one or more polynucleotide probes, wherein each said polynucleotide probe comprises a nucleotide sequence in a RapR7 gene.

132. The microarray of claim 131, wherein said one or more polynucleotide probes comprise at least one polynucleotide probe comprising a nucleotide sequence within one of exons 1-11 of said RapR7 gene.

133. The microarray of claim 131, wherein said one or more polynucleotide probes comprise at least one polynucleotide probe comprising a nucleotide sequence within an intron of said RapR7 gene.

134. A kit for diagnosis of rapamycin resistance, comprising in one or more containers one or more polynucleotide probes, wherein each said polynucleotide probe comprises a nucleotide sequence in a RapR7 gene.

135. A kit for screening for agents which regulate rapamycin resistance and/or tumorgenesis, comprising in one or more containers (i) the cell of claim 120; (ii) tetracycline or a derivative or analog thereof; and (iii) rapamycin or a derivative or analog thereof.

136. The protein of claim 1 which comprises the amino acid sequence substantially as set forth in SEQ ID NO:3 or 6.

137. The polypeptide of claim 18, wherein said one or more domains of the RapR7 protein are selected from the group consisting of a PHD domain, a coiled-coil domain, a second peroximal domain, a nuclear localization domain and a low complexity domain of said RapR7 protein.


SEQUENCE LISTING (23 sequences)

SEQ ID NO: 1 - DNA, 1886 bp, Murine
  misc_feature (3)..(4): n is a, c, g, or t
tcnnattcccgagaataaatttctgtgactaactcttccttttgttggttcttcatggcatatgccttatgaaggtaacatacccaagctgcctctgcctcccgcagtgaacccctaccctgccctttggcaggttctcttactgaccatccccacctgccccacacatcctcccctatgcaccccaactntgagcccctcctgctcagtaagtctgtagacttggtgggtatattggnctcattgagactgcaggcccttggagggcaggctctgacctgcagtaagatgtgtgagtgatactcagcacacantaggtggataaatacccccacagtaggtgggtagtgagccctgtgagtccactgtaagncaccatctacatgggcanagcctgctttaagcgtgggttagggacacaacagtctcttcagcagggcttctggcaccatctacacaagtccatcctcagctcttccactcccgggttccctcctggacctgtgtgactctgaggaacttggggaattcctaacctcccctttcaactgagcccttggctcttggagttagccacaacctaactactcaggtccctccaacaaggggactgtgtctgtggctggatgactcatgcacactgctccatcccgcaatcttgggcgggacttgggctggggaggatgccagccagctcaggctaggagcttgcatcctgttgccccaacccagccctaccagaacagagtgtactcagagctccaggacaaaaatctggaaacagagagccggctctcatttggaccgagatctgagtgatgaaaagagcaggcagaggaaacagcaagttcaaagttcctgaggtgggaatgcgcttgacacaacggagacctgagaagaacacagcaaaggccgtgttacatttgtctgngactccagcccccaaggatctggtcaggacagacatngcgaggactcacctggataatccagagccatggcccatnacangnntncttcttttttttttttccttcttttttctttttctctttttttgnnnnnggccccaagacaggctttctttgngtagccccggctgttttggaactnactntgtagaccaaactggcctgngaactcacagagatcctcctgnctttgnctnccgagtacaagggttaaaagcctgagccantaccactggccaggctaactaaggttcttaactttttaagnattatttttctttcttatgtatgtgtatatgggggaggggatgcacaagggcatggggggggggtccctgcagaagtcagaagaggtgccagatccctgggagctggaattaaagtcagtcatgaaacatccaagatggacactgggnaactgaacttgggtcctctgcgagaggagtaatggtcttaactgctgagccatctctaggcccaatgtctggttttgttttgttttgttttgttttgttttgttttgttttgttttgtatttgggggtttttgtttgtctgtttggttggtttgtttggtttttcttgagacagggtttctctgtatagccctggctgtcctggaactcactctatagactaggctggcctcgaactcagaaatcctcctgcctctgcctcccaagtgctgagattaaaggcccgtgccaccactgcccgacgccaatgtctgtattttattcatctctgcagaatctcttttgtctcctaacggaacatcatcccagattctgggaagtacactgaagacaatggggtgggtgttgtttctctcctatgccctttacatnctccctacctatttcagatgtaaccatgatctaccagctcatcacaggccacagcttaaacctccctc

SEQ ID NO: 2 - DNA, 2856 bp, Murine
atgagtgagtctatactcacaggcactgagaaagccagactcaacggctacctcctccaagatgtaaccatgatctaccagctcatcacaggccacagcttaaacctccctcccctttgtcacatctccaccatcaaccacacccttccatctttctcttcatctgacacatatcttccaacccttcagtcatctaataagcagactttaaaagccacgggtcctggatatccaatggaaaatgaccaaaggaagaacacttgctccttagtccgacaagaaggtttcaaaggagtcaccttgcatgctgaagcacttcccacagaaggagcacccccccccccacctcatctgcaggattccgagatggaagagaagaggcgaaaatattccatcagcagcgacaactctgataccactgacggtcacgtgacatccacatcagcatcaagatgttccaaactgcccagcagcaccaagtcgggctggccccggcagaacgagaagaagccctcagaggttttccggacagacttgatcacagccatgaagatcccagattcataccagctcagcccggatgactactacatcctggcggacccgtggcgacaagaatgggagaaaggggtgcaggtacctgctggagcggaggccattccagagcctgtggtgaggctcctcccaccactgaaaggcccccccacgcagatgtccccagatagccccacacttggtgagggtgcccatcctgactggccaggaggcagccgctacgacctggatgagatcgatgcgtactggttggaacttctcaactcggagctcaaggagatggagaagcccgagctggatgagctaacgttagagcgtgttctagaggagctagagacattgtgccaccagaatatggcacaggccattgagacacaggaggggctgggcatcgagtacgacgaggacgttgtctgcgacgtgtgccgttcccctgaaggcgaggatggcaacgagatggtcttctgtgacaaatgcaatgtctgtgtgcaccaggcatgctacgggatcctcaaggtgcctacgggcagctggctgtgccggacctgtgccctgggagtccagcctaagtgcctgctctgccccaagcgaggaggagccctgaagcccactagaagtgggaccaagtgggtacacgtcagctgtgccctgtggattcctgaggtcagcattggctgtccagagaagatggagcccattaccaagatctcgcatattccggccagccgctgggccctgtcctgcagcctctgcaaggagtgcacaggtacctgcatccagtgttccatgccttcctgcatcacagcattccacgttacgtgcgcctttgaccgaggcctggaaatgcggactatattagctgacaatgacgaggtcaagttcaagtcactttgccaggagcacagtgacgggggccctcggagtgagcctacttctgagcctgtggagcccagccaggccgttgaggatctggaaaaggtgaccttacgcaagcagcggctgcagcagctggaagaaaacttctatgagctagtggagccagctgaggtggctgaacggctagacctggctgaggcactggtggacttcatctaccagtactggaagttgaagcggagagctaatgccaaccagccgctgttgacgcccaagactgacgaggtggacaacctggcccaacaggaacaggatgtcctctatcgacgcctgaagcttttcacccacctgcggcaggacctggagagggtaaggaacctgtgctacatggtgacaagacgggagagaacgaaacacaccatctgtaaacttcaggagcagatattccatctacagatgaaacttattgagcaagacctttgcagagagccttctgggaggaggtcaaagggcaagaagaatgattcaaaaaggaaaggccgagagggtcccaagggcagcagccctgagaagaaagagaaagtgaaggctgggcccgagtctgtgctggggcagctgggtctatccacctcgttccccatcgacggcactttcttcaacagctggttggcacagtcggttcagatcacagcagaggacatggccatgagcgagtggtctttgaacagtgggcaccgggaggatcctgctccaggtctgctgtcagaggaattgctacaagatgaggagacgctgctcagcttcatgagggacccctcgctacgacctggtgaccctgccagaaaggcccgaggccgcactcgcctgcctgccaagaagaaaccatccccgctgcaggatgggcccagtgcacggaccactccagacaagcaacccaagaaggcctgggcccaggatggcaaggggacgcaaggaccacccatgaggaagccaccacggaggacgtcttctcatttgccgtccagccctgcagctggggactgtccagtcccagcaacactggaaagccctccaccactggcctccgagatactagacaagacagcccccatggcttccgacttaaatgtccaagtgcctggccctacagtgagccccaaacccttgggcaggctccggccaccccgagagatgaaggtcagtcggaaatctccgggtgctagatccgatgctgggacaggactaccgtctgctgtggccgagaggccaaaggtcagcctgcattttgacaccgaggctgacggctacttctctgatgaggagatgagcgattctgaggtagaggcagaggacagtggggtacaacgagcttccagggaggcaggggcagaggaggtggttcgcatgggggtgctggcctcctaa

SEQ ID NO: 3 - protein, 951 aa, Murine
MSESILTGTEKARLNGYLLQDVTMIYQLITGHSLNLPPLCHISTINHTLPSFSSSDTYLPTLQSSNKQTLKATGPGYPMENDQRKNTCSLVRQEGFKGVTLHAEALPTEGAPPPPPHLQDSEMEEKRRKYSISSDNSDTTDGHVTSTSASRCSKLPSSTKSGWPRQNEKKPSEVFRTDLITAMKIPDSYQLSPDDYYILADPWRQEWEKGVQVPAGAEAIPEPVVRLLPPLKGPPTQMSPDSPTLGEGAHPDWPGGSRYDLDEIDAYWLELLNSELKEMEKPELDELTLERVLEELETLCHQNMAQAIETQEGLGIEYDEDVVCDVCRSPEGEDGNEMVFCDKCNVCVHQACYGILKVPTGSWLCRTCALGVQPKCLLCPKRGGALKPTRSGTKWVHVSCALWIPEVSIGCPEKMEPITKISHIPASRWALSCSLCKECTGTCIQCSMPSCITAFHVTCAFDRGLEMRTILADNDEVKFKSLCQEHSDGGPRSEPTSEPVEPSQAVEDLEKVTLRKQRLQQLEENFYELVEPAEVAERLDLAEALVDFIYQYWKLKRRANANQPLLTPKTDEVDNLAQQEQDVLYRRLKLFTHLRQDLERVRNLCYMVTRRERTKHTICKLQEQIFHLQMKLIEQDLCREPSGRRSKGKKNDSKRKGREGPKGSSPEKKEKVKAGPESVLGQLGLSTSFPIDGTFFNSWLAQSVQITAEDMAMSEWSLNSGHREDPAPGLLSEELLQDEETLLSFMRDPSLRPGDPARKARGRTRLPAKKKPSPLQDGPSARTTPDKQPKKAWAQDGKGTQGPPMRKPPRRTSSHLPSSPAAGDCPVPATLESPPPLASEILDKTAPMASDLNVQVPGPTVSPKPLGRLRPPREMKVSRKSPGARSDAGTGLPSAVAERPKVSLHFDTEADGYFSDEEMSDSEVEAEDSGVQRASREAGAEEVVRMGVLAS

SEQ ID NO: 4 - protein, 829 aa, Murine
MEEKRRKYSISSDNSDTTDGHVTSTSASRCSKLPSSTKSGWPRQNEKKPSEVFRTDLITAMKIPDSYQLSPDDYYILADPWRQEWEKGVQVPAGAEAIPEPVVRLLPPLKGPPTQMSPDSPTLGEGAHPDWPGGSRYDLDEIDAYWLELLNSELKEMEKPELDELTLERVLEELETLCHQNMAQAIETQEGLGIEYDEDVVCDVCRSPEGEDGNEMVFCDKCNVCVHQACYGILKVPTGSWLCRTCALGVQPKCLLCPKRGGALKPTRSGTKWVHVSCALWIPEVSIGCPEKMEPITKISHIPASRWALSCSLCKECTGTCIQCSMPSCITAFHVTCAFDRGLEMRTILADNDEVKFKSLCQEHSDGGPRSEPTSEPVEPSQAVEDLEKVTLRKQRLQQLEENFYELVEPAEVAERLDLAEALVDFIYQYWKLKRRANANQPLLTPKTDEVDNLAQQEQDVLYRRLKLFTHLRQDLERVRNLCYMVTRRERTKHTICKLQEQIFHLQMKLIEQDLCREPSGRRSKGKKNDSKRKGREGPKGSSPEKKEKVKAGPESVLGQLGLSTSFPIDGTFFNSWLAQSVQITAEDMAMSEWSLNSGHREDPAPGLLSEELLQDEETLLSFMRDPSLRPGDPARKARGRTRLPAKKKPSPLQDGPSARTTPDKQPKKAWAQDGKGTQGPPMRKPPRRTSSHLPSSPAAGDCPVPATLESPPPLASEILDKTAPMASDLNVQVPGPTVSPKPLGRLRPPREMKVSRKSPGARSDAGTGLPSAVAERPKVSLHFDTEADGYFSDEEMSDSEVEAEDSGVQRASREAGAEEVVRMGVLAS

SEQ ID NO: 5 - DNA, 2750 bp, Homo sapiens
gttttaaaaagaaacagaaacatacacagggggttggtgaatggtgccgaccgcggccatcgcagttggaggctattttttgggggggatggaagagaagaggcgaaaatactccatcagcagtgacaactctgacaccactgacagtcatgcgacatctacatccgcatcaagatgctccaaactgcccagcagcaccaagtcgggctggccccgacagaacgaaaagaagccctccgaggttttccggacagacttgatcacagccatgaagatcccggactcataccagctcagcccggatgactactacatcctggcagacccatggcgacaggaatgggagaaaggtgtgcaggtgcctgccggggcagaggccatcccagagcccgtggtgaggatcctcccaccactggaaggcccccctgcccaggcatccccgagcagcaccatgcttggtgagggctcccagcctgattggccagggggcagccgctatgacttggacgagattgatgcctactggctggagctcatcaactcggagcttaaggagatggagaggccggagctggacgagctgacattagagcgtgtgctggaggagctggagaccctgtgccaccagaatatggccagggccattgagacgcaggaggggctgggcatcgagtacgacgaggatgttgtctgcgacgtgtgtcgctctcctgagggcgaggatggcaacgagatggtcttctgtgacaagtgcaacgtctgtgtgcatcaggcatgctacgggatcctcaaggtgcccacgggcagctggctgtgccggacgtgtgccctgggtgtccagccaaagtgcctgctctgccccaagcgaggaggagccttgaagcccactagaagtgggaccaagtgggtgcatgtcagctgtgccctatggattcctgaggtcagcatcggctgcccagagaagatggagcccatcaccaagatctcgcatatcccagccagccgctgggctctgtcctgcagcctctgcaaggaatgcacaggcacctgcatccagtgttccatgccttcctgcgtcacagcgttccatgtcacatgcgcctttgaccacggcctggaaatgcggactatattagcagacaacgatgaggtcaagttcaagtcattctgccaggagcacagtgacgggggcccacgtaatgagcccacatctgagcccacggaacccagccaggctggcgaggacctggaaaaggtgaccctgcgcaagcagcggctgcagcagctagaggaggacttctacgagctggtggagccggctgaggtggctgagcggctggacctggctgaggcactggtcgacttcatctaccagtactggaagctgaagaggaaagccaatgccaaccagccgctgctgacccccaagaccgacgaggtggacaacctggcccagcaggagcaggacgtcctctaccgccgcctgaagctcttcacccatctgcggcaggacctagagagggttagaaatctgtgctacatggtgacaaggcgcgagagaacgaaacacgccatctgcaaactccaggagcagatattccacctgcagatgaaacttattgaacaggatctgtgtcgagagcggtctgggaggagagcaaagggcaagaagagtgactcgaagaggaagggctgcgagggctccaagggcagcactgagaagaaagagaaagtgaaggcggggcctgactcagtcctggggcagctggcaggcctgtccacctcattccccatcgatggcaccttcttcaacagctggctggcacagtcggtgcagatcacagcagagaacatggccatgagcgagtggccactgaacaatgggcaccgcgaggaccctgctccagggctgctgtcagaggaactgctgcaggacgaggagacactgctcagcttcatgcgggacccctcgctgcgacctggtgaccctgctaggaaggcccgaggccgcacccgcctgcctgccaagaagaaaccaccaccaccaccaccgcaggacgggcctggttcacggacgactccagacaaagcccccaagaagacctggggccaggatgcaggcagtggcaaggggggtcaagggccacctaccaggaagccaccacgtcggacatcttctcacttgccgtccagccctgcagccggggactgtcccatcctagccacccctgaaagccccccgccactggcccctgagaccccggacgaggcagcctcagtagctgctgactcagatgtccaagtgcctggccctgcagcaagccctaagcctttgggccggctccggccaccccgcgagagcaaggtaacccggagattgccgggtgccaggcctgatgctgggatgggaccaccttcagctgtggctgagaggcccaaggtcagcctgcattttgacactgagactgatggctacttctctgatggggagatgagcgactcagatgtagaggccgaggacggtggggtgcagcggggtccccgggaggcaggggcagaggaggtggtccgcatgggcgtactggcctcctaactcacccccttccctgtcccaggccctgccctggtccccccacaaggcctcagcccagtcacaactgccatttccagtctctgctgagtgtcccagaccctcgaggctgccactccgtcgtggttttatttttaatatagagagagttttgaattct

SEQ ID NO: 6 - protein, 850 aa, Homo sapiens
MVPTAAIAVGGYFLGGMEEKRRKYSISSDNSDTTDSHATSTSASRCSKLPSSTKSGWPRQNEKKPSEVFRTDLITAMKIPDSYQLSPDDYYILADPWRQEWEKGVQVPAGAEAIPEPVVRILPPLEGPPAQASPSSTMLGEGSQPDWPGGSRYDLDEIDAYWLELINSELKEMERPELDELTLERVLEELETLCHQNMARAIETQEGLGIEYDEDVVCDVCRSPEGEDGNEMVFCDKCNVCVHQACYGILKVPTGSWLCRTCALGVQPKCLLCPKRGGALKPTRSGTKWVHVSCALWIPEVSIGCPEKMEPITKISHIPASRWALSCSLCKECTGTCIQCSMPSCVTAFHVTCAFDHGLEMRTILADNDEVKFKSFCQEHSDGGPRNEPTSEPTEPSQAGEDLEKVTLRKQRLQQLEEDFYELVEPAEVAERLDLAEALVDFIYQYWKLKRKANANQPLLTPKTDEVDNLAQQEQDVLYRRLKLFTHLRQDLERVRNLCYMVTRRERTKHAICKLQEQIFHLQMKLIEQDLCRERSGRRAKGKKSDSKRKGCEGSKGSTEKKEKVKAGPDSVLGQLAGLSTSFPIDGTFFNSWLAQSVQITAENMAMSEWPLNNGHREDPAPGLLSEELLQDEETLLSFMRDPSLRPGDPARKARGRTRLPAKKKPPPPPPQDGPGSRTTPDKAPKKTWGQDAGSGKGGQGPPTRKPPRRTSSHLPSSPAAGDCPILATPESPPPLAPETPDEAASVAADSDVQVPGPAASPKPLGRLRPPRESKVTRRLPGARPDAGMGPPSAVAERPKVSLHFDTETDGYFSDGEMSDSDVEAEDGGVQRGPREAGAEEVVRMGVLAS

SEQ ID NO: 7 - protein, 834 aa, Homo sapiens
MEEKRRKYSISSDNSDTTDSHATSTSASRCSKLPSSTKSGWPRQNEKKPSEVFRTDLITAMKIPDSYQLSPDDYYILADPWRQEWEKGVQVPAGAEAIPEPVVRILPPLEGPPAQASPSSTMLGEGSQPDWPGGSRYDLDEIDAYWLELINSELKEMERPELDELTLERVLEELETLCHQNMARAIETQEGLGIEYDEDVVCDVCRSPEGEDGNEMVFCDKCNVCVHQACYGILKVPTGSWLCRTCALGVQPKCLLCPKRGGALKPTRSGTKWVHVSCALWIPEVSIGCPEKMEPITKISHIPASRWALSCSLCKECTGTCIQCSMPSCVTAFHVTCAFDHGLEMRTILADNDEVKFKSFCQEHSDGGPRNEPTSEPTEPSQAGEDLEKVTLRKQRLQQLEEDFYELVEPAEVAERLDLAEALVDFIYQYWKLKRKANANQPLLTPKTDEVDNLAQQEQDVLYRRLKLFTHLRQDLERVRNLCYMVTRRERTKHAICKLQEQIFHLQMKLIEQDLCRERSGRRAKGKKSDSKRKGCEGSKGSTEKKEKVKAGPDSVLGQLAGLSTSFPIDGTFFNSWLAQSVQITAENMAMSEWPLNNGHREDPAPGLLSEELLQDEETLLSFMRDPSLRPGDPARKARGRTRLPAKKKPPPPPPQDGPGSRTTPDKAPKKTWGQDAGSGKGGQGPPTRKPPRRTSSHLPSSPAAGDCPILATPESPPPLAPETPDEAASVAADSDVQVPGPAASPKPLGRLRPPRESKVTRRLPGARPDAGMGPPSAVAERPKVSLHFDTETDGYFSDGEMSDSDVEAEDGGVQRGPREAGAEEVVRMGVLAS

SEQ ID NO: 8 - DNA, 329 bp, Homo sapiens
gggggttggtgaatggtgccgaccgcggccatcgcagttggaggctattttttggggggggtgagtagcgtccatggagttactttgcgcccactcctagcggcaccggcttaggtcctgcgggccgaccgtccccggcggggggcgtggggcctgggacgccgcgggcccggccgcctccctcgccgcgaccccggatggatgcgcgccccccgccctcccgcgccggccccaggagctcccggcttcgggagcatccttcccgcgccggtccctgcagcggcgcgtagccgagggcagcgcccgtcaggggggcaccgcggagcaag

SEQ ID NO: 9 - DNA, 58 bp, Homo sapiens
atggaagagaagaggcgaaaatactccatcagcagtgacaactctgacaccactgaca

SEQ ID NO: 10 - DNA, 95 bp, Homo sapiens
gtcatgcgacatctacatccgcatcaagatgctccaaactgcccagcagcaccaagtcgggctggccccgacagaacgaaaagaagccctccgag

SEQ ID NO: 11 - DNA, 158 bp, Homo sapiens
gttttccggacagacttgatcacagccatgaagatcccggactcataccagctcagcccggatgactactacatcctggcagacccatggcgacaggaatgggagaaaggtgtgcaggtgcctgccggggcagaggccatcccagagcccgtggtgag

SEQ ID NO: 12 - DNA, 161 bp, Homo sapiens
gatcctcccaccactggaaggcccccctgcccaggcatccccgagcagcaccatgcttggtgagggctcccagcctgattggccagggggcagccgctatgacttggacgagattgatgcctactggctggagctcatcaactcggagcttaaggagatgg

SEQ ID NO: 13 - DNA, 212 bp, Homo sapiens
agaggccggagctggacgagctgacattagagcgtgtgctggaggagctggagaccctgtgccaccagaatatggccagggccattgagacgcaggaggggctgggcatcgagtacgacgaggatgttgtctgcgacgtgtgtcgctctcctgagggcgaggatggcaacgagatggtcttctgtgacaagtgcaacgtctgtgtgcatcag

SEQ ID NO: 14 - DNA, 168 bp, Homo sapiens
gcatgctacgggatcctcaaggtgcccacgggcagctggctgtgccggacgtgtgccctgggtgtccagccaaagtgcctgctctgccccaagcgaggaggagccttgaagcccactagaagtgggaccaagtgggtgcatgtcagctgtgccctatggattcctgag

SEQ ID NO: 15 - DNA, 117 bp, Homo sapiens
gtcagcatcggctgcccagagaagatggagcccatcaccaagatctcgcatatcccagccagccgctgggctctgtcctgcagcctctgcaaggaatgcacaggcacctgcatccag

SEQ ID NO: 16 - DNA, 465 bp, Homo sapiens
tgttccatgccttcctgcgtcacagcgttccatgtcacatgcgcctttgaccacggcctggaaatgcggactatattagcagacaacgatgaggtcaagttcaagtcattctgccaggagcacagtgacgggggcccacgtaatgagcccacatctgagcccacggaacccagccaggctggcgaggacctggaaaaggtgaccctgcgcaagcagcggctgcagcagctagaggaggacttctacgagctggtggagccggctgaggtggctgagcggctggacctggctgaggcactggtcgacttcatctaccagtactggaagctgaagaggaaagccaatgccaaccagccgctgctgacccccaagaccgacgaggtggacaacctggcccagcaggagcaggacgtcctctaccgccgcctgaagctcttcacccatctgcggcaggacctagagagg

SEQ ID NO: 17 - DNA, 118 bp, Homo sapiens
gttagaaatctgtgctacatggtgacaaggcgcgagagaacgaaacacgccatctgcaaactccaggagcagatattccacctgcagatgaaacttattgaacaggatctgtgtcgag

SEQ ID NO: 18 - DNA, 818 bp, Homo sapiens
gcctgtccacctcattccccatcgatggcaccttcttcaacagctggctggcacagtcggtgcagatcacagcagagaacatggccatgagcgagtggccactgaacaatgggcaccgcgaggaccctgctccagggctgctgtcagaggaactgctgcaggacgaggagacactgctcagcttcatgcgggacccctcgctgcgacctggtgaccctgctaggaaggcccgaggccgcacccgcctgcctgccaagaagaaaccaccaccaccaccaccgcaggacgggcctggttcacggacgactccagacaaagcccccaagaagacctggggccaggatgcaggcagtggcaaggggggtcaagggccacctaccaggaagccaccacgtcggacatcttctcacttgccgtccagccctgcagccggggactgtcccatcctagccacccctgaaagccccccgccactggcccctgagaccccggacgaggcagcctcagtagctgctgactcagatgtccaagtgcctggccctgcagcaagccctaagcctttgggccggctccggccaccccgcgagagcaaggtaacccggagattgccgggtgccaggcctgatgctgggatgggaccaccttcagctgtggctgagaggcccaaggtcagcctgcattttgacactgagactgatggctacttctctgatggggagatgagcgactcagatgtagaggccgaggacggtggggtgcagcggggtccccgggaggcaggggcagaggaggtggtccgcatgggcgtactggcctcc

SEQ ID NO: 19 - DNA, 1440 bp, Homo sapiens
cggccctggggacagggcgggctaggggcgccccagagtccatggggagtccgggcccagggtgccagcaggcgtggtggtggggctgcgagggagggcacccttcccccacggggcccgcaacgctacctggactccccgccggagccaaacaactgggcggggggttgggggggcggcgacgggggtgtcgggagcggagatccgagtgaataagaaaaaagtggctactccccctccctcgctcctcctgcccgcccccaccccacccccaccccaacacattttttttttctaaagagatcacaaggaagtcttggtttaaaaagaaacagaaacatacacagggggttggtgaatggtgccgaccgcggccatcgcagttggaggctattttttggggggggtgagtagcgtccatggagttactttgcgcccactcctagcggcaccggcttaggtcctgcgggccgaccgtccccggcggggggcgtggggcctgggacgccgcgggcccggccgcctccctcgccgcgaccccggatggatgcgcgccccccgccctcccgcgccggccccaggagctcccggcttcgggagcatccttcccgcgccggtccctgcagcggcgcgtagccgagggcagcgcccgtcaggggggcaccgcggagcaaggtaagatccagcccccggcggatgggccctgcgcatctccacgacgttatttggcgtttttgcaacagatctgccagcgctcttcgctccctcgctctctcttgctcgctcgctccctctctctcctgctggctgcctgttctaggaagccagcgcggagaggggggggatgcacagcacaggggagagagattgcgcatgttggtcagtcgtgttttaaagagtacagtgcggggaggctgagaggggcgcatgcaacaacaacttttggaagggtgagcttggcgaccttctttattaatgactgcggcaaagcgcccccgggccggcgagggggcgcgggcgggcgggggcgcgccagggctgcaacttccccgcgggctccggccgggcgtaggggctgcggcgggagatgggtacggtggggaggtcgagcggcccgggcgggggctccgagaacctggagctatctgccctcctgtctccccgagtttcattttgttgatacgcagcacgtccgggcgccgaaccgggctgagccggtgcacatgacctcgcgctgggctcacgtgcagccggtccggtcccagacaccttccgggggccaccgcctccgccctgtcgccccctctcccggcccggtgcacgcgggcgctgcacgcgggggcagcatgctcggctcctggggttggaggctctgcacaaattagacagtttttttggaggggcgggggacaccctttccaggtgagtgtggagggtgcg

SEQ ID NO: 20 - DNA, 50 bp, Homo sapiens
cccgccggagccaaacaactgggcggggggttgggggggcggcgacgggg

SEQ ID NO: 21 - DNA, 50 bp, Homo sapiens
ccggcggggggcgtggggcctgggacgccgcgggcccggccgcctccctc

SEQ ID NO: 22 - DNA, 50 bp, Homo sapiens
accttctttattaatgactgcggcaaagcgcccccgggccggcgaggggg

SEQ ID NO: 23 - DNA, 50 bp, Homo sapiens
gcccccgggccggcgagggggcgcgggcgggcgggggcgcgccagggctg